(19) United States
(12) Patent Application Publication
(10) Pub. No.: US 2022/0118116 A1
(43) Pub. Date: Apr. 21, 2022

(54) ADHESIVE/ADSORPTION SWITCH ON NANOPARTICLES TO INCREASE TUMOR UPTAKE AND DELAY TUMOR CLEARANCE

(71) Applicant: The Johns Hopkins University, Baltimore, MD (US)

(72) Inventors: Stavroula Sofou, Baltimore, MD (US); Sarah Sally Stras, Baltimore, MD (US); Alaina Howe, Baltimore, MD (US); Aprameya Ganesh Prasad, Baltimore, MD (US); Dominick Salerno, Baltimore, MD (US)

(21) Appl. No.: 17/422,667

(22) PCT Filed: Jan. 29, 2020
(86) PCT No.: PCT/US20/15677

§ 371 Date: Jul. 13, 2021

Related U.S. Application Data
(60) Provisional application No. 62/798,137, filed on Jan. 29, 2019.

(51) Int. Cl. A61K 51/04; A61K 47/54; A61K 33/243; A61K 31/282; A61P 35/04

(52) U.S. Cl. CPC A61K 51/0408; A61K 47/544; A61P 35/04; A61K 31/282; A61K 33/243

(57) ABSTRACT

Lipid-based nanocarriers (liposomes) loaded with a chemotherapeutic agent and exhibiting interstitial drug release and intratumoral adhesion are disclosed. The lipid-based nanocarriers disclosed herein include an ‘adsorptive/adhesive switch’ on the nanocarriers surface with the aim to increase the tumor residence times of the drug delivery nanocarriers and to slow down their tumor clearing kinetics. The switch is designed to promote nanoparticle adsorption on cancer cells and/or the extracellular matrix (ECM) while keeping their internalization by cells to a minimum. This approach of drug delivery is key for interstitial release of highly-diffusive forms of therapeutics.

### FEDERALLY SPONSORED RESEARCH OR DEVELOPMENT

This invention was made with government support under grant number CBET 1510015 awarded by the National Science Foundation. The government has certain rights in the invention.

### BACKGROUND

The poor prognosis of many metastatic cancers, including triple negative breast cancer (TNBC), can be attributed largely to the lack of tumor selective therapeutic modalities that effectively deliver lethal doses of a therapeutic agent to the sites of metastatic disease. Tumor-selective drug delivery strategies that aim to improve uniformity in intratumoral drug distributions and to prolong exposure of cancer cells to delivered therapeutics may improve efficacy against TNBC and other metastatic cancers.

Cationic lipid vesicles are extensively used in cell transfection due to their ability to interact with the cell membrane and to deliver intracellularly their therapeutic cargo. In these interactions the critical role of the cationic charge in enabling close approximation of and lipid rearrangement/exchange between the apposing lipid membranes has been extensively studied. There are instances in drug delivery, however, where only the adhesion of lipid vesicles on cells and/or the extracellular matrix in tumors is desired and any form of internalization by cells must be avoided.

### SUMMARY

In some aspects, the presently disclosed subject matter provides a lipid-based nanocarrier of formula (I):

L-P—R₁ (I);

wherein: L is a phospholipid; P is a polyethylene glycol linker; and R₁ is a moiety having a tritratable cationic charge that becomes positively charged under a physiological pH of a tumor interstitium; wherein: R₁ is conjugated to a free end of the polyethylene glycol linker; the lipid-based nanocarrier adheres to a target cell or the extracellular matrix (ECM) thereof, and wherein internalization of the lipid-based nanocarrier by the target cell is minimized; and pharmaceutically acceptable salts thereof.

In certain embodiments, the compound of formula (I) has the following structure:

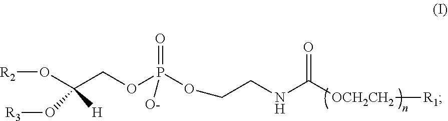

wherein: n is an integer from 1 to 1000; R₁ is a moiety having a tritratable cationic charge that becomes positively charged under a physiological pH of a tumor interstitium; R₂ and R₃ are each independently a fatty acid or fatty acid residue, wherein R₂ and R₃ can be the same or different; and pharmaceutically acceptable salts thereof.

In yet more particular aspects, R₁ comprises a moiety having an intrinsic pKa having a range from about 6.0 to about 6.9. In even yet more particular embodiments, R₁ is dimethyl ammonium propane.

In certain aspects, the polyethylene glycol linker is selected from the group consisting of PEG(100), PEG(200), PEG(300), PEG(400), PEG(600), PEG(800), PEG(1000), PEG(1500), PEG(2000), PEG(3000), PEG(3350), PEG(4000), PEG(6000), PEG(8000), PEG(10,000), and PEG(35,000). In more certain aspects, the polyethylene glycol linker comprises PEG(2000).

In certain aspects, R₂ and R₃ are each independently selected from the group consisting of: butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, nonatriacontanoic acid, tetracontanoic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid; stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, and herring acid.

In particular aspects, the compound of formula (I) has the following formula:

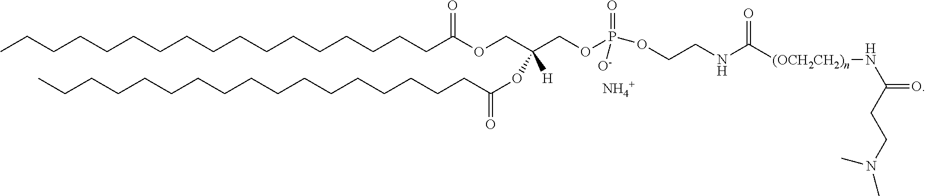

In other aspects, the lipid-based nanocarrier further comprises one or more therapeutic agents. In certain aspects, the one or more therapeutic agents comprises a chemotherapeutic agent. In other aspects, the one or more therapeutic agents comprises a radionuclide, such as an alpha-particle emitter (for example 225-Actinium) for internal radiotherapy. In more certain aspects, the chemotherapeutic agent comprises a platinum-based antineoplastic agent. In even yet more certain aspects, the platinum-based antineoplastic agent is selected from the group consisting of cisplatin, carboplatin, oxaliplatin, nedaplatin, triplatin tetranitrate, phenanthriplatin, picoplatin, and satraplatin.

In yet other aspects, the presently disclosed subject matter provides a pharmaceutical formulation comprising a lipid-based nanocarrier of Formula (I) a pharmaceutically acceptable carrier.

In some aspects, the presently disclosed subject matter provides a method for treating a disease, disorder, or condition, the method comprising administering a therapeutically effective amount of a lipid-based nanocarrier of Formula (I), or a formulation thereof, to a subject in need of treatment thereof.

In certain aspects, the disease, disorder, or condition comprises a cancer. In more certain aspects, the cancer comprises a metastatic cancer. In yet more certain aspects, the cancer is selected from the group consisting of testicular cancer, ovarian cancer, cervical cancer, breast cancer, bladder cancer, head and neck cancer, esophageal cancer, lung cancer, mesothelioma, brain tumors, and neuroblastoma. In even yet more certain aspects, cancer is breast cancer. In particular aspects, the breast cancer is triple negative breast cancer (TNBC).

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1 is the molecular structure of the presently disclosed ‘adhesion lipid’ DPPE-PEG(2000)-DAP (dimethyl ammonium propane). The DAP group, which is located at the free end of the PEG-chain, becomes positively charged with acidification of pH, for example in the slightly acidic pH of a tumor interstitium;

FIG. 2 shows the slightly acidic tumor interstitial pH (˜6.5) is utilized to trigger significant release of cisplatin from lipid nanoparticles. Liposomes with different combinations of the release (R) and the adhesion (A) properties are indicated as follows: R+A+(gray checkered bars), R+A− (gray solid bars), R-A+(white checkered bars), R-A− (white solid bars). Extent of cisplatin retention by lipid nanoparticles was measured at pH 7.4 and 6.5 following a 6 hour incubation in cell-conditioned media. Cell conditioned media were generated by incubation of media with cells overnight, followed by sterile filtration for use with the reported incubations of liposomes. Error bars correspond to standard deviations of repeated measurements (n=3 independent liposome preparations, 2 samples per preparation). ** indicates p-values<0.01;

FIG. 3A, FIG. 3B, and FIG. 3C show the location of the titratable cationic charge (DAP) relative to the lipid membrane affects the extent of association of lipid nanoparticles with cells. The effects on cell uptake of the extracellular pH and temperature (4° C. and 37° C.) also are shown. FIG. 3A: Liposomes containing DSPE-DAP (surface charge; i.e., DAP directly conjugated on lipid headgroups), composition numbered 1 on Table 1-2; FIG. 3B: Liposomes containing DSPE-PEG(2000)-DAP (‘adhesion lipid’; DAP conjugated on the free ends of the PEG-chains), composition numbered 2 on Table 1-2; FIG. 3C: Liposomes containing DSPE-PEG(2000)-DAP (‘adhesion lipid’; DAP conjugated on the free ends of the PEG-chains), compositions numbered 3 and 4 on Table 1-2). Error bars correspond to standard deviations of repeated measurements (n=2 independent liposome preparations, 2 samples per preparation). Scale bar on confocal images corresponds to 10 μm. Liposomes were incubated with cells in suspension at the ratio of 10⁶ liposomes per cell (0.2 mM total lipid and 0.8×10⁶ cells per mL) for 3 hours. % cell-associated liposomes=(fluorescence of liposomes associated with given number of cells)/(fluorescence of all liposomes incubated with the same number of cells)×100. A-C: Please notice the different scale on the y-axis;

FIG. 4A and FIG. 4B show time-integrated intraspheroid distributions (FIG. 4A) of the lipid concentrations, and (FIG. 4B) of the concentrations of the fluorescent surrogate of cisplatin CFDA delivered by different types of liposomes. Liposomes with different combinations of the release (R) and the adhesion (A) properties are indicated as follows: R+A+(gray half-filled circles), R+A− (gray circles), R−A+(white half-filled circles), R−A− (white circles). Error bars correspond to standard deviations of measurements of n=3-5 spheroids per liposome composition per time point;

FIG. 5A, FIG. 5B, FIG. 5C, and FIG. 5D show: Left panel. Growth control of multicellular spheroids following treatment with cisplatin encapsulated in liposomes with the following combinations of the release (R) and adhesion (A) properties: R+A+(gray half-filled circles), R+A− (gray circles), R−A+(white half-filled circles), R−A− (white circles). Non-treated spheroids are indicated by a thick dashed line, and spheroids treated with free cisplatin by filled circles. Right panel. Extent of outgrowth of spheroids following the end time point shown in the plots on the left panel. Pattern and colors agree with constructs shown on left; non-treated spheroids are shown in white bars with thick black diagonal pattern. (FIG. 5A) MDA-MB-436 (ATCC) spheroids treated with 35 μM of cisplatin in all forms, (FIG. 5B) MDA-MB-231 (ATCC) spheroids treated with 150 μM cisplatin, (FIG. 5C) MDA-MB-231 (LUNG1) spheroids treated with 150 μM cisplatin, (FIG. 5D) MDA-MB-231 (ALN2) treated with 150 μM cisplatin. The concentration of cisplatin incubated with spheroids of each cell type was chosen to agree with the IC₅₀ value of the corresponding cells in monolayers. Error bars correspond to standard deviations of repeated measurements: two independent runs were performed with a total of 14-16 spheroids per construct. ** indicates p-values<0.01. * indicates p-values 0.01<p<0.05. Percent change in volume=Vₜ/Vₒ×100%, where Vₜ is volume at time t and Vₒ volume before initiation of treatment. At t=0 the average diameter of spheroids was 300±50 μm;

FIG. 6A, FIG. 6B, FIG. 6C, and FIG. 6D show uptake and clearance kinetics of ¹¹¹In-DTPA loaded liposomes (without the release property) following i.v. administration in mice bearing orthotopic MDA-MB-231 xenografts. (FIG. 6A) tumor, (FIG. 6B) blood pool, (FIG. 6C) liver, (FIG. 6D) spleen. Liposomes with the adhesion property (half-filled circles); liposomes without the adhesion property (open circles). Error bars correspond to standard errors of measurements averaged over n=3 mice per time point. * p-value=0.006, †−p-value=0.104;

FIG. 7 shows growth rates of the spontaneous MDA-MB-231 axillary lymph node (ALN) metastases in mice treated with cisplatin encapsulated in liposomes with the following combinations of the release (R) and adhesion (A) properties: R+A+(gray half-filled circles), R+A− (gray circles), R−A+(white half-filled circles), R−A− (white circles). Approximately 2.5 weeks following complete resection of the orthotopic MDA-MB-231 xenografts, animals were administered i.v., three times in five-day intervals, 7.5 mg/kg cisplatin. Growth rates on non-treated animals are shown by white squares. Treatment groups consisted of 5-7 animals. Error bars correspond to standard deviations of the growth rates of metastases in individual animals (shown in FIG. 16). *p-value<0.05, ** p-value<0.01;

FIG. 8A, FIG. 8B, and FIG. 8C show the chemical characterization of the presently disclosed ‘adhesion lipid’ DPPE-PEG(2000)-DAP. (FIG. 8A) TLC experimental conditions and (FIG. 8B) results of custom molecule indicating purity >99%. For TLC, the standard used was 18:1 PE with 1% 10:1 Lyso PE and 1% 18:1 fatty acid, with mobile phase 80:20:1 chloroform:methanol:ammonium hydroxide. TLC-based >99% reported purity was determined by comparison to a known mixture of 1% ‘impurity’ plated side-by-side with the DPPE-PEG(2000)-DAP lipid. (FIG. 8C) The certificate of analysis (on file, available upon request) using Phosphorus and Proton NMR characterization of the adhesion lipid reports the molecular weight of 2889.62 and molecular formula C₁₃₇H₂₇₅N₄O₅₅P as consistent with structure;

FIG. 9 shows the location of titratable cationic charge (DAP) relative to the lipid membrane affects the clearance kinetics of liposomes from the extracellular matrix of decellularized MDA-MB-231 orthotopic tumor xenografts. (white circles) Liposomes without charge containing only DSPE-PEG(2000); (half-red/half-white circles) Liposomes containing DAP conjugated on the free ends of the PEG-chains, DSPE-PEG(2000)-DAP; (red circles) Liposomes containing DAP located directly on the lipid headgroups, DSPE-DAP. Errors correspond to n=4 independent tumor samples and liposome preparations;

FIG. 10A, FIG. 10B, FIG. 10C, and FIG. 10D show the dose response (IC₅₀ plots) of MDA-MB-231 (ATCC) monolayers to different types of liposomal cisplatin (CDDP) after a 6-hour incubation. Black symbols: pH 7.4, grey symbols: pH 6.5. Filled symbols: liposomes loaded with cisplatin (CDDP), open symbols: empty liposomes. Liposomes with different combinations of the release (R) and the adhesion (A) properties were studied: (FIG. 10A) R+A+, (FIG. 10B) R+A−, (FIG. 10C) R−A+, (FIG. 10D) R−A−. Errors correspond to standard deviations of three independent liposome preparations;

FIG. 11A, FIG. 11B, FIG. 11C, and FIG. 11D show the dose response (IC₅₀ plots) of MDA-MB-436 (ATCC) monolayers to different types of liposomal cisplatin (CDDP) after a 6-hour incubation. Black symbols: pH 7.4, grey symbols: pH 6.5. Filled symbols: liposomes loaded with cisplatin (CDDP), open symbols: empty liposomes. Liposomes with different combinations of the release (R) and the adhesion (A) properties were studied: (A) R+A+, (B) R+A−, (C) R−A+, (D) R−A−. Errors correspond to standard deviations of three independent liposome preparations;

FIG. 12A, FIG. 12B, FIG. 12C, FIG. 12D, and FIG. 12E show interstitial pH gradients (pHₑ) of 300 μm-in-diameter spheroids determined by the cell-membrane impermeant, pH-indicator SNARF-4F. (FIG. 12A) Spheroids formed of MDA-MB-436 (ATCC) cells, (FIG. 12B) MDA-MB-231 (ATCC), (FIG. 12C) MDA-MB-231 (PRI3) cells, (FIG. 12D) MDA-MB-231(LUNG1) cells, and (FIG. 12E) MDA-MB-231 (ALN2) cells. Errors correspond to standard deviations of the measurements on different spheroids (n=5-6);

FIG. 13A and FIG. 13B show liposomes without the adhesion property (FIG. 13A) clear faster from the interstitium of spheroids compared to liposomes with the adhesion property (FIG. 13B) as indicated by the red bracket. Black symbols correspond to the measured distributions right after 6 hours of incubation with the corresponding liposomes. Gray symbols correspond to measured distributions 1 hour after removal of liposomes from the surrounding solution. Error bars correspond to standard deviations of measurements of n=3-5 spheroids per liposome composition per time point;

FIG. 14A, FIG. 14B, FIG. 14C, and FIG. 14D show the extent of outgrowth of spheroids following treatment with empty liposomes (liposomes not containing cisplatin) having the following combinations of the release (R) and adhesion (A) properties: R+A+(gray half-filled circles), R+A− (gray circles), R−A+(white half-filled circles), R−A− (white circles). Non-treated spheroids are shown in white bars with thick black diagonal pattern. Spheroids formed by (FIG. 14A) MDA-MB-436 (ATCC) cells, (FIG. 14B) MDA-MB-231 (ATCC) cells, (FIG. 14C) MDA-MB-231 (LUNG1) cells, (FIG. 14D) MDA-MB-231 (ALN2) cells. Error bars correspond to standard deviations of repeated measurements: two independent runs were performed with a total of 14-16 spheroids per construct. At t=0 the average diameter of spheroids was 300±50 μm;

FIG. 15A, FIG. 15B, and FIG. 15C show the uptake and clearance kinetics of ¹¹¹In-DTPA loaded liposomes following i.v. administrations in mice bearing orthotopic MDA-MB-231 xenografts. (FIG. 15A) Heart, (FIG. 15B) kidneys, (FIG. 15C) lungs. Liposomes with the adhesion property (half-filled circles); liposomes without the adhesion property (open circles). Error bars correspond to standard errors of measurements averaged over n=3 mice per time point;

FIG. 16 shows the growth over time of the volume of spontaneous MDA-MB-231 axillary lymph node (ALN) metastases in individual mice following, on average, 2.5 weeks upon complete resection of the orthotopic xenografts. The volume of metastases was monitored and quantified by MRI. Animals were administered i.v., three times in five-day intervals (day 0, 5 and 10), 7.5 mg/kg cisplatin in liposomal and in freeform and were scanned once per week. The slope of the fitted linear function was used as the growth rate of the ALN metastases and was averaged per treatment group. Liposomes with different combinations of the release (R) and the adhesion (A) properties are indicated as follows: R+A+(gray half-filled circles), R+A− (gray circles), R−A+(white half-filled circles), R−A− (white circles). The non-treated group is indicated by white squares.;

FIG. 17 shows the growth over time of the volume of spontaneous MDA-MB-231 axillary lymph node (ALN) metastases in individual mice treated with free cisplatin at its reported MTD (7.5 mg/Kg). The volume of metastases was monitored and quantified by MRI. Animals were administered i.v., three times in five-day intervals (day 0, 5 and 10), and were scanned once per week. The last volume measurement on each animal indicates the day of sacrifice, as well;

FIG. 18A, FIG. 18B, FIG. 18C, FIG. 18D, and FIG. 18D show H&E stained sections of the organs of tumor-bearing mice indicating tumor emboli (or tumor circulating cells, in B) in several normal organ sites. Scale bar is (FIG. 18A) 0.2 mm, (FIG. 18B) 500 μm, (FIG. 18C) 200 μm, (FIG. 18D) 50 μm, (FIG. 18E) 500 μm;

FIG. 19A and FIG. 19 B are a colony survival assay for MDA-MB-231 TNBC cells following six hours incubation at two pH values (7.4 in FIG. 19A, and 6.0 in FIG. 19B) with different radioactivity concentrations of ²²⁵Ac-DOTA in free form (black circles) and ²²⁵Ac-DOTA delivered by liposomes with the following combinations of the release (R) and adhesion (A) properties: R+A+(gray half-filled circles), R+A− (gray circles), R−A+(white half-filled circles), R−A− (white circles). Values are the averages and standard deviations of n=3 independent liposome preparations;

FIG. 20A, FIG. 20B, and FIG. 20C show: FIG. 20A, volume growth control of multicellular spheroids following treatment with 9 kBq/mL of ²²⁵Ac-DOTA delivered by liposomes with the following combinations of the release (R) and adhesion (A) properties: R+A+(gray half-filled circles), R+A− (gray circles), R−A+(white half-filled circles), R−A− (white circles). Non-treated spheroids are indicated by a thick dashed line.

FIG. 20B, characteristic images of spheroids from different treatment groups. FIG. 20C, extent of outgrowth of spheroids (used as indirect surrogate of tumor recurrence) following the end time point shown in the plots on the left panel. Pattern and colors agree with constructs shown on left. Error bars correspond to standard deviations of repeated measurements: four independent spheroid preparations and liposome preparations were performed with a total of 14-16 spheroids per construct. ** indicates p-values<0.01. * indicates p-values 0.01<p<0.05. Percent change in volume=Vₜ/Vₒ×100%, where Vₜ is volume at time t and Vₒ volume before initiation of treatment. At t=0 the average diameter of spheroids was 400±40 μm;

FIG. 21 shows tumor pHₑ maps imaged by MRI confirm acidity in the tumor interstitum that is necessary to trigger the release of ²²⁵Ac-DOTA from liposomes. Tumor-bearing animals were anesthetized, positioned in the magnet isocenter, and 0.4 mL of 1 M ISUCA (Imidazole Succinic Acid sodium salt; blue solution) was injected I.P.. Successive multivoxel spectroscopy grids were acquired, the Henderson-Hasselbalch calibration curve was generated, and the measured ISUCA chemical shift in every voxel was transformed into an extracellular pH value generating a pHₑ map. Pacheco-Torres et al., 2015;

FIG. 22A shows volume change over time of orthotopic MDA-MB-231 TNBC tumors upon I.V. administration of a single dose of 4.625 kBq (125 nCi) per 20 gr NSG mouse of ²²⁵Ac-DOTA delivered by liposomes with different combinations of release and adhesion properties. The greatest inhibition of the orthotopic xenograft growth was achieved by those carriers bearing both the release and the adhesion properties (R+A+; p-value<0.01). Mice were treated with ²²⁵Ac-DOTA delivered by liposomes with the following combinations of the release (R) and adhesion (A) properties: R+A+(gray half-filled circles), R+A− (gray circles), R−A+(white half-filled circles), R−A− (white circles);

FIG. 22B shows the percentage of animals with metastases in day 14 after administration of radiotherapy, animals were euthanized and were imaged by MM to detect formation of spontaneous ALN metastases. ²²⁵Ac-DOTA loaded liposomes with both properties (R+A)—interstitial release and adhesion to ECM—completely eliminated the appearance of spontaneous metastases at the time point of observation. Pattern and colors agree with constructs shown on FIG. 22A. ** indicates p-values<0.01. * indicates p-values 0.01<p<0.05;

FIG. 23A demonstrates that with lowering pH, liposomes release faster and more extensively the encapsulated cisplatin. The rates and extents of cisplatin released from pH-releasing liposomes are tabulated below. Liposomes were loaded with cisplatin at neutral pH (as described in the Methods section), and then they were introduced in solutions of acidic pH. Samples from the parent liposome suspension were removed at different time points, released cisplatin was separated from the liposomal cisplatin by size exclusion chromatography, and the amount of cisplatin on different fractions was measured using AAS (as described in Methods section). Errors correspond to standard deviations of six independent liposome preparations;

FIG. 23B shows the mechanism of content release from liposomes. Supporting to the findings suggesting a “all-or-none” release mechanism from liposomes, described on the paragraph below, is Figure S 11-B which shows that gradual acidification of the same liposome suspension (from pH 7.4 to 6.5, and then from 6.5 to pH 5.5), reveals a potentially additional population of liposomes that releases its contents at a lower pH. In the current study, the adhesion property on these liposomes may prolong their residence times within tumors and, may potentially increase the probability that these liposomes experience microenvironments with more acidic pH so as to collectively release even more of their therapeutic contents;

FIG. 23C and FIG. 23D support the “all-or-none” release mechanism. Liposomes in neutral pH loaded with self-quenching concentrations of calcein (ranging from 55 mM to 10 mM) exhibiting maximum self-quenching efficiencies ranging from 11 to 2 (where Qₘₐₓ=[fluorescence intensity after relief of self-quenching by Triton X-100]/[fluorescence intensity before addition of Triton X-100]), were introduced in solutions of gradually lower pH. At each pH value, the extent of content released was monitored in time, and when the fraction of content released reached an asymptotic value, the quenching efficiency of the liposomal suspension was measured again (and was lower than the initial Qmax since calcein had been released from the liposomes, indicated by the black symbols). Following this measurement, the liposome suspensions were passed through a size exclusion chromatography column to remove the released calcein, and the freshly eluted liposome suspension was measured again for its quenching efficiency (white symbols) which was comparable to the original maximum quenching efficiency Qmax that was measured on liposome suspensions formed in neutral pH. The process was repeated by lowering further the pH of the liposome suspension and ultimately again measuring the quenching efficiency of liposomes after SEC which was close to the initial Qₘₐₓ. These findings support the “all-or-none” release mechanism; i.e. at each given pH, a population of liposomes will either release their entire amount of encapsulated contents or will not release their contents at all. Errors correspond to standard deviations of two independent liposome preparations.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Adhesive/Adsorption Switch on Nanoparticles to Increase Tumor Uptake and Delay Tumor Clearance

The presently disclosed subject matter provides lipid vesicles for drug delivery is which the lipid vesicles adhere on cells and avoid internalization by cells. The presently disclosed subject matter demonstrates that it is the proximity of the cationic charge to the lipid membrane of the charge-bearing lipid vesicles that (following the initial adhesion) may be critical in affecting the extent of fusion and/or endocytosis of the vesicles by living cells. In general, the presently disclosed subject matter provides lipid vesicles with grafted PEG-chains in which the distance of the cationic charge relative to the plane of the vesicle headgroups is varied. Vesicles with the cationic charge directly on the lipid headgroups or on the free PEG-chain ends were compared. Lipid vesicles with the cationic charge directly on the lipid headgroups interact very differently with the apposing living cell membranes from lipid vesicles with the cationic charge on the ends of PEG-chains and the same zeta potential. Additionally, on the drug delivery end, the presently disclosed subject matter demonstrates that the primary effect of the cationic charge when on the end of undulating PEG-chains is to obstruct cell internalization of lipid vesicles and to delay their clearance from solid tumors in vivo. Overall, the location of electrostatic charges on lipid vesicles can be used as a tool to precisely tune the interactions of lipid vesicles with living cells with implications in drug delivery and therapy.

The efficacy of tumor-delivered doses of one or more therapeutic agents can be enhanced when delivered by carriers that improve the uniformity in intratumoral drug distributions. Minchinton and Tannock, 2006. It has been previously demonstrated in 3D multicellular spheroids (used as surrogates of the tumor avascular regions) that this goal can be facilitated by nanocarriers engineered to release their (rapidly diffusing) therapeutic contents in the tumor interstitium enabling deep tumor-penetration of therapeutics. Stras et al., 2016; Zhu et al., 2017. One key to this approach is to use drug nanocarriers that do not become internalized by cells so as to maximize the fraction of released drug that may penetrate deeper in the tumor and, to choose therapeutic agents which are efficiently transported across the cell membranes independent of the local extracellular milieu.

To effectively translate this strategy in vivo, however, the intratumoral residence times of such drug-loaded nanocarriers should be increased to maximize the time-integrated dose delivered at the tumor. To increase the nanocarriers' tumor residence times, the presently disclosed subject matter introduces an ‘adsorptive/adhesive switch’ on the nanocarriers' surface with the aim to slow down their tumor-clearing kinetics. The switch is designed to promote nanoparticle adsorption on cancer cells and/or the ECM, while keeping their internalization by cells to a minimum.

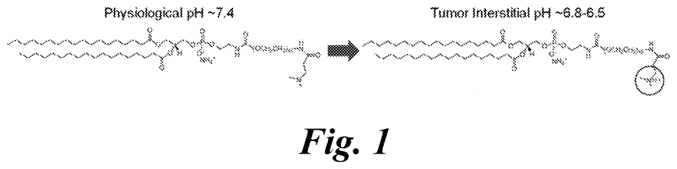

The adsorptive switch, which is an electrostatic switch attributing positive charge on the liposome corona and thereby increasing the liposomes' tendency to adsorb on cells and the ECM, Lieleg et al., 2009; Stylianopoulos et al., 2010, is introduced, in some embodiments, by the chemical moiety dimethyl ammonium propane (DAP). In particular embodiments as provided herein, DAP can be conjugated on the free end of PEG, which is used in the form of PEGylated lipids. See FIG. 1.

The intrinsic pKa of free DAP is approximately 6.7, Auguste et al., 2006, which is comparable to the pH values in the tumor acidic interstitium. The design of this switch is based on the rationale that during liposome circulation, the PEGylated corona of the nanocarrier would not be positive, and, therefore, liposomes would exhibit a low tendency to adsorb on anionic surfaces. Upon tumor internalization and liposome diffusion toward the tumor acidic interstitium, protonation of the DAP-PEG-lipids would attribute a cationic charge on the liposome PEGylated corona, potentially increasing their adsorption to anionic surfaces, namely the cells and the ECM.

Contrary to previously reported DAP-containing liposomes and other particles, the titratable charge was designed to be located on the edge of the PEG corona and not conjugated on the lipid headgroups; the latter usually promotes electrostatic adsorption of liposomes on the cell plasma membrane and may result in lipid fusion with the plasma membranes and cellular internalization of liposome contents. The presently disclosed surface architecture, where the charge is localized on the free end of PEG chains grafted on liposomes, was designed to increase adhesion/adsorption on cells, to minimize the internalization of these liposomes by cells, and to effectively delay liposome clearance from the tumors because of their electrostatic adsorption on extracellular compartments within the tumor and not because of their internalization by cells.

More particularly, the presently disclosed adhesive switch is designed to attribute positive charge on the lipid nanoparticle corona in the slightly acidic pH of the tumor interstitium. Helmlinger et al., 1997; Vaupel et al., 1989. In representative embodiments, its molecular structure involves the moiety dimethyl ammonium propane (DAP) conjugated on the free PEG-chain end of PEG-lipids (see FIG. 1) with an intrinsic pKa of about 6.7. Auguste et al., 2006. The presently disclosed subject matter demonstrates, in part, that such lipid nanoparticles adhere on cells when the extracellular pH is acidified, then desorb from cells when the extracellular pH is raised back to physiological values, and do not fuse with the cell membranes.

The primary effect of the presently disclosed adhesive switch is to increase tumor uptake and to delay the tumor clearance kinetics in vivo, without affecting the blood clearance kinetics of NPs. Contrary to previously reported cationic liposomes, Bailey and Cullis, 1997, which bear the charge on their lipid-headgroup surface, Sokolova et al., 2013; Lin and Alexander-Katz, 2013, the presently disclosed design allows for adhesion of nanoparticles to cells but does not support their internalization by cells.

Further, the adhesion property of the presently disclosed nanoparticles results in greater tumor uptake of the nanoparticles and in slower clearance of NPs from tumors in vivo without affecting the blood clearance kinetics (relative to nanoparticles of the same size and PEGylation). The strong interaction of the presently disclosed adhesive nanoparticles with the ECM of these tumors seems to play a central role in the delayed clearance from tumors. As provided in more detail herein below, in vivo, the presently disclosed nanoparticles with the adhesion property exhibit greater tumor uptake, slower tumor clearance and greater AUCₜᵤₘₒᵣ compared to same size NPs without the adhesion switch. The adhesion property does not change the blood circulation kinetics of the nanoparticles. The presently disclosed adhesive nanoparticles are cleared significantly more slowly (τ_{1/2}=95 vs. 65 min), Stras and Sofou, 2018, unpublished results, than non-adhering nanoparticles from the ECM extracted (by decellularization), Lü et al., 2014, from breast cancer tumors.

More particularly, in some embodiments, the presently disclosed subject matter provides lipid carriers for selective tumor delivery, due to their nanometer size, which are loaded with a therapeutic agent, e.g., cisplatin, and are designed to exhibit the following properties when in the tumor interstitium: 1) interstitial drug release (for deeper tumor penetration of cisplatin) and/or 2) intratumoral/interstitial adhesion of the carriers (not accompanied by cell internalization) for delayed tumor clearance and longer cancer cell exposure to cisplatin being released.

The presently disclosed subject matter demonstrates, in part, that on large multicellular spheroids, used as surrogates of avascular solid tumors' areas, greater efficacy was strongly correlated with more uniform and higher time-integrated concentrations of the delivered agents. Lipid nanocarriers with both the release and adhesion properties were more effective followed by nanocarriers with only the releasing property, and by nanocarriers with only the adhering property. In vivo, cisplatin-loaded nanocarriers with the releasing and/or the adhering properties significantly delayed the growth of spontaneous TNBC metastases, and the efficacy of different properties' combinations followed the same trends as in spheroids.

The presently disclosed subject matter demonstrates the therapeutic potential of a general strategy to bypass treatment limitations of TNBC metastases due to lack of cell-targeting markers, by aiming to optimize the spatiotemporal intratumoral drug distributions for more uniform and prolonged drug exposure.

Accordingly, in some embodiments, the presently disclosed subject matter provides a lipid-based nanocarrier of formula (I):

L-P—R₁ (I);

wherein: L is a phospholipid; P is a polyethylene glycol linker; and R₁ is a moiety having a tritratable cationic charge that becomes positively charged under a physiological pH of a tumor interstitium; wherein: R₁ is conjugated to a free end of the polyethylene glycol linker; the lipid-based nanocarrier adheres to a target cell or the extracellular matrix (ECM) thereof, and wherein internalization of the lipid-based nanocarrier by the target cell is minimized; and pharmaceutically acceptable salts thereof.

As used herein, a “lipid” is generally a biomolecule that is soluble in nonpolar solvents. A lipid can be hydrophobic or amphiphilic. The amphiphilic nature of lipids allows them to form vesicles or liposomes in an aqueous environment. Lipids generally can be classified into eight categories: fatty acids, glycerolipids, glycerophospholipids, sphingolipids, saccharolipids, and polyketides (derived from condensation of ketoacyl subunits); and sterol lipids and prenol lipids (derived from condensation of isoprene subunits). Lipids commonly comprise fatty acids or fatty acid residues.

Representative fatty acids include saturated fatty acids and non-saturated fatty acids. Saturated fatty acids do not have a carbon-carbon double bond have a general formula of CH₃(CH₂)ₙCOOH, wherein n can be an integer from about 4 to about 40 or more, including 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40. Common saturated fatty acids include, but are not limited to: butyric acid (butanoic acid, n=4), valeric acid (pentanoic acid, n=5), caproic acid (hexanoic acid, n=6), enanthic acid (heptanoic acid, n=7), caprylic acid (octanoic acid, n=8), pelargonic acid (nonanoic acid, n=9), capric acid (decanoic acid, n=10), undecylic acid (undecanoic acid, n=11), lauric acid (dodecanoic, n=12), tridecylic acid (tridecanoic acid, n=13), myristic acid (tetradecanoic acid, n=14), pentadecylic acid (pentadecanoic acid, n=15), palmitic acid (hexadecanoic acid, n=16), margaric acid (heptadecanoic acid, n=17), stearic acid (octadecanoic acid, n=18), nonadecylic acid (nonadecanoic acid, n=19), arachidic acid (eicosanoic acid, n=20), heneicosylic acid (heneicosanoic acid, n=21), behenic acid (docosanoic acid, n=22), tricosylic acid (tricosanoic acid, n=23), lignoceric acid (tetracosanoic acid, n=24), pentacosylic acid (pentacosanoic acid, n=25), cerotic acid (hexacosanoic acid, n=26), heptacosylic acid (heptacosanoic acid, n=27), montanic acid (octacosanoic acid, n=28), nonacosylic acid (nonacosanoic acid, n=29), melissic acid (triacontanoic acid, n=30), henatriacontylic acid (henatriacontanoic acid, n=31), lacceroic acid (dotriacontanoic acid, n=32), psyllic acid (tritriacontanoic acid, n=33), geddic acid (tetratriacontanoic acid, n=34), ceroplastic acid (pentatriacontanoic acid, n=35), henatriacontylic acid (hexatriacontanoic acid, n=36), heptatriacontanoic acid (n=37), octatriacontanoic acid (n=38), nonatriacontanoic acid (n=39), and tetracontanoic acid (n=40).

Unsaturated fatty acids include one or more carbon-carbon double bonds, for example one, two, or three carbon-carbon double bonds, and can include cis or trans isomers. Unsaturated fatty acids can have from about 4 carbon atoms to about 24 carbon atoms, including 4, 5, 6, 7, 8, 9, 10, 11, 12, 13, 14, 15, 16, 17, 18, 19, 20, 21, 22, 23, and 24 carbon atoms. Representative unsaturated fatty acids include, but are not limited to, mono-unsaturated fatty acids, including crotonic acid, myristoleic acid, palmitoleic acid, sapienic acid, oleic acid, elaidic acid, vaccenic acid, gadoleic acid, eicosenoic acid, erucic acid, and nervonic acid; di-unsaturated fatty acids, including, linoleic acid, eicosadienoic acid, and docosadienoic acid; tri-unsaturated fatty acids, including, linolenic acid, pinolenic acid, eleostearic acid, mead acid, dihomo-γ-linolenic acid, and eicosatrienoic acid; tetra-unsaturated fatty acids, including, stearidonic acid, arachidonic acid, eicosatetraenoic acid, and adrenic acid; pentaunsaturated fatty acids, bosseopentaenoic acid, eicosapentaenoic acid, ozubondo acid, sardine acid, and tetracosanolpentaenoic acid; and hexa-unsaturated fatty acids, including docosahexaenoic acid and herring acid.

As used herein, the term “lipsosome” generally refers to a spherical vesicle having at least one lipid bilayer. Liposomes commonly comprise phospholipids. A phospholipid generally consists of two hydrophobic fatty acid “tails” and a hydrophilic “head” consisting of a phosphate group. The two components are joined together by a glycerol molecule.

As used herein, the term “PEG” refers to polyethylene glycol, which has the following general structure:

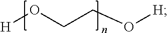

where n is an integer from 1 to 1000. The numbers that are included in the names of PEGs, e.g., PEG(2000) and the like, indicate their average molecular weights (e.g., a PEG with n=9 would have an average molecular weight of approximately 400 daltons, and would be labeled PEG(400)). Representative PEGs suitable for use with the presently disclosed subject matter include, but are not limited to, PEG(100), PEG(200), PEG(300), PEG(400), PEG(600), PEG(800), PEG(1000), PEG(1500), PEG(2000), PEG(3000), PEG(3350), PEG(4000), PEG(6000), PEG(8000), PEG(10,000), and PEG(35,000). In particular embodiments, the PEG is PEG(2000).

In particular embodiments, the lipid-base nanocarrier comprising a compound of formula (I) has the following structure:

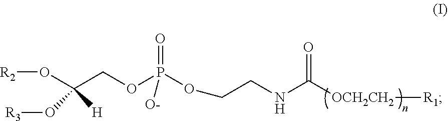

wherein: n is an integer from 1 to 1000; R₁ is a moiety having a tritratable cationic charge that becomes positively charged under a physiological pH of a tumor interstitium; R₂ and R₃ are each independently a fatty acid or fatty acid residue, wherein R₂ and R₃ can be the same or different; and pharmaceutically acceptable salts thereof.

In yet more particular embodiments, R₁ comprises a moiety having an intrinsic pKa having a range from about 6.0 to about 6.9, including 6.0, 6.1, 6.2, 6.3, 6.4, 6.5, 6.6, 6.7, 6.8, and 6.9. In yet more particular embodiments, R₁ is dimethyl ammonium propane.

In certain embodiments, the compound of formula (I) has the following formula:

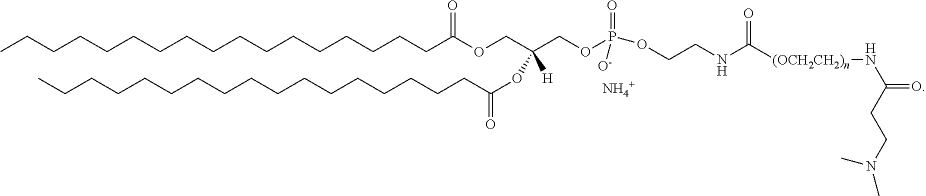

In other embodiments, the lipid-based nanocarrier further comprises one or more therapeutic agents. In particular embodiments, the one or more therapeutic agents comprises a chemotherapeutic agent. In some embodiments, the therapeutic agent comprises a radionuclide, such as an alpha-particle emitter for internal radiotherapy. In particular embodiments, the alpha-particle emitter is 225-Actinium.

In some embodiments, the lipid-based nanocarrier further comprises one or more chelating agents. In particular embodiments, the chelating agent is selected from the group consisting of DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethyl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and BaBaSar.

In more particular embodiments, the chelating agent is selected from the group consisting of:

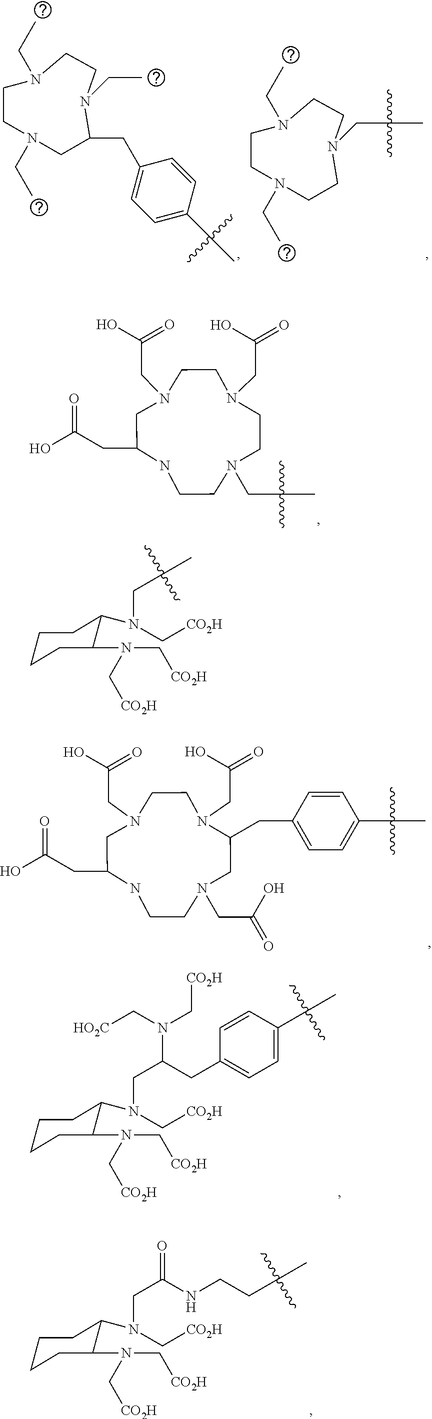

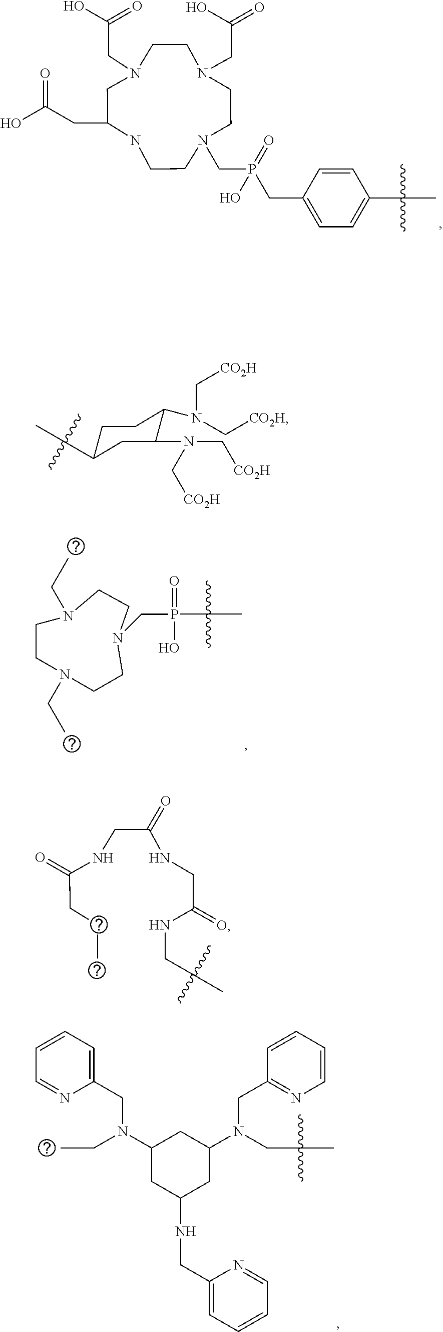 [SMILES: *c1ccc(CP(=O)(O)CN2CCNC(CC(=O)O)CN(CC(=O)O)CCN(CC(=O)O)CC2)cc1 ; *C1CCC(N(CC(=O)O)CC(=O)O)C(N(CC(=O)O)CC(=O)O)C1 ; *P(=O)(O)CN1CCN(CC)CCN(CC)CC1 ; *CNC(=O)CNC(=O)CNC(=O)CCC ; *CN(Cc1ccccn1)C1CC(NCc2ccccn2)CC(N(CC)Cc2ccccn2)C1]

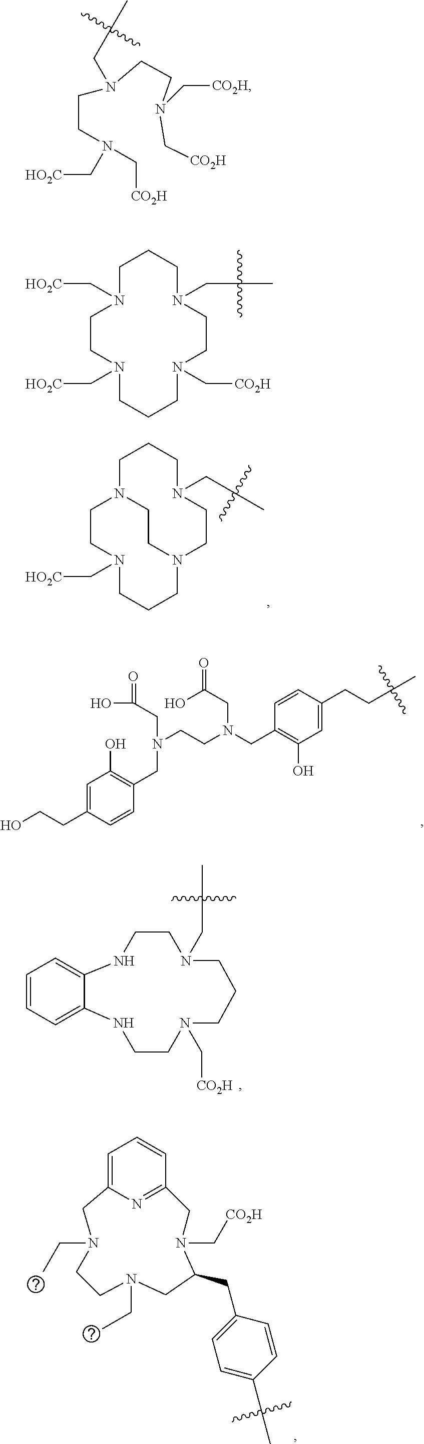

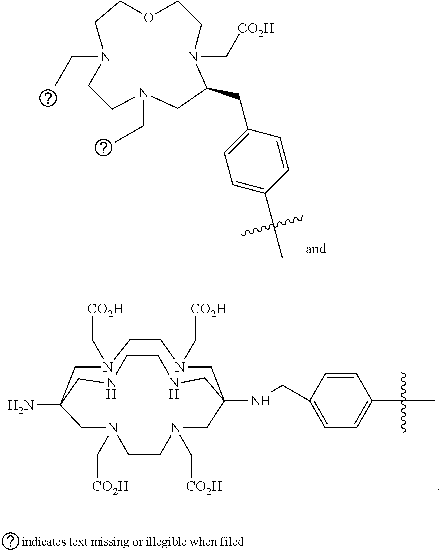 [SMILES: *c1ccc(C[C@H]2CN(CC)CCN(CC)CCOCCN2CC(=O)O)cc1 ; *c1ccc(CNC23CNCCNCC(N)(CN(CC(=O)O)CCN(CC(=O)O)C2)CN(CC(=O)O)CCN(CC(=O)O)C3)cc1]

In certain embodiments, the chelating agent comprises a radiometal selected from the group consisting of: ^{94m}Tc, ^{99m}Tc, ¹¹¹In, ⁶⁷Ga, ⁶⁸Ga, ⁸⁶Y, ⁹⁰Y, ¹⁷⁷Lu, ¹⁸⁶Re, ¹⁸⁸Re, ⁶⁰Cu, ⁶¹Cu, ⁶²Cu, ⁶⁴Cu, ⁵⁵Co, ⁵⁷Co, ⁴⁷Sc, ²²⁵Ac, ²¹³Bi, ²¹²Bi, ²¹²Pb, ¹⁵³Sm, ¹⁶⁶Ho, ¹⁵²Gd, ⁸²Rb, ⁸⁹Zr, and ¹⁶⁶Dy.

In some embodiments, the chemotherapeutic agent is an alkylating agent, including cyclophosphamide, mechlorethamine, chlorambucil, melphalan, and dacarbazine; a nitrosoureas, including temozolomide (oral dacarbazine); an anthracycline, including daunorubicin, doxorubicin, epirubicin, idarubicin, mitoxantrone, and valrubicin; a cytoskeletal disruptors (taxanes), including paclitaxel, docetaxel, abraxane, and taxotere; epothilones; histone deacetylase inhibitors, including vorinostat, and romidepsin; inhibitors of topoisomerase I, including irinotecan and topotecan; inhibitors of topoisomerase II, including etoposide, teniposide, and tafluposide; kinase inhibitors, including bortezomib, erlotinib, gefitinib, imatinib, vemurafenib, and vismodegib; nucleotide analogs and precursor analogs, including azacytidine, azathioprine, capecitabine, cytarabine, doxifluridine, fluorouracil, gemcitabine, hydroxyurea, mercaptopurine, methotrexate, and tioguanine; antimicrobial peptides, including bleomycin and actinomycin; platinum-based agents, including platinum-based antineoplastics, such as cisplatin, carboplatin, oxaliplatin, nedaplatin, triplatin tetranitrate, phenanthriplatin, picoplatin, and satraplatin; retinoids, including, tretinoin, alitretinoin, and bexarotene; and vinca alkaloids and derivatives, including vinblastine, vincristine, vindesine, and vinorelbine.

In particular embodiments, the chemotherapeutic agent is selected from the group consisting of actinomycin, all-trans retinoic acid, azacytidine, azathioprine, bleomycin, bortezomib, carboplatin, capecitabine, cisplatin, chlorambucil, cyclophosphamide, cytarabine, daunorubicin, docetaxel, doxifluridine, doxorubicin, epirubicin, epothilone, etoposide, fluorouracil, gemcitabine, hydroxyurea, idarubicin, imatinib, irinotecan, mechlorethamine, mercaptopurine, methotrexate, mitoxantrone, oxaliplatin, paclitaxel, pemetrexed, teniposide, tioguanine, topotecan, valrubicin, vemurafenib, vinblastine, vincristine, and vindesine.

In other embodiments, the presently disclosed subject matter provides a method for treating a disease, disorder, or condition, the method comprising administering a therapeutically effective amount of a lipid-based nanocarrier of Formula (I), or a pharmaceutical formulation thereof, to a subject in need of treatment thereof.

In particular embodiments, the disease, disorder, or condition comprises a cancer. In more particular embodiments, the cancer comprises a metastatic cancer. In yet more particular embodiments, the cancer is selected from the group consisting of testicular cancer, ovarian cancer, cervical cancer, breast cancer, bladder cancer, head and neck cancer, esophageal cancer, lung cancer, mesothelioma, brain tumors, and neuroblastoma. In even yet more particular embodiments, the cancer is breast cancer. In certain embodiments, the breast cancer is triple negative breast cancer (TNBC).

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

In general, the “effective amount” of an active agent refers to the amount necessary to elicit the desired biological response. As will be appreciated by those of ordinary skill in this art, the effective amount of an agent may vary depending on such factors as the desired biological endpoint, the agent to be delivered, the composition of the encapsulating matrix, the target tissue, and the like. As used herein, a “dose” refers to the amount of the presently disclosed lipid nanocarrier administered to a subject that is sufficient to treat the subject for a disease, disorder, or dysfunction.

In yet other embodiments, the presently disclosed subject matter provides a pharmaceutical formulation comprising a lipid-based nanocarrier of Formula (I) and a pharmaceutically acceptable carrier.

In the various embodiments described above, the presently disclosed lipid-based nanocarrier can be administered in a variety of forms depending on the desired route and/or dose. The presently disclosed lipid-based nanocarrier can be administered in a pharmaceutically acceptable carrier. As used herein, “pharmaceutically acceptable carrier” is intended to include, but is not limited to, water, saline, dextrose solutions, human serum albumin, liposomes, hydrogels, microparticles and nanoparticles.

Depending on the specific conditions being treated, the presently disclosed lipid-based nanocarrier may be formulated into liquid or solid dosage forms and administered systemically or locally. The agents may be delivered, for example, in a timed- or sustained-low release form as is known to those skilled in the art. Techniques for formulation and administration may be found in Remington: The Science and Practice of Pharmacy (20th ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

While the form and/or route of administration can vary, in some embodiments the presently disclosed lipid-based nanocarrier or pharmaceutical composition is administered parenterally (e.g., by subcutaneous, intravenous, or intramuscular administration), or in some embodiments is administered directly to the lungs. Local administration to the lungs can be achieved using a variety of formulation strategies including pharmaceutical aerosols, which may be solution aerosols or powder aerosols. Powder formulations typically comprise small particles. Suitable particles can be prepared using any means known in the art, for example, by grinding in an airjet mill, ball mill or vibrator mill, sieving, microprecipitation, spray-drying, lyophilization or controlled crystallization. Typically, particles will be about 10 microns or less in diameter. Powder formulations may optionally contain at least one particulate pharmaceutically acceptable carrier known to those of skill in the art. Examples of suitable pharmaceutical carriers include, but are not limited to, saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. Alternatively, solution aerosols may be prepared using any means known to those of skill in the art, for example, an aerosol vial provided with a valve adapted to deliver a metered dose of the composition. Where the inhalable form of the active ingredient is a nebulizable aqueous, organic or aqueous/organic dispersion, the inhalation device may be a nebulizer, for example a conventional pneumatic nebulizer such as an airjet nebulizer, or an ultrasonic nebulizer, which may contain, for example, from 1 to 50 mL, commonly 1 mL to 10 mL, of the dispersion; or a hand-held nebulizer which allows smaller nebulized volumes, e.g. 10 μL to 100 μL.

For injection, the agents of the disclosure may be formulated and diluted in aqueous solutions, such as in physiologically compatible buffers such as Hank's solution, Ringer's solution, or physiological saline buffer.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances such as, saline, preservatives, such as benzyl alcohol, absorption promoters, and fluorocarbons.

The “subject” treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein. The term “subject” also refers to an organism, tissue, cell, or collection of cells from a subject.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration and are not to be construed as limiting in any manner to make compounds of the disclosure by other methods.

### Example 1

### Adhesive/Adsorption Switch on Nanoparticles to Increase Tumor Uptake and Delay Tumor Clearance

### 1.1 Background

Triple Negative Breast Cancer (TNBC) accounts for 10-20% of breast carcinomas with the lowest 5-year survival rates among all breast cancer patients, Ovcaricek et al., 2011, due to high proliferation and reoccurrence outside the breast. Dawood, 2010; Dent et al., 2007. Metastatic TNBC is currently incurable. The poor prognosis, Dawood, 2010; Dent et al., 2007, in metastatic TNBC is attributed largely to the lack of tumor selective therapeutic modalities that effectively deliver lethal doses at the sites of disease. Fantini et al., 2012. A therapeutic modality that improves efficacy at the sites of TNBC metastases by better controlling tumor growth, without increasing toxicities, could have a significant clinical impact.

TNBC tumors frequently show sensitivity, Dawood, 2010; Anders et al., 2013, to platinum-derived agents, Telli, 2014, which have received extensive clinical use because of their DNA damaging activity. In the clinic, combination of platinum agents with experimental receptor-mediated targeted therapies designed to affect or inhibit key signaling pathways, Crown et al., 2012; Oakman, 2010, did not demonstrate statistically significant improvement following single-agent targeting approaches. Gelmon, 2012. Of lower toxicities but not yet with a significant improvement in therapeutic effect were also the clinical results of liposomal cisplatin (CDDP). Liu et al., 2013; van Hennik et al., 1987.

A strategy to increase the efficacy of delivered doses to TNBC metastases could aim at (1) improving uniformity in intratumoral drug distributions, Minchinton and Tannock, 2006, and (2) prolonging exposure of these cancer cells to delivered therapeutics. It has previously been demonstrated in 3D multicellular spheroids (used as surrogates of the tumor avascular regions) that improved intratumoral uniformity can be enabled by drug nanocarriers engineered to release their (rapidly diffusing, due to small size,) therapeutic contents in the tumor interstitium enabling deep tumor-penetration of therapeutics. Stras et al., 2016; Zhu et al., 2017. Key to this approach was shown to be the choice of drug nanocarriers that do not become internalized by cells—so as to maximize the fraction of released drug that may penetrate deeper in the tumor—and, to choose therapeutic agents (for example, cisplatin) which are efficiently transported across the cell membranes independent of the acidity of the local extracellular milieu. Stras et al., 2016.

To effectively translate this strategy in vivo, however, the intratumoral residence times of such drug-loaded nanocarriers need to be prolonged to maximize the time-integrated dose delivered at the tumor. In the presently disclosed subject matter, to increase the nanocarriers' tumor residence times, an ‘adhesion switch’ is introduced on the nanocarriers' surface with the aim to slow down their tumor clearing kinetics. The switch is designed to promote nanoparticle adhesion on the extracellular matrix (ECM) and/or on cancer cells while keeping their internalization by cells at a minimum.

Disclosed herein are lipid-based nanocarriers (liposomes) loaded with cisplatin and exhibiting interstitial drug release and intratumoral adhesion. Both mechanisms affecting these properties were designed to be activated in the slightly acidic pH of the tumor interstitium (pH about 6.7 to about 6.0). Helmlinger et al., 1997; Vaupel et al., 1989.

In particular, for the tumor interstitial release, lipid-based nanocarriers were designed to contain pH-responsive lipid membranes forming reversible phase-separated lipid domains (resembling lipid patches) with lowering pH, as was reported previously. Bandekar et al., 2012; Karve et al. 2009; Karve et al., 2008; Karve et al., 2010.

During circulation in the blood, these lipid-based nanocarriers comprise well-mixed, uniform membranes and stably retain their encapsulated contents. In the acidic tumor interstitium, occurrence of lipid-phase separation results in formation of lipid patches that span both lipid leaflets (cross-bilayer registration). Bandekar and Sofou, 2012.

It has been demonstrated that this lipid rearrangement in the bilayer membrane can be utilized to create pronounced grain boundaries around the lipid domains enabling release of the encapsulated therapeutic agents which then—in a drug delivery setting—may diffuse deeper into solid tumors. Stras et al., 2016; Zhu et al., 2017.

At the molecular level, lipid phase separation is enabled by balancing the permanent hydrogen-bonding attraction with the pH-tunable electrostatic repulsion between the lipids that form the domains (lipids with phosphatidyl serine headgroups, in the presently disclosed subject matter). The extent of membrane permeability on phase-separated bilayers was previously shown to be affected by the order of transient defects in the packing of lipid acyl-tails along the domain boundaries. Packing discontinuities along these boundaries may be enhanced by incorporation of saturated, gel-phase lipids with acyl-tails of different lengths. Karve et al., 2008.

The adhesion property—which is based on an electrostatic switch—attributing positive charge on the liposome corona—and, therefore, increasing the liposomes' tendency to adhere on the ECM, Lieleg et al., 2009; Stylianopoulos et al., 2010, and possibly on cells—is introduced in the presently disclosed subject matter by the chemical moiety dimethyl ammonium propane (DAP). Contrary to previously reported DAP-containing liposomes, the titratable charge was designed to be located at the free end of the PEG-chains forming the liposome corona and to not be conjugated directly on the lipid headgroups (FIG. 1, the molecule's structure). This surface architecture was hypothesized (and is demonstrated herein) to promote electrostatic adhesion/adsorption on extracellular compartments within the tumor and to minimize internalization by cells. The latter is critical in the present strategy so that while delaying the nanocarrier clearance from the tumor to also increase the fraction of released drug in the tumor interstitium. The particular moiety was chosen because the intrinsic pKa of the free DAP (between 6.58 and 6.81), Bailey et al., 1994, is reported to be comparable to observed pH values in the tumor acidic interstitium, Helmlinger et al., 1997; Vaupel et al., 1989, enabling, therefore, selective adhering properties to lipid-based nanocarriers.

The presently disclosed subject matter, in part, characterizes the extent of the pH-dependent drug release property and pH-dependent adhesion property of lipid-based nanocarriers, and their role on affecting the transport of nanocarriers and their contents in 3D multicellular spheroids. The effect of the adhesion property on the biodistributions of lipid-based nanocarriers is evaluated and, finally, the significance of each property and their combination on controlling the growth of spheroids and of spontaneous TNBC metastases in vivo is evaluated.

### 1.2 Materials and Methods

### 1.2.1 Materials

All lipid products were obtained from Avanti Polar lipids (Alabaster, USA) including 1,2-distearoyl-sn-glycero-3-phosphocoline (DSPC), 1,2-diarachidoyl-sn-glycero-3-phosphocholine (20PC), 1,2-dioctadecanoyl-sn-glycero-3-phospho-L-serine (sodium salt) (DSPS), 1,2-distearoyl-sn-glycero-3-phosphoethanolamine-N-[amino(polyethylene glycol)-2000](ammonium salt) (DSPE-PEG(2000)), and 1,2-dipalmitoyl-sn-glycero-3-phosphoethanolamine-N-(Lissamine Rhodamine B Sulfonyl) (Ammonium Salt) (DPPE-Rhodamine). The functionalized lipid 1,2-distearoyl-sn-glycero-3-phosphoethanolamine-N-PEG2000-dimenthylammonium propanoyl (DSPE-PEG(2000)-DAP) was custom synthesized by Avanti Polar lipids. All materials are described in detail herein below.

### 1.2.2 Cell Lines

MDA-MB-231 and MDA-MB-436 hTNBC cell lines were obtained from the American Type Culture Collection (ATCC, Rockville, MC, USA) and were cultured in DMEM Media and in RPMI 1640, respectively, both supplemented with 10% fetal bovine serum and 1% penicillin streptomycin 100× solution at 37° C. with 5% CO₂. Mouse-derived MDA-MB-231 sublines were developed from primary and metastatic tumors formed in NOD scid gamma (NSG) female mice (vide infra) as described before. Iorns, 2012. Briefly, tumors were removed, ground, plated in petri dishes with DMEM media, and were grown for several weeks when only MDA-MB-231 subline cells survived which were then propagated and frozen. The following sublines (Table 1-1) were developed and studied: MDA-MB-231-PRI3 was derived from the (primary) orthotopic MDA-MB-231 xenograft tumor (PM) of mouse numbered 3, the MDA-MB-231-ALN2 was derived from an axillary lymph node (ALN) metastasis from mouse numbered 2, and MDA-MB-231-LUNG1 was derived from lung (LUNG) surface metastases of mouse numbered 1. These metastatic sites were chosen due to their frequency in appearance: both ALN and lung metastases were detected in 45 out of 45 mice (100%).

TABLE 1-1
Cell line characterization.

| | | | | IC₅₀ of free cisplatin | |
|---|---|---|---|---|---|
| | | Metastatic | Doubling Time | (μM)* | |
| Subline | Mouse # | Site | (hrs) | pH 7.4 | pH 6.5 |
| MDA-MB-231 | 3 | Primary Tumor | 26.1 ± 1.7 | 32.4 ± 7.8 | 37.7 ± 13.4 |
| (PRI3) | | | | | |
| MDA-MB-231 | 1 | Lung | 25.1 ± 0.4 | 40.2 ± 6.4 | 42.6 ± 6.2 |
| (LUNG1) | | | | | |
| MDA-MB-231 | 2 | Axillary Lymph | 26.5 ± 0.2 | 39.2 ± 5.9 | 40.2 ± 7.4 |
| (ALN2) | | Node | | | |
| MDA-MB-231 | N/A | N/A | 34.8 ± 1.3 | 45.2 ± 8.2 | 47.7 ± 4.4 |
| (ATCC) | | | | | |
| MDA-MB-436 | N/A | N/A | 41.0 ± 1.9 | 2.46 ± 0.90 | 3.2 ± 0.73 |
| (ATCC) | | | | | |

### 1.2.3 Liposome Preparation and Loading of Cisplatin

The compositions of all lipid nanoparticles studied are listed on Table 1-2 presented herein below. All liposomes were PEGylated with 8 mole % DSPE-PEG(2000) lipid. The pH-triggered releasing property on lipid bilayers was introduced by combining a zwitterionic lipid (phosphatidyl choline) with a titratable anionic lipid (phosphatidyl serine) with four carbons difference in the lengths of their corresponding saturated acyl tails (20:0-PC and 16:0-PS, DPPS, respectively) (compositions numbered 3 and 5). The pH-triggered adhesion property on lipid nanoparticles was introduced by replacing the PEGylated lipid, DSPE-PEG(2000), with the ‘adhesion lipid’ DSPE-PEG(2000)-DAP (compositions numbered 2, 3 and 4). For the studies aiming to demonstrate the liposome properties bearing the ‘adhesion lipid’, DSPE-PEG(2000)-DAP, comparison was made to liposomes with ‘surface charge’, i.e., with the identical titratable (cationic) moiety DAP conjugated directly on the lipid headgroups, DSPE-DAP (composition numbered 1). All compositions were labeled with 0.125 mole % DPPE-Rhodamine lipid. Lipid-based nanocarriers were prepared using the thin film hydration method. Briefly, the dry lipid film (10 to 80 μmoles total lipid) was hydrated with 1 mL of PBS (10 mM phosphate buffered saline with 1 mM EDTA) at pH 7.4, and this suspension was then annealed at 60° C. for 2 hours following extrusion for 21 times through two stacked 100-nm pore-sized polycarbonate membranes at 80° C. Cisplatin was then passively loaded into liposomes which were first passed through a Sepharose 4B column. Stras et al., 2013.

TABLE 1-2
Compositions of Liposomes (in mole ratios). Lipid nanocarrier compositions studied included a ‘releasing’
and a ‘non-releasing’ liposome structure containing 20PC:DPPS:Cholesterol:DSPE-PEG at 0.51:0.33:0.08:0.08
mole ratios and DSPC:DSPS:Cholesterol:DSPE-PEG at 0.56:0.24:0.12:0.08 mole ratios, respectively,
as reported before. Stras et al., 2016. Both liposome types were functionalized with 8 mole % DSPE-
PEG(2000)-DAP replacing the non-functionalized DSPE-PEG(2000) lipid.

| Liposomes properties [symbol] | | | | | | | | | | |
|---|---|---|---|---|---|---|---|---|---|---|
| Composition | Release | Adhesion | | 18PS | 18PC | 18PS | DSPE-PEG-DAP | DSPE-DAP | | |
| number | (R) | (A) [symbol] | 20PC | (DPPS) | (DSPC) | (DSPS) | (adhesion lipid) | ( [symbol] DAP) | DSPE-PEG | cholesterol |
| | Liposome compositions shown below do not contain negatively charged lipids* | | | | | | | | | |
| 1 | N/A | Surface charge** | 0. [symbol] 5 | | | | | 0.04 | 0.002 | |
| 2 | N/A | + | 0.73 | | | | 0.08 | | | 0.18 |
| | Liposome compositions shown below contain the same amount of the negatively charged, [symbol] lipid, [symbol] ** | | | | | | | | | |
| 3 | + | + | 0.51 | 0.24 | | | 0.08 | | | 0 08 |
| 4 | − | + | | | 0.56 | 0.24 | 0.08 | | | 0.08 |
| 5 | + | − | 0.51 | 0.24 | | | | | 0.08 | 0.08 |
| 6 | − | − | | | 0.56 | 0.24 | | | 0.08 | 0.08 |

[symbol]

indicates data missing or illegible when filed

After loading, unencapsulated cisplatin was removed from liposomes using a 10-cm Sephadex G50 column, eluted with PBS at pH 7.4. An aliquot of the collected liposome fraction was lysed with 0.5% Triton-X 100, and the platinum content was measured using a Graphite Furnace Atomic Absorption Spectrophotometer (GFAAS) (Buck Scientific, using a Hollow Cathode Pt 365.9-nm lamp) and quantified by comparison to a calibration curve as reported before. Stras et al., 2016. The lipid content was evaluated using the Stewart's Assay. Stewart, 1980. Before incubation with cells, all lipid nanocarriers were sterilized by filtration (200-μm filters, VWR, Radnor, Pa.).

### 1.2.4 Liposome Characterization

The size and zeta potential of lipid nanocarriers were determined using a Zetasizer Nano ZS 90 (Malvern, United Kingdom). Samples were diluted in PBS (10 mM phosphate buffer, 150 mM NaCl, 300 mOsm) or low salt PBS (10 mM phosphate buffer, 15 mM NaCl, 280 mM sucrose and 300 mOsm), respectively. Retention of cisplatin by nanocarriers was performed in 10% FBS-supplemented cell culture media in the presence of cells, for a 6-hour incubation period. At the end of incubation, the liposome suspension (upon separation of cells) was run through a Sephadex G50 column to remove released cisplatin from liposomes, and the content of platinum in the collected fractions was quantified using the GFAAS.

### 1.2.5 Binding and Imaging Studies of Lipid-Based Nanocarriers to Cells

For cell binding measurements, lipid vesicles labeled with 1.6 mole % DPPE-rhodamine lipid were incubated with cells in suspension at the ratio of 10⁶ liposomes per cell (0.2 mM total lipid and 0.8×10⁶ cells per mL). Aliquots were placed on ice (at 4° C.) or in a humidified incubator at 37° C. and 5% CO₂ for three hours to allow lipid vesicles to bind and/or become internalized by cells in suspension. Vesicles associated by cells were isolated by centrifugation, were resuspended in 750 μL of DI water, were sonicated for 10 minutes and finally were then mixed with acidified Isopropanol (10% HCl, 90% IPA) at 1:1: v/v ratio to ensure complete cell lysis before measurement of rhodamine's fluorescence intensity (ex/em: 550 nm/590 nm) using a spectrofluorometer (Fluorolog FL-1039/40, Horiba, Edison, N.J.).

To evaluate the extent of reversible association of lipid vesicles with cells, after the initial incubation for three hours in pH 6.5, aliquots of the cell-lipid vesicle suspensions were further incubated at pH 7.4 for an hour in fresh media. Upon completion of incubation, vesicles still associated with cells were measured as stated above.

For imaging, MDA-MB-231 cells were harvested with 0.05% Trypsin (w/w), and 100,000 cells were plated in 35 mm glass bottom dishes to adhere overnight. Cell monolayers were then incubated with 1.6 mole % DPPE-Rhodamine-labeled lipid vesicles at a ratio of 10⁶ liposomes per cell. After completion of incubation for 3 hours in media (DMEM) adjusted to pH 7.4 and 6.5, the cells were washed twice with PBS prior to Hoechst 33342 staining, and again washed twice with PBS. Z-stack images (step size=1 μm) of the cell monolayers were imaged using a Nikon A1 confocal laser scanning microscope (RFP for Rhodamine:excitation/emission wavelengths 561/595 nm; DAPI for Hoescht:excitation/emission wavelengths 405/450 nm) under an oil immersion 60× objective.

### 1.2.6 Decellularization of Tumors

Tumors were harvested from mice bearing orthotopic MDA-MB-231 xenografts and were placed into tubes with 10 ml of 1% sodium dodecyl sulfate (SDS) dissolved in deionized water and supplemented with 1% Pen-Strep. This treatment has been shown to remove all cells, while leaving extracellular matrix (ECM) proteins fully intact, creating a decellularized ECM scaffold. Ott et al., 2008.

The tubes were rotated until complete decellularization was achieved, with the SDS solution replaced every 24 hours. After approximately 3 to 4 days, depending on tumor size, complete decellularization was reached, marked by tumors turning completely white and translucent. The tumor ECM scaffolds were then washed several times with cold PBS to ensure the removal of SDS. Complete tumor decellularization was verified by imaging Hoechst stained tumor slices (30-μm thickness) before/after treatment to confirm no cells remained associated with the tumor ECM. Lü et al., 2014.

Finally, the decellularized tumors were sliced into small pieces (about 10 mm³) and skewered on to stainless steel pins for imaging.

1.2.7 Clearance Profiles of Lipid-Based Nanocarriers from Decellularized Tumor ECM

Tumor ECM scaffolds on-a-pin (as described above) were each placed into a 150-mm×25-mm petri dish with 200 mL of fresh PBS at pH 6.5. Once the tumor/pin was placed into the dish, the dish was not moved and fluorescent images were obtained every 10 minutes for 16 hours using an Olympus IX81 inverted fluorescence microscope with an 10× objective. The same section of the same tumor piece was measured for lipid vesicles with and without the titratable moiety DAP to account for any heterogeneities in the tumor sections. To analyze, the average intensity of the same region of a tumor piece was measured at each time point in ImageJ. The intensities were then normalized by the initial average intensity, and a single exponential decay curve was fit to the data. Using this fitting, the areas under the curve and the half-life were calculated for the different liposome compositions on the same tumor piece and compared.

### 1.2.8 Evaluation of IC₅₀ Values on Cell Monolayers

To determine the IC₅₀ of liposomal cisplatin and of free cisplatin, 20,000 cells per well were plated in 96-well plates. A range of concentrations of sterilized liposomes (containing or not cisplatin) or of free cisplatin was added to the wells mixed in media (with 10% FBS) at pH 7.4 and pH 6.5. Upon completion of a 6-hour incubation, cells were washed twice with PBS, and 10% FBS-supplemented fresh media was then added to wells. After two doubling times an MTT assay (Promega, Madison, Wis.) was used (following the vendor's instructions) to evaluate percent cell viability. Absorbance was read at 570 nm.

### 1.2.9 Multicellular Spheroid Formation and Characterization

Formation of MDA-MB-231 spheroids was described previously. Stras et al., 2016. To form spheroids using MDA-MB-231 mouse-derived sublines or the MDA-MB-436 cell line, cells were trypsinized and diluted in DMEM or RPMI 1640, respectively, with 2.5% (v/v) Matrigel™. Cells were plated at a density of 150-175 cells per well in polyHEMA coated U-shaped 96-well plates. Media, plates, and materials were kept at 4° C. to prevent gelation of Matrigel™. Plates were then centrifuged at 1000×g for 3 minutes to pellet cells, and after 10-11 days spheroids reached the desired size of 400 μm in diameter.

To determine the interstitial pH gradients, spheroids were incubated with SNARF-4F, a membrane impermeant pH indicator (ex: 488 nm, em: 580 nm and 640 nm) as described before,¹³ and also described in detail herein below.

1.2.10 Spatiotemporal Profiles of Liposomes and their Contents in Spheroids

To determine the uptake and clearance of liposomes and their contents in spheroids, liposomes were prepared with 1 mole % DPPE-Rhodamine lipid and were loaded with CFDA-SE (ex/em: 497 nm/517 nm); final CFDA-SE concentration in liposome suspension was 800 nM. Averaged CFDA:Lipid mole ratios for all constructs were similar; 4.26×10⁻⁵±0.623×10⁻⁵: 1 (n=4).

Spheroids were incubated for 6 hours with liposomes labeled with DPPE-rhodamine and encapsulating CFDA-SE (ex/em: 497 nm/517 nm; used as a fluorescent surrogate for cisplatin) at 1 mM total lipid and 40 nM CFDA-SE, and upon completion of incubation spheroids were transferred to fresh media. At different time points (3 and 6 hours during incubation with liposomes; and 0.5, 1, 2, 4, and 24 hours following completion of incubation) several spheroids were sampled in a volume of about 1 μL and frozen in Cryochrome gel at −80° C. Spheroids without any treatment were frozen to be used as background. The samples were then sliced on a cryotome at 20-μm thickness and the equatorial slices were imaged on an Olympus IX80 fluorescence microscope. To evaluate the radial fluorescence intensities in spheroid slices, images were analyzed using an in-house developed eroding code in Matlab averaging the intensity of each 5 μm-wide concentric ring of the spheroid as described before. Zhu et al., 2017. Calibration curves were evaluated using the same microscope on known concentrations of rhodamine-labeled liposomes and of CFDA-SE imaged in a quartz cuvette of optical pathlength identical to the thickness of the spheroid slices (20 μm). The spatial distributions were integrated over time (using the trapezoid rule) to express the time-integrated lipid concentrations or CFDA radial concentrations for each construct within spheroids.

### 1.2.11 Spheroid Treatment

Spheroids were incubated with different forms of cisplatin (liposomal or free) for 6 hours, washed once, and then moved to wells of fresh media. The % change in volume of spheroids over time (Vₜ/Vₒ×100) was monitored till the non-treated spheroids reached a plateau in growth. At that point the spheroids were plated on adherent cell culture 96-well plates (one spheroid per well) and were allowed to grow. When the control spheroids reached confluency, cells were trypsizined and counted using a Z1 Coulter Counter (Indianapolis, Ind.). The number of live cells was reported as % outgrowth relative to the counted numbers of non-treated cells.

### 1.2.12 Animal Studies

NSG female Mice (4-5 weeks old) were purchased from Johns Hopkins University Breeding Facility, and studies were performed per Institutional Animal Care and Use Committee protocol (IACUC). For orthotopic tumor inoculation, a small incision was made on each mouse, allowing injection of 0.5 million MDA-MB-231 cells suspended in 100 μL of serum-free cell culture media into the second mammary fat pad on the right side of animals.

For liposome biodistributions, loaded liposomes were prepared as described before, Karve et al., 2009, and were injected intravenously in animals at doses of 7-12 μCi. The exact injected activity into individual animals was determined by measuring each of the filled syringes in a dose calibrator and subtracting the residual activity post injection. At different time points, animals were sacrificed, blood was collected through a ventricle heart puncture, and tumors/organs of concern were harvested, weighted and their associated radioactivity was measured in a gamma counter (Packard Cobra II Auto-Gamma, Model E5003).

The metastatic animal model studied herein, Iorns et al., 2012, was found to be especially aggressive resulting in fast growth of tumor burden—mainly due to growth of the orthotopic tumor—demanding animal sacrifice only a few days after detection of metastatic sites by imaging (usually 16±4 days after tumor inoculation). To allow for more time to potentially study the effect on the growth control of metastases of the different therapeutic modalities, the orthotopic xenografts were completely resected, and the growth of metastases was followed over time. This approach, of surgically removing the orthotopic tumor, prolonged the life expectancy of animals (up to 2-3 weeks, in the absence of treatment), and also better emulated the current clinical practice.

In particular, for efficacy studies on controlling the growth of metastatic tumors, orthotopic tumors were removed surgically when they reached 160-200 mm³. When formation of metastatic tumors in the axillary lymph nodes (ALN) were confirmed by MRI (approximately 2.5 weeks after surgery), mice were treated with different types of liposomal cisplatin and with free cisplatin at the same dose of 7.5 mg/kg of cisplatin which was injected intravenously. Treatment groups consisted of 5-7 animals, and injections were performed three times in five-day intervals. The diameters of metastatic tumors at the start of therapy ranged from 0.5 mm to 2.0 mm. Metastatic tumor growth was monitored by MRI once a week over the course of the experiment, and on the day animals were euthanized. To determine change in volume of metastases, MRI images were analyzed using Vivoquant software (Invicro LLC, Boston, Mass.). Per IACUC protocol, animals were euthanized if they met conditions for euthanasia.

### 1.2.13 Statistical Analysis

Results are reported as the arithmetic mean of n independent measurements±the standard deviation. Student's unpaired t test was used to calculate significant differences in killing efficacy between the various constructs. p-values less than 0.05 were considered to be significant.

### 1.3 Representative Results

### 1.3.1 Characterization of DSPE-PEG(2000)-DAP Lipid (the Adhesion Lipid)

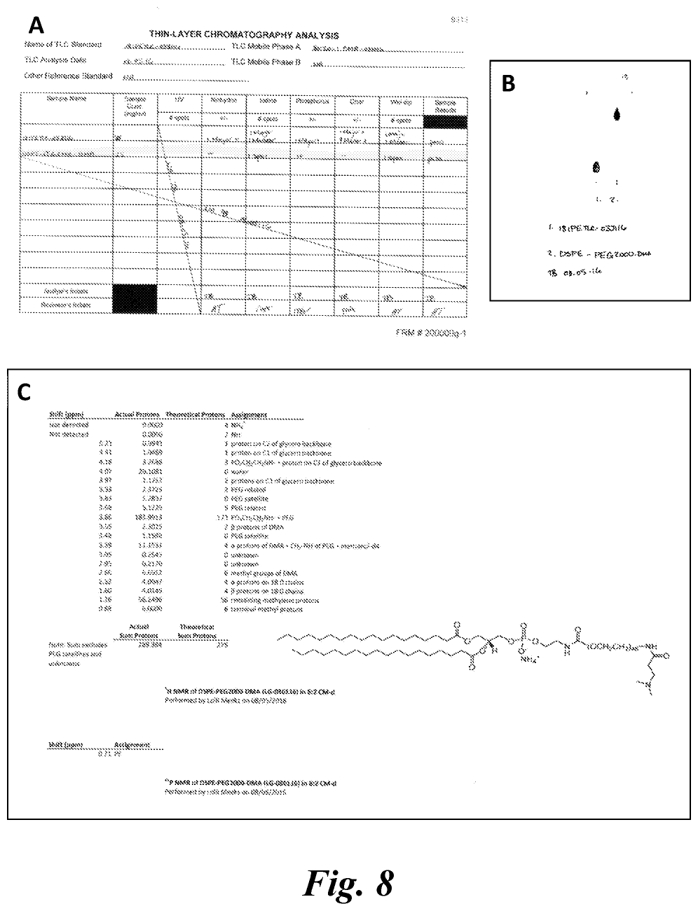

The purity and molecular weight of the functionalized lipid DSPE-PEG(2000)-DAP were >99% and 2889.62 g/mol, respectively, as reported by Avanti Polar Lipids (details in FIG. 8A, FIG. 8B, and FIG. 8C).

The rows numbered 1 and 2 of Table 1-3 show that on liposomes composed only of zwitterionic lipid headgroups (phosphatidyl choline) addition of the titratable group DAP as DSPE-DAP (surface charge) and DSPE-PEG(2000)-DAP (charge on the free ends of PEG-chains, the adhesion lipid), respectively, resulted in more positive zeta potential values with lowering pH. The increase in zeta potential's value was attributed to the protonation of the DAP moiety (the pKa of DAP is reported to be between 6.58 and 6.81).²⁴ Notably, the value of zeta potential alone was not an adequate property to characterize the extent of protonation (and different location) of cationic charge on liposomes that in addition to the (same amount of) DSPE-PEG(2000)-DAP lipid (as in row numbered 2) contained also the anionic titratable lipid headgroups (phosphatidyl serine) (shown in rows numbered 3 and 4). Instead, the changes in zeta potential on these liposomes were identified to be more relevant demonstrating less negative values of a ‘collective’ zeta potential with lowering pH. The measured zeta potential values were interpreted to indicate two protonation processes: first, the protonation of the anionic phosphatidyl serine (with apparent pKa of about 6.5), Bajagur Kempegowda et al., 2009, resulting in neutral moieties on the lipid headgroups and the protonation of DSPE-PEG(2000)-DAP resulting in cationic charges on the free ends of PEG-chains.

### 1.3.2 Liposome Characterization

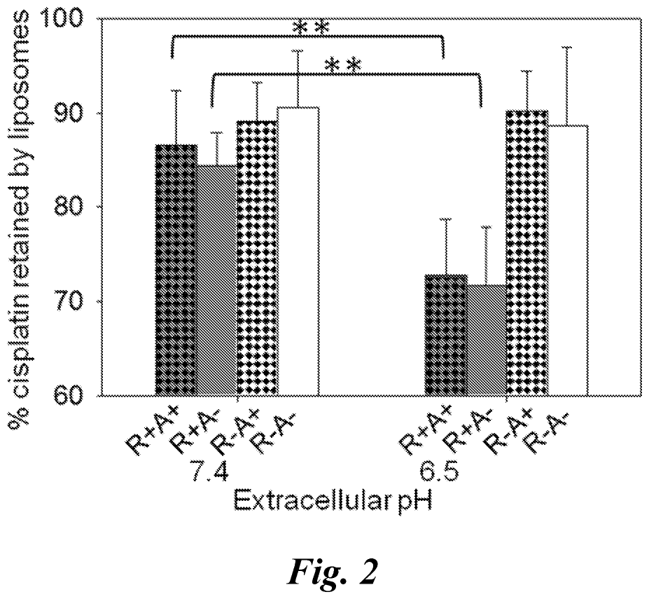

Liposomes, regardless of composition, had similar sizes (ranging from 109 to 121 nm), loading efficiencies and Drug-to-Lipid Ratios (Table 1-3). The pH-releasing liposomes (FIG. 2) exhibited significant release (approximately 15%) of encapsulated cisplatin at pH 6.5 relative to pH 7.4 (p-values<0.01) independent of the presence or absence of DAP-functionalization. As expected by design, non-releasing liposomes stably retained the encapsulated cisplatin which was not affected by the pH acidification.

1.3.3 Liposome Interactions with Cells and the Tumor ECM: Role of the Location of the Titratable Cationic Moiety

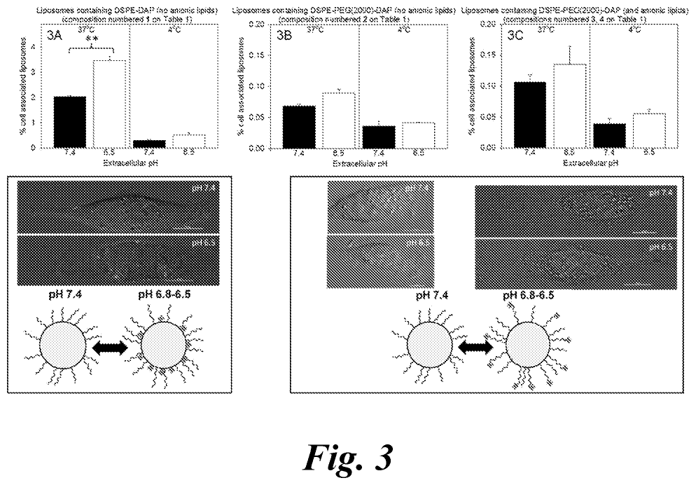

Liposomes functionalized with the DAP moiety directly attached on lipid headgroups using DSPE-DAP lipids (surface charge), indicated by composition 1 on Tables 2 and 3, exhibited measurable changes in their association with cells with lowering pH from 7.4 to 6.5 as shown on FIG. 3A. Greater extents of internalization were observed at the acidic extracellular pH value at which the lipid-surface DAP group was protonated. As hypothesized, moving the location of the titratable DAP group from the lipid headgroups (DSPE-DAP) to the free ends of PEG-chains which were grafted on liposomes (DSPE-PEG(2000)-DAP), resulted in a more than one order of magnitude decrease on the extent of liposomes which were adhered to and/or internalized by cells (FIG. 3B and FIG. 3C).

Confocal cell imaging confirmed that the location of cationic charge on liposomes may enhance (surface charge) or reduce (charge on end of PEG-chains) the extents of internalization by cells of DAP-containing liposomes, and additionally demonstrated pronounced differences of the type of interactions with cell membranes. Liposomes with protonated DAP directly conjugated on lipid headgroups (FIG. 3A, pH 6.5) exhibited localization at the cell plasma membranes, which was contrary to the lack of such subcellular localization of liposomes when the latter were functionalized with DAP on the free ends of grafted PEG-chains. Both types of lipid nanocarriers exhibited some type of endocytosis suggested by the observed punctuate fluorescence.

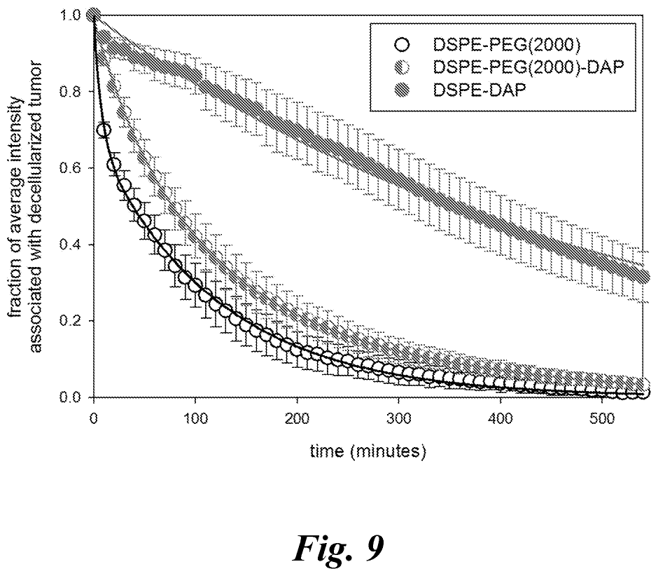

Supporting, of the goals of this study for intratumoral adhesion with low cell internalization, was the finding that localization of cationic DAP on the free ends of PEG-chains enhanced the liposome interactions with the extracellular matrix. In particular, upon incubation with the decellularized extracellular matrix derived from MDA-MB-231 tumors from mice, liposomes functionalized with DSPE-PEG(2000)-DAP exhibited slower clearance kinetics (half-life of clearance, τ½=99±17 minutes) compared to liposomes without the DAP-moiety (half-life of clearance, τ½=79±24 minutes) (p-value=0.08 by paired-t test). Clearance kinetics were fitted by a single exponential function (FIG. 9). The above characterization of liposome-cell interactions supported the term ‘adhesion lipid’ for DSPE-PEG(2000)-DAP.

### 1.3.4 Cell Monolayers: Effect of the Adhesion and Release Properties of Liposomal Cisplatin on IC₅₀ Values

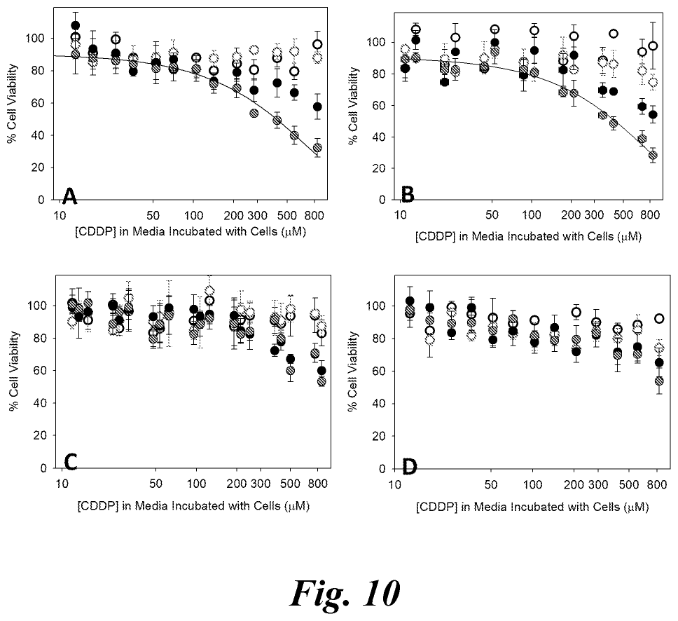

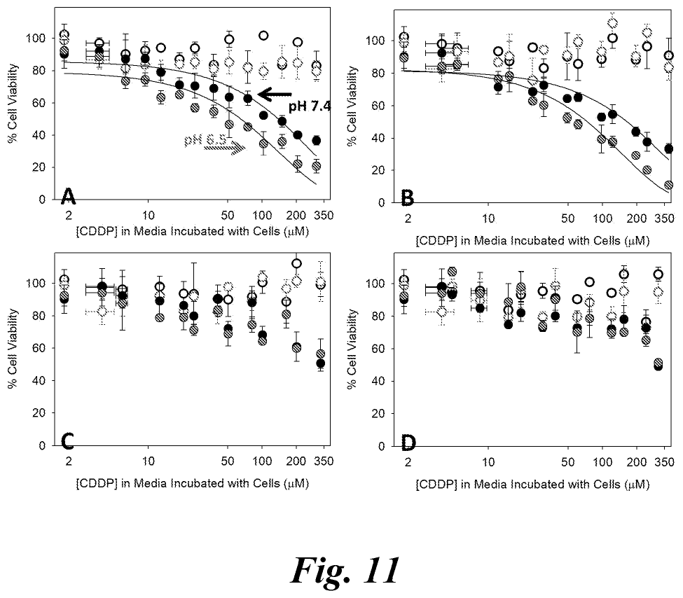

Table 1-4 shows that pH-releasing liposomes loaded with cisplatin exhibited IC₅₀ values, which were significantly lower for the acidic pH conditions. Non-releasing liposomes did not result in measurable IC₅₀ values at the conditions studied (see also FIG. 10 and FIG. 11). The killing efficacy of pH-releasing liposomes, as was shown before, is thought to be driven by the extracellularly released cisplatin, which then diffuses across the plasma membrane. Stras et al., 2016. The IC₅₀ values of free cisplatin were independent of the extracellular pH (Table 1-1).

Given the absence of significant cell internalization of liposomes functionalized with the adhesion lipid (DSPE-PEG(2000)-DAP) (FIG. 3), it was not unexpected that the IC₅₀ values of releasing liposomes did not depend on the presence or absence of PEG(2000)-DAP functionalization (Table 1-4). Liposomes not containing cisplatin did not result in significant cell kill (FIG. 10 and FIG. 11).

The TNBC cell line MDA-MB-436 was more sensitive to the platinum compound than the MDA-MB-231 line, in agreement with previous reports. Stefansson et al., 2012. The MDA-MB-436 is a BRCA-1 mutated TNBC cell line exhibiting aberrant DNA double-strand break repair mechanisms which to some extent have been the basis for increased clinical use of platinum-derived agents, Telli, 2014, against TNBC. Dawood et al., 2010; Anders et al., 2013. Interestingly, the MDA-MB-231 cell line and its animal derived sub-lines exhibited comparable drug sensitivities (not statistically different, Table 1-1) so the subsequent evaluation in spheroids of liposomal cisplatin forms was performed on the MDA-MB-231 (and on MDA-MB-436) as obtained from ATCC. 1.3.5 Multicellular spheroids: effect of the adhesion and release properties on microdistribution heterogeneities and efficacy

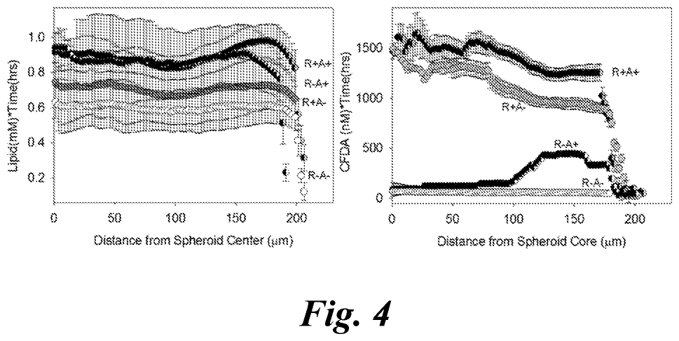

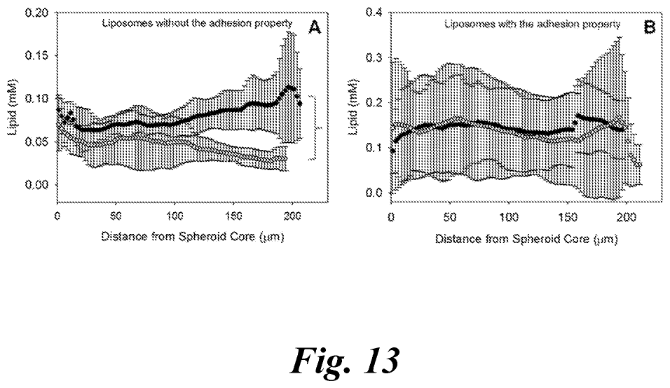

The adhesion property (via inclusion of the DSPE-PEG(2000)-DAP in liposomes) increased significantly the time-integrated lipid concentrations (FIG. 4A) within spheroids partly due to delayed liposome clearance from spheroids (see FIG. 13). Importantly, the time-integrated concentrations of the cisplatin surrogate (both encapsulated by liposomes and released, FIG. 4B) demonstrated higher values and more uniform distributions in the following order: liposomes with release and adhesion>liposomes with release without adhesion>liposomes without release with adhesion>liposomes without release and without adhesion.

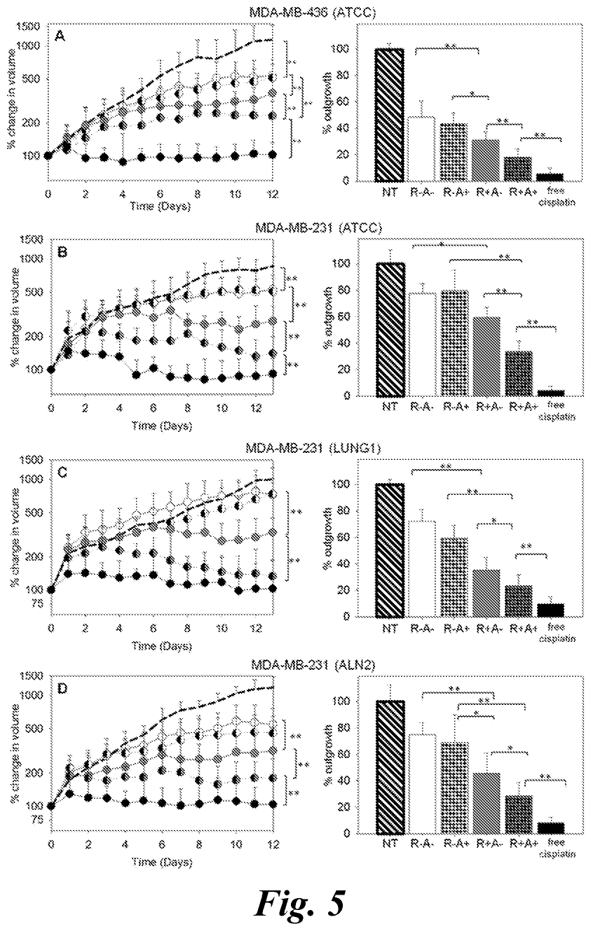

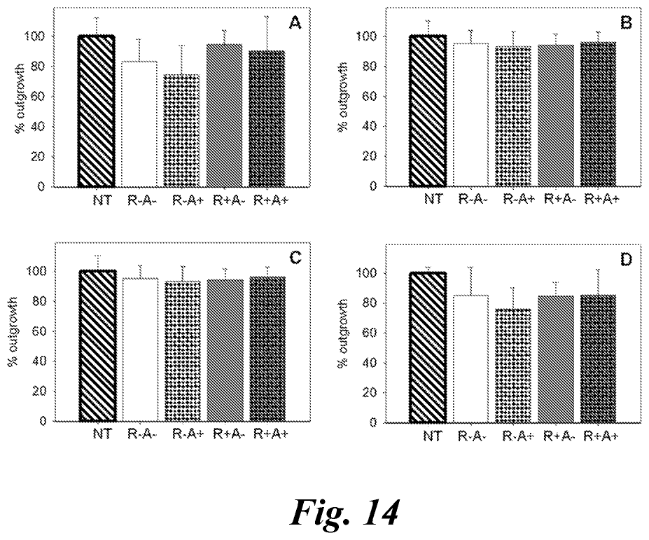

The efficacy of liposomal cisplatin in controlling the growth and outgrowth of TNBC spheroids was strongly correlated with the time-integrated microdistributions of cisplatin surrogates from FIG. 4B. Accordingly, on spheroids formed by MDA-MB-231 and MDA-MB-436 cell lines (FIG. 5A, FIG. 5B, FIG. 5C, and FIG. 5D), the efficacy of liposomal cisplatin properties was identical to the exact same order of combinations of the release and adhesion properties shown above in terms of the drug microdistributions from FIG. 4B. Liposomes not containing cisplatin did not affect spheroid growth and/or outgrowth (FIG. 14).

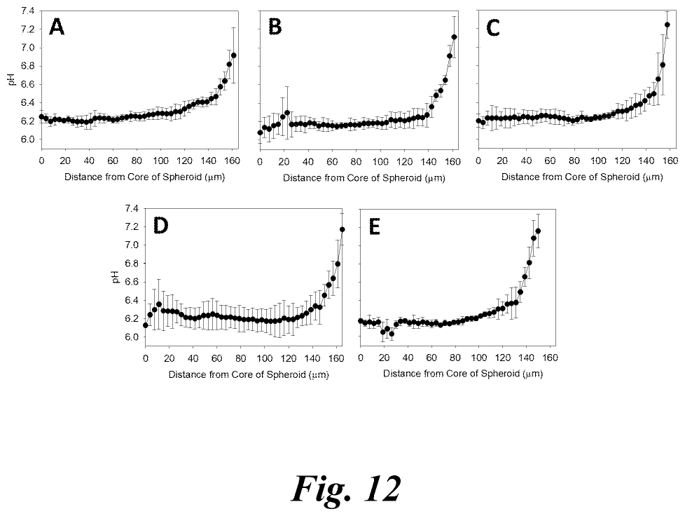

Both the adhesion and release properties were activated by the slightly acidic pH in the spheroid interstitium. The latter was confirmed for all five cell lines with pH gradient values decreasing from the spheroid edge (pH about 7.2 to about 7.0) toward the core where the average pH value was 6.2±0.1 (FIG. 12A, FIG. 12B, FIG. 12C, FIG. 12D, and FIG. 12E).

### 1.3.6 Liposome Biodistribution: Effect of the Adhesion Property

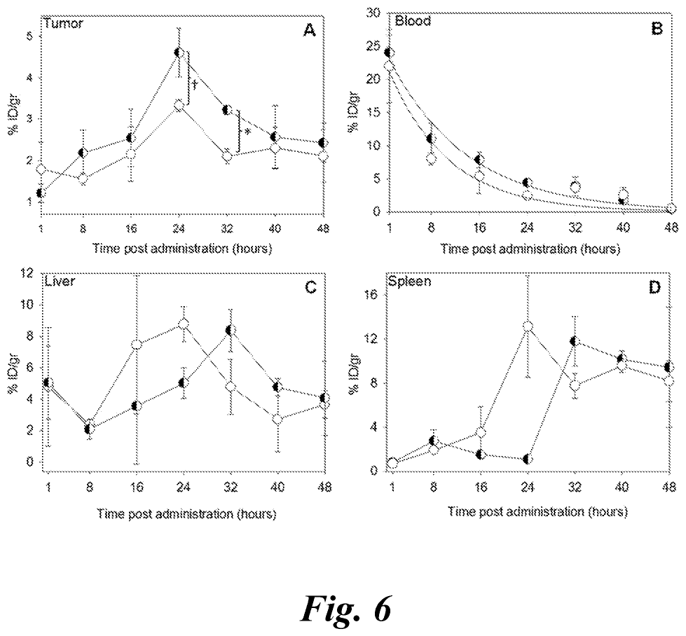

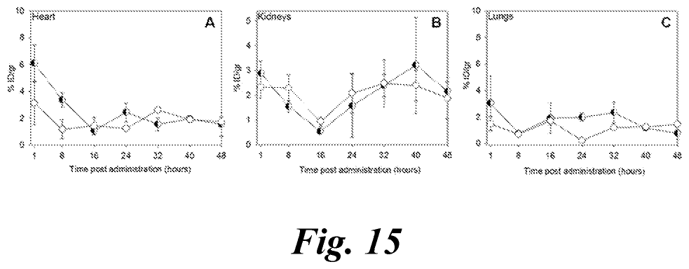

FIG. 6A shows that i.v. administered adhering liposomes (containing DSPE-PEG(2000)-DAP, compositions designated R+ in Table 1-2 and Table 1-3) exhibited higher tumor uptake and slower clearance from tumors (greater AUCₜᵤₘₒᵣ) compared to liposomes without the adhesion property (compositions designated R−). Importantly, the adhesion property did not significantly affect the blood clearance kinetics of liposomes (FIG. 6B) but delayed the uptake and clearance of liposomes from the liver and spleen, the two major off-target uptake sites (FIG. 6C and FIG. 6D), and did not affect the heart, lung and kidney uptake profiles (FIG. 15).

### 1.3.7 Growth Control of TNBC Metastases In Vivo

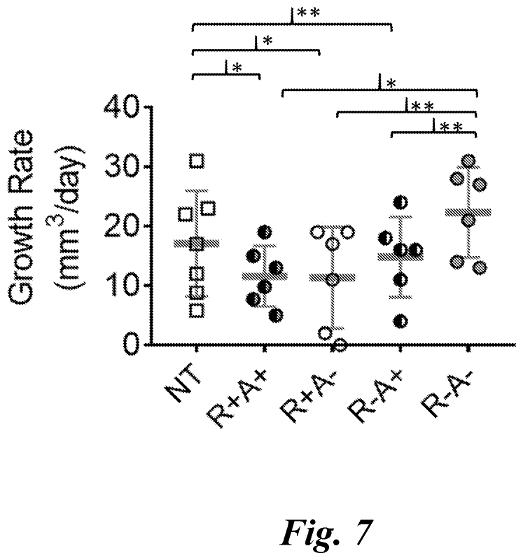

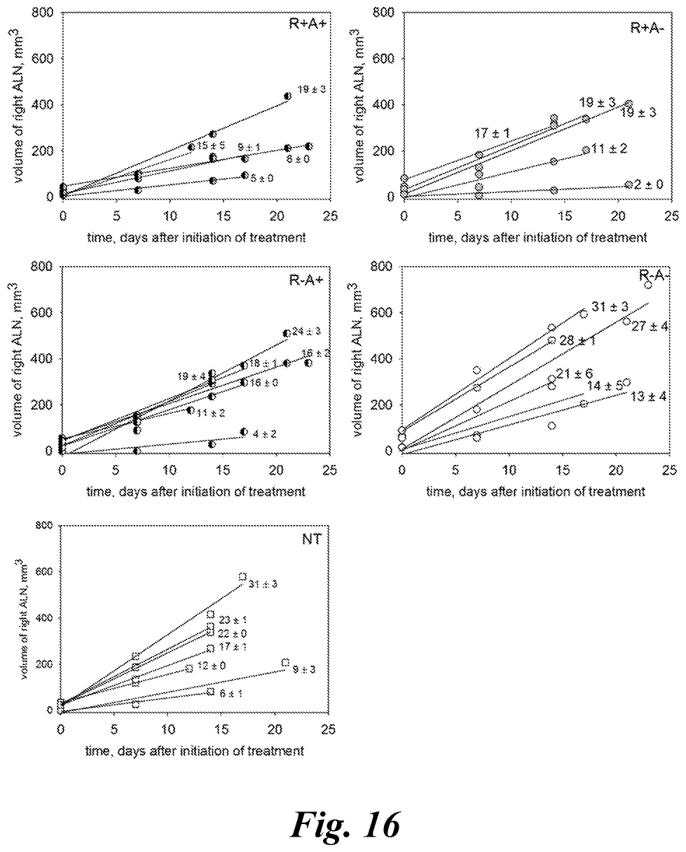

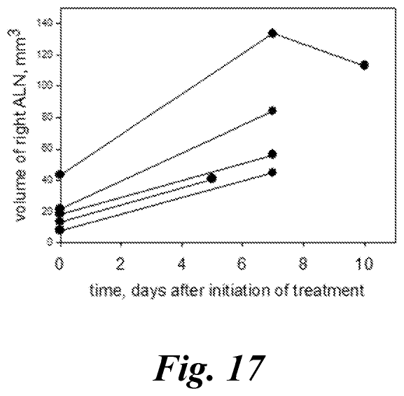

The volume growth rates (FIG. 7) of the right axillary lymph node metastases (ALN), the dominant metastases on this animal model, were significantly slower (p-values<0.05-0.01) when animals were treated with liposomal cisplatin bearing at least the release property compared to liposomal cisplatin without any of the two properties and compared to no treatment. Growth rates were calculated, to a first approximation without limiting accuracy, from the slopes of linear functions fitting the measured ALN volumes over time (FIG. 16). Additionally, the relative efficacy of property combinations in controlling the ALN tumor growth followed the same trend that was observed on growth control of spheroids which, contrary to in vivo studies, represented well-defined and well-controlled conditions. Free cisplatin, although more effective in spheroids, resulted in the shortest animal survival, possibly attributed to acute deaths (FIG. 17), for injected doses (7.5 mg/Kg) which were equal to the reported MTD. Leite et al., 2012. The end-point justification was different for animals treated with liposomal cisplatin which were euthanized mostly because of tumor ulceration or uncontrollable tumor burden.

### 1.4 Discussion

The therapeutic efficacy of delivered agents against established TNBC metastases could be improved by more uniform intratumoral drug distributions, Minchinton and Tannock, 2006; Stras et al., 2016; and Zhu et al., 2017, combined, with prolonged exposure of cancer cells to these agents. Together, these factors may cooperatively improve the drug's tumor microdistributions increasing the population of tumor cells exposed to lethal doses, therefore, potentially delaying the growth of these tumors.

To improve uniformity of drug distributions within established tumors, an approach relying on the release of the free forms of therapeutics within the tumor interstitium from nanocarriers, which were designed to not become internalized by cells was previously reported. Stras et al., 2016; Zhu et al., 2017. For therapeutic agents, such as cisplatin, which diffuse across the cell plasma membrane independent of the interstitial pH, minimization of cell internalization of the drug carriers is critical in enabling intratumoral uniformity of the distributions of therapeutic agents. Otherwise, consumption of drug carriers by cell endocytosis would decrease their population in the interstitial space and would limit the amount of free drug that would be available to penetrate into solid tumors. To effectively translate this strategy in vivo, however, the intratumoral residence times of such drug-loaded nanocarriers should be as long as possible to enable prolonged exposure of cancer cells to the agents being released.

To delay the clearance of nanocarriers from tumors without being internalized by cancer cells, lipid nanocarriers (liposomes) were functionalized with an ‘adhesion switch’ on their surface. The adhesion switch was shown to render as cationic the liposomes' PEG-corona, as opposed to the liposomes' lipid headgroups, when liposomes experienced the slightly acidic pH of the tumor interstitium. Helmlinger et al., 1997; Vaupel et al., 1989. At neutral pH—during nanocarrier circulation in the blood—the switch was shown to be mostly neutral not affecting, as demonstrated herein, the blood circulation times of liposomes. The switch resulted in adhesion of lipid nanocarriers primarily to the tumor's extracellular matrix, delaying the nanocarriers' clearance from tumors in vivo, and kept to a minimum the extent of adhesion and internalization of the nanocarriers by the cancer cells in vitro.

The location of the cationic charge was key on the design of this adhesion switch: nanocarriers functionalized with DAP on the free ends of the PEG-chains (in the form of DSPE-PEG(2000)-DAP) exhibited minimal adhesion/adsorption to cancer cells compared to PEGylated nanocarriers functionalized with DAP directly on their lipid headgroups. The steric role of PEG-chains between the lipid membrane bilayer of the liposomes and the cells' plasma membrane could have acted as the main obstacle to the close apposition of the lipid membranes, therefore, minimizing their attractive interactions. Furthermore, attempts to measure the desorption kinetics of the cell adsorbed/adhered lipid nanocarriers (when the DSPE-PEG(2000)-DAP moiety was fully protonated) resulted in findings that suggested too fast desorption rates (with half-lives shorter than 30 minutes which is the resolution of the measurement method). These relatively fast desorption kinetics also could have contributed to the limited internalization by cells whose endocytosis of nanoparticles previously reported to be of the order of 30 min. Sempkowski et al., 2016.

Liposomal encapsulation of cisplatin was previously shown to provide partial relief of the toxicities of the free agent. Leite et al., 2012; and Sempkowski et al., 2014. The presently disclosed subject matter demonstrates that in addition to controlling toxicities (relative to free cisplatin), liposomal carriers with fast interstitial drug release and/or slow tumor clearance had the potential to significantly suppress the growth rates of spontaneous TNBC metastases in vivo relative to liposomes that did not exhibit any of these two properties. The animal model used herein was found to be particularly aggressive (vide infra) to allow effects of the different property combinations on the duration of animal survival to be identified. Although the use of spheroids to capture the tumor interstitial pH gradients, transport limitations and ECM environment is by default limited, the trends in transport-response dependences that were observed in spheroids were also observed in the ability of nanocarriers with different property combinations to control the growth rate of metastases in vivo. In particular, efficacy was correlated with nanocarrier properties in the following order: release plus adhesion>release >adhesion>no release or adhesion.

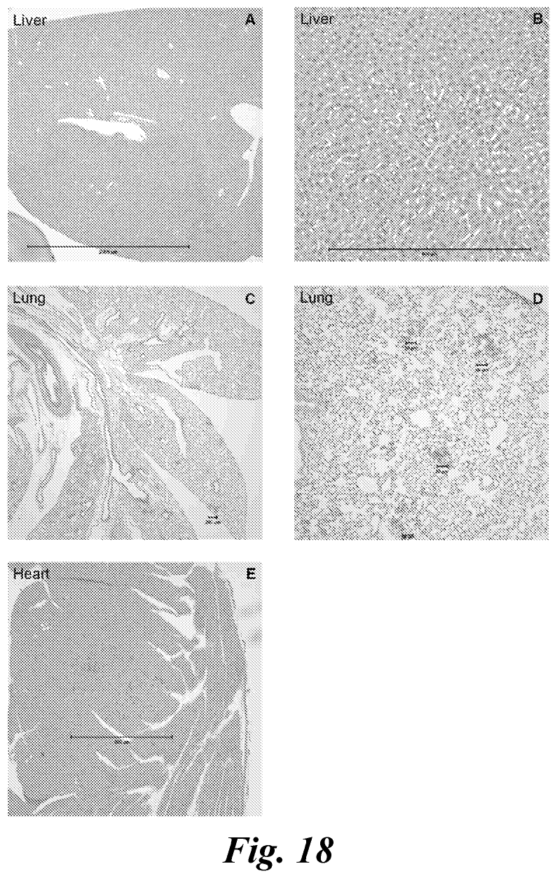

The aggressiveness of the particular animal model also was confirmed by histopathology. The main reason of death for all animals was the uncontrollable tumor burden with the exception of the acute deaths of animals, which were treated with free cisplatin. The histopathology evaluation reported that mice carried significant tumor burden especially in the liver and lungs. Tumors (or tumor emboli) were associated with necrosis in several sites and were mostly evident in the liver (FIG. 18).

To conclude, the relevance of nanoparticle-based cancer therapies to human disease has been extensively discussed and debated. Prabhakar et al., 2013. The measurable uptake of lipid nanocarriers by tumors is described by the so called EPR effect. It is well understood that not all human established metastases exhibit such behavior, but when they do exhibit uptake of nanocarriers then this uptake is strongly and favorably correlated with tumor response to nanoparticle therapies, as is clinically proven. Lee et al., 2017. An additional point that is relevant to our approach involves the acidification of the intratumoral pH (as it relates to the two key properties of nanocarriers: release and adhesion), which also is common in human TNBC, Basu et al., 2008, and has been correlated with highly aggressive tumors. Vaupel et al., 1989; Estrella et al., 2013; and Vaupel, 2004. In the future, it is envisioned that selection of patients may be conducted by personalizing nanomedicine, Wang, 2015; Shi et al., 2017, with biomarkers, in this particular case, the intratumoral microdistributions of probe-like carriers and of the tumor acidity.

### Summary

Without wishing to be bound to any one particular theory, it was thought that the control of growth of TNBC established metastases could be improved by enhancing the uniformity of drug distributions within the tumors and by prolonging the exposure of cancer cells to the delivered therapeutics. The presently disclosed subject matter demonstrates that a way to achieve this is by i.v. administration of lipid nanocarriers loaded with cisplatin exhibiting cisplatin release in the tumor interstitium while nanocarriers are designed to not become internalized by cancer cells but to, instead, adhere to the tumor ECM for enabling longer residence times within the tumors.

### 1.5 Supporting Information

### 1.5.1 Materials

In addition to lipids described hereinabove, cholesterol, cis-diamminedichloroplatinum (II) (CDDP), phosphate buffered saline (PBS) tablets, Sephodex G-50 resin, Sepharose 4B resin, and chloroform were purchased from Sigma-Aldrich Chemical (Atlanta, Ga.). Polycarbonate membranes (100-nm pore size) for extrusion, and extruder setups were purchased from Avestin (Ottawa, ON, Canada). EthylenediamineTetraacetic Acid, Disodium Salt Dihydrate (EDTA) and SNARF-4F were purchased from Thermo Fisher Scientific (Waltham, Mass.). Filters used for sterilization, with 200-micron pore diameters, were purchased from VWR (Radnor, Pa.). Media was purchased from ATCC, fetal bovine serum was purchased from Omega Scientific (Tarzana, Calif.) and penicillin streptomycin was purchased from Fisher Scientific (Waltham, Mass.). Matrigel™ used in the formation of multicellular spheroids was also purchased from Fisher Scientific.

1.5.2 Measurement of Interstitial pH Gradients (pHₑ) in Spheroids

To determine the interstitial pH gradients, spheroids, at a size of 300±30 μm, were incubated for 12 hours with SNARF-4F, a membrane impermeant pH indicator (ex: 488 nm, em: 580 nm and 640 nm) whose ratio of intensities in the red and the green channels were shown to be pH dependent.¹³ Upon completion of incubation, spheroids were washed and placed in wells of fresh media for imaging using a Zeiss LSM510 Laser Scanning Confocal Microscope. Ten micrometer thick z-stacks were obtained through the entirety of the spheroid to allow identification of the equatorial optical slice on which an in-house erosion algorithm was used to calculate the average intensities in both the green and the red channel on 5-μm concentric rings from the edge of the optical slice to the core. The fluorescent intensities of ring averaged intensities on equatorial slices of spheroids not incubated with SNARF-4F were subtracted from the above fluorescent images to correct for background intensities. A calibration curve of the ratios of the SNARF-4F intensities (acquired with the same microscope) in the red and green channels in media of known pH values was used to correlate the spheroid radial red/green average ratios to the spheroids' interstitial pH (pHₑ).

### Example 2

### Transport-Oriented Engineering of Liposomes for Delivery of A-Particle Radiotherapy: Inhibition of Solid Tumor Progression and Onset Delay of Spontaneous Metastases

### 2.1 Overview

Alpha-particle radiotherapy (αRPT) could be a strong candidate for difficult-to-treat cancers. The high killing efficacy of α-particles (that typically cause double-strand DNA breaks) is largely impervious to resistance, and their short range in tissue (5-10 cell diameters) enables localized irradiation. In solid tumors, however, the diffusion-limited penetration depths of traditional radionuclide carriers combined with the short range of α-particles result in only partial tumor irradiation compromising efficiency. To address the partial irradiation of solid tumors by the α-particle emitter Actinium-225 (²²⁵Ac), transport-oriented liposomes encapsulating ²²⁵Ac-DOTA were engineered, and their efficacy to control solid tumor growth and the subsequent onset of spontaneous metastases were evaluated in a Triple Negative Breast Cancer (TNBC) murine model.

To improve the uniformity of solid tumor irradiation, liposomes were engineered to exhibit release of the highly-diffusing ²²⁵Ac-DOTA when in the tumor interstitium and to not significantly become internalized by cancer cells. To increase the time-integrated tumor doses, liposomes were engineered to adhere to the tumors' extracellular matrix for slow tumor clearance (of the liposomes). The efficacy of ²²⁵Ac delivered by liposomes, with different combinations of the above properties (tumor interstitial release of ²²⁵Ac-DOTA and adhesion to tumor-ECM), was evaluated in vitro and in vivo.

On large MDA-MB-231 TNBC spheroids, used as surrogates of solid tumors' avascular regions, engineered liposomes combining both properties resulted in greatest inhibition of volume growth and in most significant decrease of spheroid outgrowth. On a murine model with orthotopic MDA-MB-231 TNBC xenografts that consistently develops spontaneous metastases, ²²⁵Ac-DOTA-encapsulating liposomes with both the interstitial release property and the ECM-adhesion property resulted in best inhibition of orthotopic xenograft progression and in longest delay of metastatic onset compared to liposomes lacking one or both of the transport-oriented properties. Splenic uptake was identified as the potentially dose limiting organ.

In part, the presently disclosed subject matter demonstrates the potential of this ‘transport-oriented’ approach to lead to a new class of α-particle nanoradiotherapy as a platform technology to control tumor growth and/or spreading for difficult-to-treat solid tumors.

### 2.2 Background

The tremendous clinical promise for targeted α-particle therapy against (mostly prostate) cancer, see, e.g., Kratochwil et al., 2016, has made α-particle-based therapeutics the subject of intensive research and commercial interest. This interest is reflected by the increasing number of ongoing clinical trials, including targeted therapies of Actinium-225 (²²⁵Ac). Poty et al., 2018. Even with these targeted ²²⁵Ac-approaches, however, a substantial fraction of patients will fail to respond or progress after initial response, representing an unmet clinical challenge in the development of α-particle therapy. Kratochwil et al., 2017; Sathekge et al., 2019.

Alpha-particle radiopharmaceutical therapy (αRPT) could be a strong candidate for difficult-to-treat cancers. Kratochwil et al., 2016; Song et al., 2013. The highly efficient irradiation of α-particle emitters (1-10 MeV energy), renders α-particles with a 3- to 8-fold greater relative biological effectiveness compared to photon or β-particle radiation. McDevitt et al., 2018. Alpha particles typically cause double-strand DNA breaks (dsDNA) and their high killing efficacy (1-3 tracks across the nucleus result in cell kill), see Fournier et al., 2012; Humm and Chin, 1987; Humm and Chin, 1993, also is mostly independent of the cell-oxygenation state and cell-cycle. McDevitt et al., 2018; Sofou, 2008. In addition, α-particle emitters are ideal for localized therapy due to their 40-100 μm range in tissue. In solid tumors, however, the diffusion-limited penetration depths of radionuclide carriers (such as antibodies, nanoparticles, and the like), Bhagat et al., 2012; Thurber et al., 2007; Zhu et al., 2010, combined with the short range of α-particles, Sgouros, 2008, hamper their use mostly due to partial tumor irradiation. And for αRPT the pattern of irradiation matters: areas not being hit by α-particles will likely not be killed.

Metastatic and/or recurrent Triple Negative Breast Cancer (TNBC) could benefit from αRPT. TNBC accounts for 10-20% of breast carcinomas and is (defined as being) negative in gene expression for the estrogen, progesterone, and HER2/neu receptors. Ovcaricek et al., 2011. TNBC has the lowest 5-year survival rates among all breast cancer patients. Ovcaricek et al., 2011. The poor prognosis is partly due to high proliferation and reoccurrence outside the breast, Dawood, 2010; Dent et al., 2007, combined with lack of effective therapeutic modalities. Fantini et al., 2012. Additionally, metastatic and/or recurrent TNBC may have developed chemoresistance rendering it an incurable disease. Mayer and Burstein, 2016. For such cases, key to the progression of the disease is the choice of administered therapeutic modalities which need to be tumor selective and potent against cancer cells.

To enable uniform and prolonged irradiation of solid TNBC tumors by α-particles, we designed liposomes that are tumor selective, due to their size, and, upon uptake by tumors, are engineered with two key properties: 1) to adhere on the tumors' extracellular matrix (ECM), without becoming internalized by cells, and 2) to release highly-diffusive forms of the α-particle emitters within the tumor interstitium. It was previously demonstrated that the release property enabled the released emitters to penetrate longer distances into the tumors' avascular regions compared to the emitters that were stably associated with their carriers (liposomes and/or antibodies). Zhu et al., 2017. Recently, it also was demonstrated that the ECM-adhesion property resulted in increased residence times of liposomes in the tumors, Stras et al., 2019, increasing the time-integrated tumor delivered dose. Both of the above properties were designed to be triggered by the slight acidification of the tumor interstitial pH. Helmlinger et al., 1997; Vaupel et al, 1989. This approach could be appropriate for TNBC since acidification of the tumor microenvironment (due to extracellular acidosis via enhanced glycolysis) is common on tumors of patients with TNBC, Basu et al., 2008, and is correlated with highly aggressive tumors. Vaupel et al., 1989; Estrella et al., 2013; Vaupel, 2004.

For the property of drug release in the tumor interstitium, we designed lipid nanocarriers which contained pH-responsive lipid membranes forming phase-separated lipid domains (resembling lipid patches) with lowering pH. Bandekar et al., 2012; Karve et al., 2009; Karve et al., 2008; Karve et al., 2010. During circulation in the blood, these liposomes comprise well-mixed, uniform membranes and stably retain their encapsulated contents. In the acidic tumor interstitium, occurrence of lipid-phase separation results in formation of ‘registered’ lipid patches that span the bilayer. Bandekar and Sofou, 2012. This lipid separation has been utilized to create pronounced grain boundaries around the lipid patches enabling release of the encapsulated agents which then—in a drug delivery setting—diffuse deeper into solid tumors. Zhu et al., 2017; Stras et al., 2016.

The property of nanocarrier adhesion to the tumors' ECM was mediated by a positive charge on the liposome's outer corona which is ‘turned on’ in the slightly acidic pH of the tumor interstitium. Helmlinger et al., 1997; Vaupel et al., 1989. The molecular structure of the ‘adhesion lipid’ involves the moiety dimethyl ammonium propane (DAP) conjugated on the free PEG-chain end of PEG-lipids with intrinsic pKa between approximately 6.58 and 6.81. Stras et al., 2019. It has been demonstrated, Stras et al., 2019, that liposomes with the ‘adhesion lipid’ do not significantly interact with cells (via binding and/or internalization), and the extent of their cell association is too low (an order of magnitude lower) unlike the extensive cell-interactions of previously reported cationic liposomes, Bailey and Cullis, 1997, which bear the charge directly on the surface of the lipid membrane. Sokolova et al., 2013; Lin and Alexander-Katz, 2013. Instead, liposomes with the ‘adhesion lipid’ were shown to stick on the ECM of tumors, a property which we demonstrated to play a central role in the delayed clearance of liposomes from TNBC MDA-MB-231 spheroids and from orthotopic MDA-MB-231 tumors in mice. Stras et al., 2019. In that study, delivery of cisplatin by liposomes that combined the interstitial release property and ECM-adhesion property was shown to significantly delay the growth rate of spontaneous TNBC metastases relative to traditional (non-releasing/non-ECM-adhering) liposomes in an animal model where the orthotopic xenografts were removed before administration of chemotherapy.

In the presently disclosed subject matter, on a murine model with orthotropic MDA-MB-231 TNBC xenografts that consistently develops axillary lymph node (ALN) spontaneous metastases, carriers with both the interstitial release property and the ECM-adhesion property were evaluated for the delivery of the α-particle emitter Actinium-225 (²²⁵Ac) that has a 10-day half-life and generates a total of 4 α-particles per parent decay. Sofou et al., 2004. The orthotopic xenografts were retained in this study in order to evaluate the effect of this approach to inhibit the growth of vascularized tumors and to delay the development of metastases not detectable at the initiation of therapy. Without wishing to be bound to any one particular theory, it was thought that the intratumoral acidity of the TNBC xenografts would activate both transport-oriented properties potentially enabling more uniform patterns of tumor irradiation by α-particles and resulting in better inhibition of the growth of solid tumors and/or in longer delay of the onset of spontaneous metastases. Assessment of efficacy was compared to liposomes with different combinations of the two properties.

### 2.3 Materials and Methods

### 2.3.1 Materials

All lipids including 1,2-diarachidoyl-sn-glycero-3-phosphocholine (20:0 PC), 1,2-dipalmitoyl-sn-glycero-3-phospho-L-serine (sodium salt) (DPPS), 1,2-distearoyl-sn-glycero-3-phosphoethanolamine-N-[methoxy(polyethylene glycol)-2000] (ammonium salt) (18:0 PEG2000 PE) and 1,2-dipalmitoyl-sn-glycero-3-phosphoethanolamine-N-(lissamine rhodamine B sulfonyl) (ammonium salt) (DPPE-rhodamine) were purchased from Avanti Polar Lipids (Alabaster, Ala., USA) and used without further purification (>99% purity). The ‘adhesion lipid’ 1,2-distearoyl-sn-glycero-3-phosphoethanolamine-N-PEG2000-dimenthylammonium propane/propanoyl (DSPE-PEG(2000)-DAP) was custom synthesized by Avanti Polar lipids. Stras et al., 2019. Cholesterol, Phosphate Buffered Saline (PBS), Sephadex G-50, Sepharose-4B, Chloroform, Ascorbic Acid, 4-(2-hydroxyethyl)-1-piperazineethanesulfonic acid (HEPES), Sucrose, Trisodium citrate dihydrate, Anhydrous citric acid, Poly(2-hydroxyethyl methacrylate) (polyHEMA) and calcium ionophore A23187 were purchased from Sigma-Aldrich (Atlanta, Ga., USA).

Ethylenediamine tetraacetic acid, Disodium salt dihydrate (EDTA) was purchased from Fisher Scientific (Pittsburgh, Pa., USA). Trypsin and Matrigel™ (growth factor reduced) were purchased from Corning (Corning, N.Y., USA). Penicillin-Streptomycin was purchased from ThermoFisher Scientific (Waltham, Mass., USA), and 1,4,7,10-tetraazacyclododecane-1,4,7,10-tetraacetic acid (DOTA) from Macrocyclics (Dallas, Tex., USA).

Chelex resin, chromatography and desalting columns were purchased from Bio-Rad (Hercules, Calif., USA). Syringe filters were purchased from VWR (Radnor, Pa., USA). Dulbecco's Modified Eagle Medium (DMEM) was purchased from ATCC (Manassas, Va., USA), and Fetal Bovine Serum (FBS) from Omega Scientific (Tarzana, Calif., USA). Actinium-225 (²²⁵Ac, actinium chloride) was obtained from the Oak Ridge National Laboratory.

### 2.3.2 Liposome Formation, Characterization and Radiolabeling

Liposomes (with compositions shown on Table 2-1) were formed using the thin film hydration method as previously reported. Karve et al., 2008. Briefly, the dried films were hydrated for 2 hours at 65° C. at pH 5.5 in citrate buffer (111 mM final concentration, 300 mOsm) containing 4.92 mg/mL DOTA and 11.8 mM ascorbic acid at 10 mM final lipid concentration. All solutions were prepared using distilled Chelexed water. The lipid suspension was then extruded 21 times through two stacked 100 nm pore-size polycarbonate membranes at 80° C., and liposomes were then passed through a Sepharose-4B column eluted with HEPES buffer (20 mM HEPES, pH 7.4, 300 mOsm by addition of sucrose). Size distributions and zeta potential of liposomes were measured using a NanoSeries Zetasizer (Malvern Instruments Ltd, Worcestershire, UK).

TABLE 2-1
Compositions of Liposomes (in mole ratios). Liposomes were composed of a releasing
(R+) and a non-releasing (R−) lipid membrane. Liposomes with the adhesion
property (A+) contained 8-9 mole % DSPE-PEG(2000)-DAP. Liposomes without the
adhesion property contained 9-11 mole % of the non-functionalized DSPE-PEG(2000) lipid.

| | | Lipids | | | | | |
|---|---|---|---|---|---|---|---|
| Liposome properties | | | | DSPE- | | | |
| Release | Adhesion | | | PEG(2000)- | DSPE- | | DPPE- |
| (R) | (A) | 20:0PC | DPPS | DAP | PEG(2000) | Cholesterol | rhodamine |
| − | − | 0.72 | — | — | 0.09 | 0.18 | 0.01 |
| − | + | 0.72 | — | 0.09 | — | 0.18 | 0.01 |
| + | − | 0.59 | 0.25 | — | 0.11 | 0.04 | 0.01 |
| + | + | 0.59 | 0.25 | 0.08 | 0.03 | 0.04 | 0.01 |

To radiolabel liposomes, ²²⁵AcCl₃ in 0.2 N HCl and Calcium ionophore A23187 (initially dissolved in ethanol) were mixed in Chelex-purified water at a final concentration of 8 mg/mL and were added to pre-formed liposomes. The loading was carried out for 1 hour at 76° C. at pH 7.4. To separate unentrapped ²²⁵Ac, the cooled suspension was passed through a Sephadex-G50 column eluted with PBS (1 mM EDTA, pH 7.4). The specific radioactivity of liposomes was measured after reaching secular equilibrium (at least 3.5 hours) by counting the γ-photon emissions of Bismuth-213 (²¹³Bi; the last radioactive daughter of ²²⁵Ac) using a γ-counter (Packard Cobra II Auto-Gamma, Model E5003).

To evaluate the extent of radioactivity retention vs. pH, liposomes encapsulating ²²⁵Ac-DOTA were incubated in cell-conditioned (following overnight incubation) and cell-containing media adjusted at different pH values. At different time points, liposome-containing aliquots were removed, passed through a Sephadex-G50 column eluted with PBS (1 mM EDTA, pH 7.4), and the radioactivity retained by liposomes was quantified as stated above.

### 2.3.3 Cell Lines

The MDA-MB-231 and MDA-MB-436 TNBC cell lines were purchased from ATCC and were cultured using Dulbecco's modified Eagle's media (DMEM) and Roswell Park Memorial Institute (RPMI), respectively, both supplemented with 10% FBS, 100 units/mL Penicillin and 100 μg/mL Streptomycin in an incubator at 37 C and 5% CO₂.

### 2.3.4 Colony Formation Assay

Cell monolayers were incubated for 6 hours at different pH values and varying radioactivity concentrations following overnight adhesion of 500,000 cells per well in 6-well plates. Upon completion of incubation, the wells were washed with PBS thrice, cells were carefully scraped and diluted in media (pH 7.4) at a concentration of 10,000 cells/mL, and were then plated in triplicate into tissue culture dishes at varying concentrations (3 cell densities for each radioactivity concentration). After about 10 doubling times (15 days and 17 days, for MDA-MB-231 and MDA-MB-436, respectively), the culture dishes were washed with water, colonies were fixed and stained using Crystal violet (0.05%) and 6% glutaraldehyde, respectively, and stained colonies were then counted. The survival fraction was determined by normalizing the counted number of treated colonies to the number of untreated colonies, accounting for the plating efficiency.

### 2.3.5 Spheroid Studies

Spheroids were formed by seeding 500 MDA-MB-231 cells (in Matrigel™ at 2.5% v/v) per well in 96-well round bottom plates coated with PolyHEMA. The plates were then centrifuged for 10 minutes at 1000 rcf and 4° C. Seven days after seeding, spheroids reached 400 μm-in-diameter at which point they were incubated with different treatments for 6 hours. Upon completion of incubation, treated spheroids were transferred into fresh media, and the spheroid volume (V=4*π*α*β²/3, where α and β are the major and minor diameters respectively) was monitored until the volume of the untreated spheroids reached an asymptote. At that point, the spheroids were then plated on adherent 96-well plates (one spheroid per plate) for evaluation of the extent of potential outgrowth. Once the untreated condition reached confluency, all spheroids were trypsinized and the numbers of cells were counted. The percent outgrowth was evaluated as the number of cells counted for each treatment normalized by the number of cells of the untreated condition.

### 2.3.6 Animal Studies

Mice were housed in filter top cages and provided with sterile food and water. Animal studies were performed per Institutional Animal Care and Use Committee protocol (IACUC). Four-to-six week old NSG (NOD scid gamma) female mice (JHU Breeding Facility), weighing around 20 g, were orthotopically inoculated with 500,000 MDA-MB-231 cells, suspended in 100 μL serum-free DMEM, into the second mammary fat pad by making a small incision on the right side of the mouse. Once the tumor volume reached approximately 25 mm³, 10-14 animals were randomly grouped into each treatment condition which was administered once, intravenously in 100 μL volume. Tumors volume was calculated using the ellipsoid formula (V=4*π*α*β²/3, where α and β were the major and minor diameters of the tumor, respectively) which were measured by a caliper. Animal weight was monitored daily and the animals were sacrificed when the conditions for euthanasia were met (ulceration or greater than 10% weight loss). At the endpoint of the study (14 days after treatment or earlier if conditions of euthanasia were met), the animals were scanned by MRI to detect axillary lymph node metastases. Upon completion of animal studies, tumors and critical organs were harvested and fixed in 10% neutral buffered formalin (NBF) for 24 hours. The blocks of tumors and organs were then grossed, processed, embedded, and sections were H&E stained for histological evaluation.

To determine the maximum tolerated dose (MTD, defined as the dose that gives <10% weight loss and no treatment-related deaths), tumor-free mice were treated at three different doses (3.7 kBq, 5.55 kBq and 7.4 kBq per animal).

### 2.3.7 Statistical Analysis

Results are reported as the arithmetic mean of n independent measurements±the standard deviation. ANOVA and Student's unpaired t-test were used to calculate significant differences in killing efficacy between the various constructs. p-values less than 0.05 were considered to be significant.

### 2.4 Results

### 2.4.1 Liposome Characterization

Table 2-2 shows that all liposomes, independent of the combinations of the release (R) and the adhesion (A) properties, had comparable sizes. When the titratable ‘adhesion lipid’ (DSPE-PEG2000-DAP) was included (compositions indicated by A+), the apparent zeta potential values were shifted towards more positive (R−A+) or less negative (R+A+) values with lowering pH due to protonation of the DAP moiety on the outer PEG-corona of the nanoparticles, as was demonstrated before. All liposome compositions exhibited comparable specific radioactivities (1.9-3.0 μCi/μmol of lipid). In the absence of the release property (R−), more than 80% of the loaded radioactivity (²²⁵Ac-DOTA) was stably retained by the carriers. Liposomes with the release property exhibited up to 40% release of the encapsulated ²²⁵Ac-DOTA with lowering pH to 6.0 at rates which were pH-independent (Table 2-3).

TABLE 2-2
Liposomes with different property combinations: characterization of Size, Zeta
Potential, ²²⁵Ac-DOTA Loading Efficiency, and Specific radioactivity. Errors correspond
to standard deviations of n = 4 independent liposome preparations.

| Liposome | | Specific activity | Size, nm | | Zeta Potential | Zeta Potential | Zeta Potential |
|---|---|---|---|---|---|---|---|
| properties | % Loading | (μCi/μmol lipid) | (PDI) | | pH 7.4 (mV) | pH 6.5 (mV) | 6.0 (mV) |
| R−A− | 75 ± 7 | 3.0 ± 0.3 | 114 ± 8 | (0.08 ± 0.01) | −3.7 ± 0.7 | −4.0 ± 0.9 | −3.5 ± 0.6 |
| R−A+ | 68 ± 6 | 2.7 ± 0.2 | 127 ± 12 | (0.10 ± 0.02) | 3.3 ± 1.0 | 4.1 ± 0.6 | 4.6 ± 0.6 |
| R+A− | 54 ± 8 | 2.2 ± 0.3 | 116 ± 14 | (0.10 ± 0.03) | −15.4 ± 2.8 | −15.5 ± 2.1 | −15.7 ± 1.0 |
| R+A+ | 47 ± 11 | 1.9 ± 0.4 | 116 ± 9 | (0.11 ± 0.03) | −5.0 ± 0.4 | −3.9 ± 0.4 | −3.3 ± 0.4 |

* indicates p-values < 0.05.

TABLE 2-3
Release kinetics of ²²⁵Ac-DOTA from liposomes of different property combinations.
Errors correspond to standard deviations of n = 3 independent liposome preparations.
y = y_{∞} + αe^{-bt}

| Liposome properties | pH | y_{∞} | a | b | t_{1/2} (h) |
|---|---|---|---|---|---|
| R−A− | 7.4 | 82 ± 4 | 17 ± 5 | 0.89 ± 0.45 | 0.94 ± 0.49 |
| | 7.0 | 82 ± 5 | 17 ± 5 | 2.49 ± 2.49 | 1.12 ± 1.54 |
| | 6.5 | 81 ± 6 | 17 ± 3 | 25.69 ± 41.45 | 1.37 ± 2.19 |
| | 6.0 | 82 ± 5 | 18 ± 5 | 3.78 ± 2.94 | 0.30 ± 0.26 |
| R−A+ | 7.4 | 85 ± 5 | 15 ± 6 | 116654.00 ± 202048.80 | 0.53 ± 0.54 |
| | 7.0 | 87 ± 4 | 13 ± 3 | 2.43 ± 2.61 | 0.62 ± 0.53 |
| | 6.5 | 81 ± 7 | 19 ± 7 | 2745680 ± 4755652 | 0.95 ± 1.53 |
| | 6.0 | 83 ± 3 | 16 ± 2 | 4.26 ± 4 42 | 0.35 ± 0.31 |
| R+A− | 7.4 | 82 ± 4 | 17 ± 2 | 1.65 ± 0.80 | 0.53 ± 0 34 |
| | 7.0 | 80 ± 2 | 19 ± 2 | 1.05 ± 0.28 | 0.69 ± 0.18 |
| | 6.5 | 67 ± 1 | 31 ± 2 | 1.10 ± 0.22 | 0.65 ± 0.12 |
| | 6.0 | 60 ± 4 | 38 ± 4 | 1.07 ± 0.24 | 0.79 ± 0.18 |
| R+A+ | 7.4 | 84 ± 3 | 16 ± 3 | 0.99 ± 0.14 | 0.71 ± 0.11 |
| | 7.0 | 84 ± 0 | 16 ± 1 | 1.01 ± 0.13 | 0.69 ± 0.08 |
| | 6.5 | 68 ± 4 | 31 ± 4 | 0.88 ± 0.05 | 0.79 ± 0.04 |
| | 6.0 | 63 ± 5 | 35 ± 5 | 0.80 ± 0.26 | 0.93 ± 0.29 |

### 2.4.2 Cell-Monolayer and Multicellular-Spheroid Studies

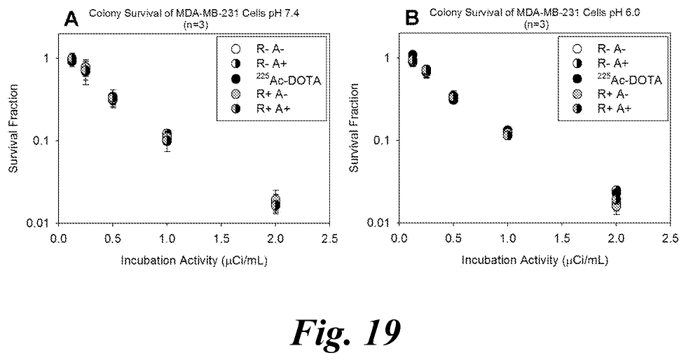

In the absence of transport-barriers, monolayers of the TNBC cell line MDA-MB-231 exhibited identical sensitivity to α-particle therapy (evaluated by colony formation, FIG. 19A-FIG. 19B) to all forms of liposomes encapsulating ²²⁵Ac-DOTA and to free ²²⁵Ac-DOTA. This response was expected since none of the liposomal forms exhibited any particular tendency to associate with cells. Stras et al., 2019. Also, not unexpectedly, sensitivity to α-particle therapy was independent of the extracellular pH (FIG. 19B) representing here the acidity in the tumor interstitium that triggers the release and adhesion properties on liposomes.

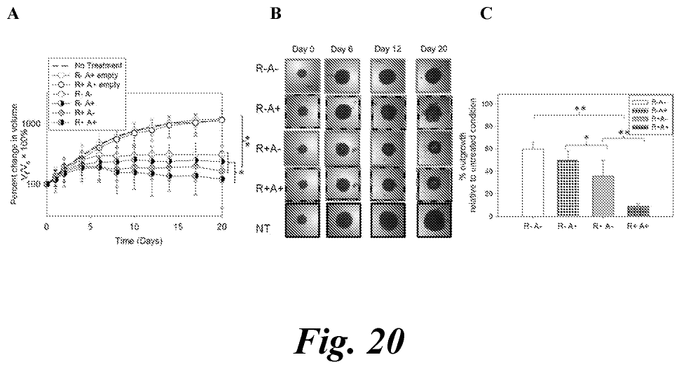

In spheroids, which were previously shown to develop interstitial pH gradients ranging from 6.3 to 7.4, Stras et al., 2019, it has previously been demonstrated the significance of diffusion-driven transport where the interstitial release played the dominant role in increasing the uniformity and delivered amounts of the time-integrated spatial profiles of drug surrogates. Zhu et al., 2017; Stras et al., 2019. Liposomes with both the release and adhesion properties (R+A+) best controlled the volume of MDA-MB-231 multicellular spheroids (FIG. 20A and FIG. 20B) and best inhibited the spheroid outgrowth (FIG. 20C, p-value<0.05)—used as surrogate of recurrence—followed by liposomes with the release property only (R+A−), and by the liposomes with the adhesion property only (R−A+). The non-radiolabeled liposomes did not have a measurable effect on the spheroid volume growth and/or outgrowth which was identical to the no-treatment condition.

### 2.4.3 In Vivo Studies

The I.V. injected liposomal radioactivity of 150 nCi per 20 gr mouse was found to be the MTD in tumor-free animals which are alive after 7.5 months. The H&E stained sections of the organs of tumor-free mice injected with liposomal radioactivity at 7.4 kBq (200 nCi), per 20 gr animal, that were euthanized on day 21 after administration of radioactivity included renal inflammation and splenic toxicity, but no noteworthy hepatic toxicity. Presence of neutrophils and macrophages in the kidneys could point to renal inflammation that may have been associated with mineral deposition. Spleen showed high hemosiderin deposition, depleted WBC count and no extramedullary hematopoiesis.

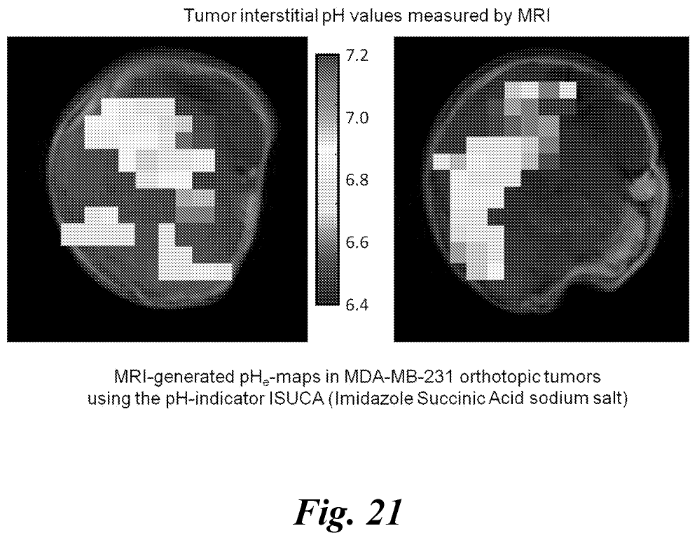

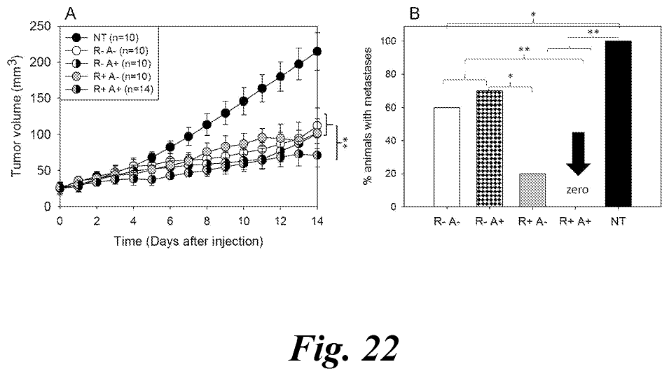

The orthotopic MDA-MB-231 orthotopic xenografts on the same mouse strain were confirmed by the MRI-generated pHₑ-maps in FIG. 21 to develop tumor interstitial acidity that ranged mostly between 6.6-6.8. Importantly, none of the animals which were administered ²²⁵Ac-DOTA in the form of R+A+ liposomes did develop spontaneous axillary lymph node metastases (FIG. 22B). Liposomes with the release property (independent of the ECM-adhesion property, R+A+ and R+A−) demonstrated the lowest (or no) occurrence of ALN metastases which were observed on all non-treated animals (p-value<0.05) and most of treated animals with the other two forms of liposomes without the release property.

### 2.5 Discussion

In this study, the delivery of highly-diffusive forms of ²²⁵Ac by carriers combining the properties of tumor interstitial release and adhesion on the tumors' ECM was demonstrated, so as to delay the carriers' clearance and to increase the time integrated tumor delivered dose. This characteristic not only inhibits progression of solid tumors but also delays the onset of spontaneous metastases. It seems more possible that the mechanism of delayed metastatic spreading is linked with the delivery action on the primary tumor sites and at lesser extent with action directly on the small (and probably still avascular) metastases, supporting a potential role of such therapeutics to be administered prophylactically.

Potential limitations of the proposed approach are: (a) the vascular permeability and (b) tumor acidity determining the tumor delivered dose and ultimately the pattern of tumor irradiation, respectively. While not all patient metastases exhibit vascular permeability to nanometer sized carriers, Prabhakar et al., 2013, when metastases do exhibit measurable liposome uptake, the extent of uptake is strongly and favorably correlated with tumor response of patients to liposome therapies. Lee et al., 2017. Lack of vascular permeability also may exclude antibody-based therapies due to size limitations. As mentioned in the introduction section, acidification of TNBC tumors is common and correlated to aggressive disease.

Kidney toxicity originating from escaped Bismuth-213, the last radioactive daughter of ²²⁵Ac, during blood circulation of liposomes was not observed in these experiments and was consistent with previous reports on different animal models utilizing liposomal delivery. Zhu et al., 2017. Splenic toxicity and secondary renal effects might be of concern for this approach, and are expected to define the MTD.

In summary, these observations demonstrate the potential of this ‘transport-oriented’ approach to lead to a new class of α-particle nanoradiotherapy as a platform technology to control tumor growth and/or spreading for difficult-to-treat solid tumors.

### Example 3

### Growth of Metastatic Triple Negative Breast Cancer is Inhibited by Deep Tumor-Penetrating and Slow Tumor-Clearing Chemotherapy: The Case of Tumor Adhering Liposomes with Interstitial Drug Release

### 3.1 Overview

The poor prognosis of Triple Negative Breast Cancer (TNBC) is attributed largely to the lack of tumor selective therapeutic modalities that effectively deliver lethal doses at the sites of metastatic disease. Tumor-selective drug delivery strategies that aim to improve uniformity in intratumoral drug microdistributions and to prolong exposure of these cancer cells to delivered therapeutics may improve therapeutic efficacy against established TNBC metastases.

The presently disclosed subject matter, in part, provides lipid carriers for selective (due to their nanometer size) tumor delivery which are loaded with cisplatin and are designed to exhibit the following properties when in the tumor interstitium: (1) interstitial drug release (for deeper tumor penetration of cisplatin); and/or (2) intratumoral/interstitial adhesion of the carriers to tumors' extracellular matrix (ECM)—not accompanied by cell internalization—for delayed tumor clearance of carriers prolonging cancer cell exposure to the cisplatin being released.

The presently disclosed subject matter demonstrates that on large multicellular spheroids, used as surrogates of avascular solid tumor regions, greater growth inhibition was strongly correlated with spatially more uniform drug concentrations (due to interstitial drug release) and with longer exposure to released drug (i.e. higher time-integrated drug concentrations enabled by slow clearing of adhesive nanoparticles). Lipid nanoparticles with both the release and adhesion properties were the most effective, followed by nanoparticles with only the releasing property, and then by nanoparticles with only the adhering property. In vivo, cisplatin-loaded nanoparticles with the releasing and/or the adhering properties significantly inhibited the growth of spontaneous TNBC metastases compared to conventional liposomal cisplatin, and the efficacy of different properties' combinations followed the same trends as in spheroids.

This Example demonstrates the therapeutic potential of a general strategy to bypass treatment limitations of established TNBC metastases due to lack of cell-targeting markers: aiming to optimize the temporal intratumoral drug microdistributions for more uniform and prolonged drug exposure.

### 3.2 Background

Triple Negative Breast Cancer (TNBC) accounts for 10-20% of breast carcinomas with the lowest 5-year survival rates among all breast cancer patients, Ovcaricek et al., 2011, due to high proliferation and reoccurrence outside the breast. Dawood, 2010; Dent et al, 2007. Metastatic TNBC is currently incurable. The poor prognosis, Dawood, 2010; Dent et al, 2007, in metastatic TNBC is attributed largely to the lack of tumor selective therapeutic modalities that effectively deliver lethal doses at the sites of disease. Fantini et al., 2012. A therapeutic modality that improves efficacy at the sites of TNBC metastases by better inhibiting tumor growth, without increasing toxicities, could have a significant clinical impact.

TNBC tumors frequently show sensitivity, Dawood, 2010; Anders et al., 2013, to platinum-derived agents, Telli, 2014, which have received extensive clinical use because of their DNA damaging activity. In the clinic, combination of platinum agents with experimental receptor-mediated targeted therapies designed to affect or inhibit key signaling pathways, Crown et al., 2012; Oakman et al., 2010, did not demonstrate statistically significant improvement following single-agent targeting approaches. Gelmon et al., 2012. Of lower toxicities but not yet with a significant improvement in therapeutic effect were also the clinical results of liposomal cisplatin (CDDP). Liu et al., 2013; van Hennik et al., 1987.

A strategy to increase the efficacy of delivered doses to established TNBC metastases could aim at (1) improving uniformity in intratumoral drug distributions, Minchinton and Tannock, 2006, and (2) prolonging exposure of these cancer cells to delivered therapeutics. It has previously been demonstrated in 3D multicellular spheroids (used as surrogates of the tumor avascular regions) that improved intratumoral uniformity can be enabled by drug nanoparticles engineered to release their (rapidly diffusing, due to small size,) therapeutic contents in the tumor interstitium enabling deep tumor-penetration of therapeutics. Stras et al., 2016; Zhu et al., 2017.

A key to this approach was shown to be the choice of drug nanoparticles that do not become internalized by cells—so as to maximize the fraction of released drug that may penetrate deeper in the tumor—and, to choose therapeutic agents (for example, cisplatin) which are efficiently transported across the cell membranes independent of the acidity of the local extracellular milieu. Stras et al., 2016. To effectively translate this strategy in vivo, however, the intratumoral residence times of such drug-loaded nanoparticles need to be prolonged to maximize the time-integrated dose delivered at the tumor. In this Example, to increase the nanoparticles' residence times in the tumors, an ‘adhesion switch’ is introduced on the nanoparticles' surface with the aim to slow down their tumor clearing kinetics. The switch is designed to promote nanoparticle adhesion on the extracellular matrix (ECM) while keeping their internalization by cells at a minimum.

Accordingly, the presently disclosed subject matter provides lipid-based nanoparticles (liposomes) loaded with cisplatin and exhibiting interstitial drug release and intratumoral adhesion. Both mechanisms affecting these properties were designed to be activated in the slightly acidic pH of the tumor interstitium (pH approximately 6.7-6.0). Helmlinger et al., 1997; Vaupel et al., 1989. In particular, for the tumor interstitial release, lipid nanoparticles were designed to contain pH-responsive lipid membranes forming reversible phase-separated lipid domains (resembling lipid patches) with lowering pH, as were reported previously. Bandekar et al., 2012; Karve et al., 2009; Karve et al., 2008; Karve et al., 2010. During circulation in the blood, these lipid nanoparticles comprise well-mixed, uniform membranes and stably retain their encapsulated contents. In the acidic tumor interstitium, occurrence of lipid-phase separation results in formation of lipid patches that span both lipid leaflets (via cross-bilayer registration). Bandekar and Sofou, 2012. This lipid rearrangement in the bilayer membrane can be utilized to create pronounced grain boundaries around the lipid domains enabling release of the encapsulated therapeutic agents which then—in a drug delivery setting—may diffuse deeper into solid tumors. Stras et al., 2016; Zhu et al., 2017. At the molecular level, lipid phase separation is enabled by balancing the permanent hydrogen-bonding attraction with the pH-tunable electrostatic repulsion between the lipids that form the domains (lipids with phosphatidyl serine headgroups, in this Example). The extent of membrane permeability on phase-separated bilayers was previously shown to be affected by the order of transient defects in the packing of lipid acyl-tails along the domain boundaries. Packing discontinuities along these boundaries may be enhanced by incorporation of saturated, gel-phase lipids with acyl-tails of different lengths. Karve et al., 2008.

The adhesion property—which is based on an electrostatic ‘switch’—is designed to attribute positive charge on the liposomes' outermost, undulating PEG-chain corona when in the tumor interstitum. This location of the switch, when in cationic form, still allows—as we demonstrate—for measurable liposome adhesion to the negatively charged ECM, Lieleg et al., 2009; Stylianopoulos et al., 2010, while it significantly suppresses the binding and internalization of liposomes by cells. This electrostatic switch is introduced on the lipid nanoparticles by the titratable chemical moiety dimethyl ammonium propane (DAP; with pKa between 6.58 and 6.81), Bailey and Cullis, 1994, which is zwitterionic during blood circulation of the nanoparticles and becomes cationic in the slightly acidic pH of the tumor interstitium. Helmlinger et al., 1997; Vaupel et al., 1989. As demonstrated herein, liposomes designed to contain the DAP-moiety directly (without a tether) on the headgroups of lipids, comprising the liposome membrane, exhibit strong cell binding and internalization when DAP is cationic. Contrary to this presentation of the cationic charge placed directly on the lipid nanoparticle's surface, in our design the titratable charge was placed on the free end of the undulating PEG-chains forming the liposome corona (FIG. 1). This surface architecture was hypothesized (and is demonstrated herein) to adequately conserve the electrostatic adhesion of nanoparticles on the tumors' ECM, therefore, significantly delaying the nanoparticle clearance from tumors while, importantly, keeping the nanoparticle association to and internalization by cells at a minimum. Tumor adhesion of nanoparticles without cell internalization is critical in our strategy which aims to delay the nanoparticle clearance from the tumor so as to increase the fraction of released drug that diffuses deeper in the interstitium of avascular tumor regions.

This Example characterizes on lipid nanoparticles the extent of the pH-dependent (interstitial) drug release property and the property of pH-dependent adhesion to the tumors' ECM. The role of these properties on affecting the transport of nanoparticles and their contents in 3D multicellular spheroids is quantified. The effect of the adhesion property on the biodistributions of lipid nanoparticles in tumor-bearing mice is evaluated and, finally, the significance of each property and their combination on controlling the growth of spheroids in vitro and of spontaneous TNBC metastases in vivo is evaluated.

### 3.3 Experimental Section

### 3.3.1 Materials

All lipid products were obtained from Avanti Polar lipids (Alabaster, USA) including 1,2-distearoyl-sn-glycero-3-phosphocoline (DSPC), 1,2-diarachidoyl-sn-glycero-3-phosphocholine (20PC), 1,2-dioctadecanoyl-sn-glycero-3-phospho-L-serine (sodium salt) (DSPS), 1,2-distearoyl-sn-glycero-3-phosphoethanolamine-N-[amino(polyethylene glycol)-2000](ammonium salt) (DSPE-PEG(2000)), and 1,2-dipalmitoyl-sn-glycero-3-phosphoethanolamine-N-(Lissamine Rhodamine B Sulfonyl) (Ammonium Salt) (DPPE-Rhodamine). The functionalized lipid 1,2-distearoyl-sn-glycero-3-phosphoethanolamine-N-PEG2000-dimethylammonium propanoyl (DSPE-PEG(2000)-DAP) was custom synthesized by Avanti Polar lipids. All materials are described in detail in the supplemental data.

### 3.3.2 Cell Lines

MDA-MB-231 and MDA-MB-436 TNBC cell lines were obtained from the American Type Culture Collection (ATCC, Rockville, MC, USA) and were cultured in DMEM Media and in RPMI 1640, respectively, both supplemented with 10% fetal bovine serum and 100 units/mL penicillin, and 100 mg/mL streptomycin at 37° C. with 5% CO₂. Mouse-derived MDA-MB-231 sublines were developed from primary and metastatic tumors formed in NOD scid gamma (NSG) female mice (vide infra) as described before. Iorns et al., 2012. Briefly, tumors were removed, ground, plated in petri dishes with DMEM media, and were grown for several weeks when only MDA-MB-231 subline cells survived which were then propagated and frozen. The following sublines (Table 1-1) were developed and studied: MDA-MB-231-PRI3 was derived from the (primary) orthotopic MDA-MB-231 xenograft tumor (PRI) of mouse numbered 3, the MDA-MB-231-ALN2 was derived from an axillary lymph node (ALN) metastasis from mouse numbered 2, and MDA-MB-231-LUNG1 was derived from lung (LUNG) surface metastases of mouse numbered 1. These metastatic sites were chosen due to their frequency in appearance: both ALN and lung metastases were detected in 45 out of 45 mice (100%).

### 3.3.3 Liposome Preparation and Loading of Cisplatin

The compositions of all lipid nanoparticles studied are listed on Table 1-2. All liposomes were PEGylated with 8 mole % DSPE-PEG(2000) lipid. The pH-triggered releasing property on lipid bilayers was introduced by combining a zwitterionic lipid (phosphatidyl choline) with a titratable anionic lipid (phosphatidyl serine) with four carbons difference in the lengths of their corresponding saturated acyl tails (20:0-PC and 16:0-PS, DPPS, respectively) (compositions numbered 3 and 5). The pH-triggered adhesion property on lipid nanoparticles was introduced by replacing the PEGylated lipid, DSPE-PEG(2000), with the ‘adhesion lipid’ DSPE-PEG(2000)-DAP (compositions numbered 2, 3 and 4). For the studies aiming to demonstrate the liposome properties bearing the ‘adhesion lipid’, DSPE-PEG(2000)-DAP, comparison was made to liposomes with ‘surface charge’, i.e. with the identical titratable (cationic) moiety DAP conjugated directly on the lipid headgroups, DSPE-DAP (composition numbered 1). All compositions were labeled with 0.125 mole % DPPE-Rhodamine lipid.

Lipid nanoparticles were prepared using the thin film hydration method. Briefly, the dry lipid film (10 to 80 μmoles total lipid) was hydrated with 1 mL of PBS (10 mM phosphate buffered saline with 1 mM EDTA) at pH 7.4, and this suspension was then annealed at 60° C. for 2 hours following extrusion for 21 times through two stacked 100 nm pore-sized polycarbonate membranes at 80° C. Cisplatin was then passively loaded into liposomes which were first passed through a Sepharose 4B column. Stras et al., 2016. After loading, unencapsulated cisplatin was removed from liposomes using a 10 cm Sephadex G50 column, eluted with PBS at pH 7.4. An aliquot of the collected liposome fraction was lysed with 0.5% Triton-X 100, and the platinum content was measured using a Graphite Furnace Atomic Absorption Spectrophotometer (GFAAS) (Buck Scientific, using a Hollow Cathode Pt 365.9 nm lamp) and quantified by comparison to a calibration curve as reported before. Stras et al., 2016. The lipid content was evaluated using the Stewart's Assay. Stewart, 1980. Before incubation with cells, all lipid nanoparticles were sterilized by filtration (200 μmeter filters, VWR, Radnor, Pa.).

### 3.3.4 Characterization of Lipid Nanoparticles

The size and zeta potential of lipid nanoparticles were determined using a Zetasizer Nano ZS 90 (Malvern, United Kingdom). Samples were diluted in PBS (10 mM phosphate buffer, 150 mM NaCl, 300 mOsm) for sizing or low salt PBS (10 mM phosphate buffer, 15 mM NaCl, 275 mM sucrose, 300 mOsm) for zeta potential measurement, respectively. Retention of cisplatin by nanoparticles was performed in 10% FBS-supplemented cell culture media in the presence of cells, for a 6-hour incubation period. At the end of incubation the liposome suspension (upon separation of cells) was run through a Sephadex G50 column to remove released cisplatin from liposomes, and the content of platinum in the collected fractions was quantified using the GFAAS.

### 3.3.5 Binding and Imaging Studies of Lipid Nanoparticles to Cells

For cell binding measurements, lipid vesicles labeled with 1.6 mole % DPPE-rhodamine lipid were incubated with cells in suspension at the ratio of 10⁶ liposomes per cell (0.2 mM total lipid and 0.8×10⁶ cells per mL). Aliquots were placed on ice (at 4° C.) or in a humidified incubator at 37° C. and 5% CO₂ for three hours to allow lipid vesicles to bind and/or become internalized by cells in suspension. Vesicles associated by cells were isolated by centrifugation, were resuspended in 750 μL of DI water, were sonicated for 10 minutes and finally were then mixed with acidified Isopropanol (10% HCl, 90% IPA) at 1:1: v/v ratio to ensure complete cell lysis before measurement of rhodamine's fluorescence intensity (ex/em: 550 nm/590 nm) using a spectrofluorometer (Fluorolog FL-1039/40, Horiba, Edison, N.J.).

For imaging, MDA-MB-231 cells were harvested with 0.05% Trypsin (w/w), and 100,000 cells were plated in 35 mm glass bottom dishes to adhere overnight. Cell monolayers were then incubated with 1.6 mole % DPPE-Rhodamine-labeled lipid vesicles at a ratio of 10⁶ liposomes per cell. After completion of incubation for 3 hours in media (DMEM) adjusted to pH 7.4 and 6.5, the cells were washed twice with PBS prior to Hoechst 33342 staining, and again washed twice with PBS. Z-stack images (step size=1 μm) of the cell monolayers were imaged using a Nikon A1 confocal laser scanning microscope (RFP for Rhodamine: excitation/emission wavelengths 561/595 nm; DAPI for Hoescht: excitation/emission wavelengths 405/450 nm) under an oil immersion 60× objective. The pH of media was adjusted by HCl, and the media were incubated in 37° C. with 5% CO₂ overnight for the pH to equilibrate before proceeding with any measurements.

### 3.3.6 Decellularization of Tumors

Tumors were harvested from mice bearing orthotopic MDA-MB-231 xenografts and were placed into tubes with 10 mL of 1% sodium dodecyl sulfate (SDS) dissolved in deionized water and supplemented with 1% Pen-Strep. This treatment has been shown to remove all cells, while leaving extracellular matrix (ECM) proteins fully intact, creating a decellularized ECM scaffold. Ott et al., 2008. The tubes were rotated until complete decellularization was achieved, with the SDS solution replaced every 24 hours. After approximately 3 to 4 days, depending on tumor size, complete decellularization was reached, marked by tumors turning completely white and translucent. The tumor ECM scaffolds were then washed several times with cold PBS to ensure the removal of SDS. Complete tumor decellularization was verified by imaging Hoechst stained tumor slices (30 μm thickness) before/after treatment to confirm no cells remained associated with the tumor ECM. Lü et al., 2014. Finally, the decellularized tumors were sliced into small pieces (˜10 mm³) and skewered on to stainless steel pins for imaging.

3.3.6 Clearance Profiles of Lipid Nanoparticles from Decellularized Tumor ECM

Tumor ECM scaffolds on-a-pin (as described above) were each placed into a 150 mm×25 mm petri dish with 200 mL of fresh PBS at pH 6.5. Once the tumor/pin was placed into the dish, the dish was not moved and fluorescent images were obtained every 10 minutes for 16 hours using an Olympus IX81 inverted fluorescence microscope with an 10× objective. The same section of the same tumor piece was measured for lipid vesicles with and without the titratable moiety DAP to account for any heterogeneities in the tumor sections. To analyze, the average intensity of the same region of a tumor piece was measured at each time point in ImageJ. The intensities were then normalized by the initial average intensity, and an exponential decay curve was fit to the data. Using this fitting, the areas under the curve and the half-lifes were calculated for the different liposome compositions on the same tumor piece and compared.

### 3.3.7 Evaluation of IC₅₀ Values on Cell Monolayers

To determine the IC₅₀ of liposomal cisplatin and of free cisplatin, 20,000 cells per well were plated in 96-well plates. A range of concentrations of sterile liposomes (containing or not cisplatin) or of free cisplatin was added to the wells mixed in media (with 10% FBS) at pH 7.4 and pH 6.5. Upon completion of a 6 hour incubation, cells were washed twice with PBS, and 10% FBS-supplemented fresh media was then added to wells. After two doubling times an MTT assay (Promega, Madison, Wis.) was used (following the vendor's instructions) to evaluate percent cell viability. Absorbance was read at 570 nm.

### 3.3.8 Multicellular Spheroid Formation and Characterization

Formation of MDA-MB-231 spheroids was described previously. Stras et al., 2016. To form spheroids using MDA-MB-231 mouse-derived sublines or the MDA-MB-436 cell line, cells were trypsinized and diluted in DMEM or RPMI 1640, respectively, with 2.5% (v/v) Matrigel™. Cells were plated at a density of 150-175 cells per well in polyHEMA coated U-shaped 96-well plates. Media, plates, and materials were kept at 4° C. to prevent gelation of Matrigel™. Plates were then centrifuged at 1000×g for 3 minutes to pellet cells, and after 10-11 days spheroids reached the desired size of 400 μm in diameter.

To determine the interstitial pH gradients, spheroids were incubated with SNARF-4F, a membrane impermeant pH indicator (ex: 488 nm, em: 580 nm and 640 nm) as described before, Stras et al., and also described in detail in the supplemental data.

3.3.9 Spatiotemporal Profiles of Lipid Nanoparticles and their Contents in Spheroids

To determine the uptake and clearance of lipid nanoparticles (liposomes) and their contents in spheroids, liposomes were prepared with 1 mole % DPPE-Rhodamine lipid and were loaded with CFDA-SE (ex/em: 497 nm/517 nm); final CFDA-SE concentration in liposome suspension was 800 nM. Averaged CFDA:Lipid mole ratios for all constructs were similar; 4.26×10⁻⁵±0.623×10⁻⁵: 1 (n=4).

Spheroids were incubated for 6 hours with liposomes labeled with DPPE-rhodamine and encapsulating CFDA-SE (ex/em: 497 nm/517 nm; used as a fluorescent surrogate for cisplatin) at 1 mM total lipid and 40 nM CFDA-SE, and upon completion of incubation spheroids were transferred to fresh media. At different time points (3 and 6 hours during incubation with liposomes; and 0.5, 1, 2, 4, and 24 hours following completion of incubation) several spheroids were sampled in a volume of 1 μL and frozen in Cryochrome gel at −80° C. Spheroids without any treatment were frozen to be used as background. The samples were then sliced on a cryotome at 20 μm thickness and the equatorial slices were imaged on an Olympus IX80 fluorescence microscope. To evaluate the radial fluorescence intensities in spheroid slices, images were analyzed using an in-house developed eroding code in Matlab averaging the intensity of each 5 μm-wide concentric ring of the spheroid as described before. Zhu et al., 2017. Calibration curves were evaluated using the same microscope on known concentrations of rhodamine-labeled liposomes and of CFDA-SE imaged in a quartz cuvette of optical pathlength identical to the thickness of the spheroid slices (20 μm). The spatial distributions were integrated over time (using the trapezoid rule) to express the time-integrated lipid concentrations or CFDA radial concentrations for each construct within spheroids.

### 3.3.10 Spheroid Treatment

Spheroids were incubated with different forms of cisplatin (liposomal or free) for 6 hours, washed once, and then moved to wells of fresh media. The % change in volume of spheroids over time (Vₜ/Vₒ×100) was monitored till the non-treated spheroids reached a plateau in growth. At that point the spheroids were plated on adherent cell culture 96-well plates (one spheroid per well) and were allowed to grow. When the control (non-treated) spheroids reached confluency, cells were trypsizined and counted using a Z1 Coulter Counter (Indianapolis, Ind.). The number of live cells was reported as % outgrowth relative to the counted numbers of non-treated cells.

### 3.3.11 Animal Studies

NSG female Mice (4-5 weeks old) were purchased from Johns Hopkins University Breeding Facility, and studies were performed per Institutional Animal Care and Use Committee protocol (IACUC). For orthotopic tumor inoculation, a small incision was made on each mouse, allowing injection of 0.5 million MDA-MB-231 cells suspended in 100 μL of serum-free cell culture media into the second mammary fat pad on the right side of animals.

For liposome biodistributions, ¹¹¹In-DTPA loaded liposomes were prepared as described before, Karve et al., 2009, and were injected intravenously in animals at doses of 7-12 μCi per animal. The exact injected activity into individual animals was determined by measuring each of the filled syringes in a dose calibrator and subtracting the residual activity post injection. At different time points, animals were sacrificed, blood was collected through a ventricle heart puncture, and tumors/organs of concern were harvested, weighted and their associated radioactivity was measured in a gamma counter (Packard Cobra II Auto-Gamma, Model E5003).

The metastatic animal model studied herein, Iorns et al., 2012, was found to be especially aggressive resulting in fast growth of tumor burden—mainly due to growth of the orthotopic tumor—demanding animal sacrifice only a few days after detection of metastatic sites by MRI imaging (usually 48±5 days after tumor inoculation). In order to allow for more time to potentially study the effect on the growth control of metastases of the different therapeutic modalities, the orthotopic xenografts were completely resected, and the growth of metastases was followed over time. This approach, of surgically removing the orthotopic tumor, prolonged the life expectancy of animals (up to 69±3 days after tumor inoculation, in the absence of treatment), and also better emulated the current clinical practice.

In particular, for efficacy studies on controlling the growth of mestastatic tumors, orthotopic tumors were removed surgically when they reached 160-200 mm³. When formation of metastatic tumors was confirmed by MRI (approximately 2.5 weeks after resection of the orthotopic xenograft), mice were treated with different types of liposomal cisplatin and with free cisplatin at the same dose of 7.5 mg/kg of cisplatin injected intravenously. Treatment groups consisted of 5-7 animals, and injections were performed three times in five-day intervals. The diameters of metastatic tumors at the start of therapy ranged from 0.5 mm to 2.0 mm. Metastatic tumor growth was monitored by MRI once a week over the course of the experiment, and on the day animals were euthanized. To determine change in volume of metastases, MRI images were analyzed using Vivoquant software (Invicro LLC, Boston, Mass.). Per IACUC protocol, animals were euthanized if they met conditions for euthanasia.

### 3.3.12 Statistical Analysis

Results are reported as the arithmetic mean of n independent measurements±the standard deviation. Student's unpaired t-test was used to calculate significant differences in killing efficacy between the various constructs. p-values less than 0.05 were considered to be significant.

For the in vivo studies on the control of metastatic tumor growth, groups were compared using the F statistic and the significance level was obtained using the exact null permutation distribution. Rosenbaum, 1984. This distribution was obtained by (i) repeatedly permuting the treatment labels to express the null hypothesis; and (ii) recalculating the F statistic for each such permutation. Then, the proportion of permutations with F≥the observed value was a correct p-value even if the variances varied across groups and the distribution was not normal.

### 3.4 Results

### 3.4.1 Characterization of DSPE-PEG(2000)-DAP Lipid (the ‘Adhesion’ Lipid)

The purity and molecular weight of the functionalized lipid DSPE-PEG(2000)-DAP were >99% and 2889.62 g/mol, respectively, as reported by Avanti Polar Lipids (details in FIG. 8A-FIG. 8C).

The rows numbered 1 and 2 of Table 1-3 show that on liposomes composed only of zwitterionic lipid headgroups (i.e., of phosphatidyl choline) addition of the titratable group DAP as DSPE-DAP (surface charge) and DSPE-PEG(2000)-DAP (charge on the free ends of PEG-chains, the ‘adhesion lipid’), respectively, resulted in more positive zeta potential values with lowering pH. The increase in zeta potential's value was attributed to the protonation of the DAP moiety (the pKa of DAP is reported to be between 6.58 and 6.81). Bailey and Cullis, 1994. Notably, liposome compositions numbered 3 to 6 (Table 1-2) exhibited negative values of the zeta potential due to the presence of the negatively charged lipid phosphatidyl serine (PS). On compositions 3 and 4, the protonation of DSPE-PEG(2000)-DAP lipid (attributing the adhesion property) resulted in significant changes in zeta potential to less negative values with lowering pH. The measured zeta potential values in these compositions were interpreted to indicate two protonation processes: first, the protonation of the anionic phosphatidyl serine (with apparent pKa of ˜6.5), Bajagur Kempegowda et al., 2009, resulting in zwitterionic moieties on the lipid headgroups, and the protonation of DSPE-PEG(2000)-DAP resulting in cationic charges—at a different plane far from the lipid headgroups—on the free ends of PEG-chains.

### 3.4.2 Lipid Nanoparticle Characterization

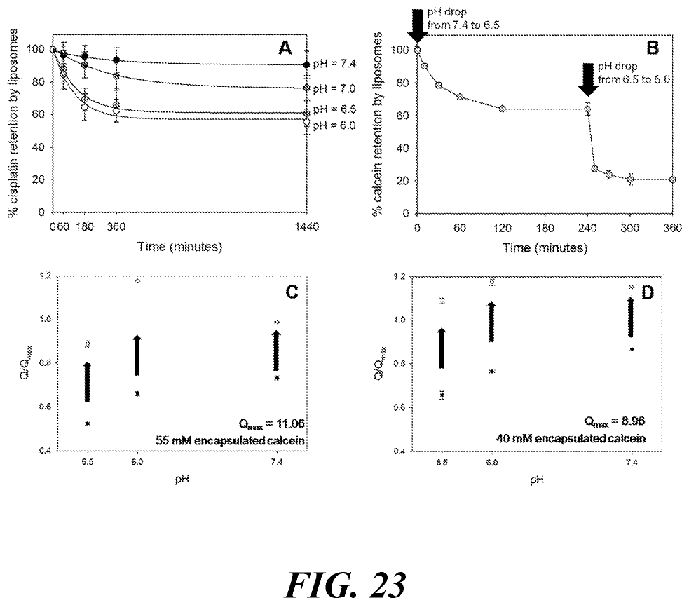

Liposomes, regardless of composition, had similar sizes (ranging from 109 to 121 nm), drug loading efficiencies and Drug-to-Lipid Ratios (Table 1-3). The pH-releasing liposomes (FIG. 23) exhibited significant release (approximately 15%) of encapsulated cisplatin at pH 6.5 (representing the average pH value of the acidic tumor interstitium) relative to pH 7.4 (representing the average pH during circulation in the blood) (p-values<0.01) independent of the presence or absence of DAP-functionalization. As expected by design, non-releasing liposomes stably retained the encapsulated cisplatin which was not affected by the pH acidification.

3.4.3 Lipid Nanoparticle Interactions with Cells and the Tumor ECM: Role of Location of the Titratable Cationic Moiety

The following studies demonstrated that moving the location of the titratable moiety DAP (becoming cationic in acidic pH) from the lipid membrane headgroups to the free ends of undulating PEG-chains (far from the lipid membrane surface) significantly reduced the attractive liposome interactions with cells (minimizing their internalization) but retained some extent of the adhesion of liposomes to the tumors' ECM so that a delayed liposome clearance from tumors was still preserved (vide infra).

In particular, liposomes functionalized with the DAP moiety directly attached on lipid headgroups using DSPE-DAP lipids (surface charge), indicated by composition 1 on Tables 3-1 and 3-2, exhibited—as expected—increased association with cells with protonation of the DAP moiety by lowering the extracellular pH from 7.4 to 6.5 as shown on FIG. 3A. Greater extents of liposome internalization also were observed when incubation with cells took place in acidic values of the extracellular pH. As hypothesized, moving the location of the titratable DAP group from the lipid headgroups (DSPE-DAP) to the free ends of PEG-chains, in the form of DSPE-PEG(2000)-DAP, resulted in a more than one order of magnitude decrease on the extent of liposomes which were adhered to and/or internalized by cells independent of the lipid membrane composition (FIG. 3B and FIG. 3C).

Confocal cell imaging confirmed that the location of cationic charge on liposomes can enhance (charge on lipid surface) or reduce (charge on end of PEG-chains) the extents of internalization by cells of DAP-containing liposomes, and additionally demonstrated pronounced differences of the type of interactions with cell membranes. Liposomes with protonated DAP directly conjugated on lipid headgroups (FIG. 3A, pH 6.5) exhibited localization at the cell plasma membranes which was not observed when liposomes were functionalized with DAP on the free ends of grafted PEG-chains.

Supporting of the goals of this study for intratumoral adhesion with low cell internalization, was the finding that localization of cationic DAP on the free ends of PEG-chains preserved to a significant extent the liposome attractive interactions with the tumors' extracellular matrix. In particular, upon incubation—in acidic conditions—with the decellularized extracellular matrix derived from MDA-MB-231 tumors from mice, liposomes functionalized with DSPE-PEG(2000)-DAP exhibited slower clearance kinetics compared to liposomes without a DAP-moiety. As expected, liposomes with the DAP moiety conjugated directly on their lipid-surface cleared with the slowest rate (FIG. 9).

Referring now to FIG. 9, the clearance profiles of liposomes without charge (containing only DSPE-PEG) and with the adhesion lipid (containing DSPE-PEG(2000)-DAP) were best fitted by a double exponential decay. The slow component of clearance was similar for both liposome types (half-lives of clearance were τ½=86±22 minutes and τ½=104±36 minutes, respectively). However, the fast component of clearance was significantly different (p-value=0.0004): only τ½=5±1 minutes for liposomes just functionalized with same fraction of PEG-chains and τ½=35±8 minutes for adherent liposomes. Additionally, the clearance profiles of liposomes with the same cationic charge directly on the lipid headgroups (containing DSPE-DAP) were best fitted by a single exponential decay with τ½=284±107 minutes.

The AUC values were 88±17, 129±37, and 337±80 for liposomes without charge (containing only DSPE-PEG; white symbols), for liposomes with the cationic charge located on the free ends of PEG-chains (containing DSPE-PEG(2000)-DAP, half-red/half-white symbols) and for liposomes with the cationic charge associated directly on the headgroup of lipids (containing DSPE-DAP; red symbols), respectively (p-values<0.05). Errors correspond to n=4 independent tumor samples and liposome preparations. The findings of the above characterization of liposome-cell and liposome-ECM interactions supported the term ‘adhesion lipid’ for DSPE-PEG(2000)-DAP.

### 3.4.4 Cell Monolayers: Effect of the Adhesion and Release Properties of Liposomal Cisplatin on IC₅₀ Values

Table 1-4 shows that pH-releasing liposomes loaded with cisplatin exhibited IC₅₀ values which were significantly lower at the acidic pH conditions relative to physiologic pH conditions. Non-releasing liposomes did not result in measurable IC₅₀ values at the conditions studied (see also FIG. 10 and FIG. 11). The killing efficacy of pH-releasing liposomes, as was shown before, is thought to be driven by the extracellularly released cisplatin (from liposomes) which then diffuses across the plasma membrane. Stras et al., 2016. The IC₅₀ values of free cisplatin were independent of the extracellular pH (Table 1-1).

Given the absence of significant cell internalization of liposomes functionalized with the adhesion lipid (DSPE-PEG(2000)-DAP) (FIG. 3), it was not unexpected that the IC₅₀ values of releasing liposomes did not depend on the presence or absence of PEG(2000)-DAP functionalization (Table 1-4). Liposomes not containing cisplatin did not result in significant cell kill (FIG. 10 and FIG. 11).

The TNBC cell line MDA-MB-436 was more sensitive to the platinum compound than the MDA-MB-231 line, in agreement with previous reports. Stefansson et al., 2012. The MDA-MB-436 is a BRCA-1 mutated TNBC cell line exhibiting aberrant DNA double-strand break repair mechanisms which to some extent have been the basis for increased clinical use of platinum-derived agents, Telli, 2014, against TNBC. Dawood, 2010; Anders et al., 2013. Interestingly, the MDA-MB-231 cell line and its animal derived sub-lines exhibited comparable drug sensitivities (not statistically different, Table 1-1) so the subsequent evaluation in spheroids of liposomal cisplatin forms was performed on the MDA-MB-231 (and on MDA-MB-436) as obtained from ATCC.

### 3.4.5 Multicellular Spheroids: Effect of the ECM-Adhesion and Drug-Release Properties of Nanoparticles on Drug Microdistributions and Killing Efficacy

The ECM-adhesion property (via inclusion of the DSPE-PEG(2000)-DAP in liposomes) increased significantly the time-integrated lipid concentrations (FIG. 4A) within spheroids partly due to delayed liposome clearance from spheroids (see FIG. 13). Comparison of the means of the AUCs (AUC within the spheroids) between compositions with the adhesion property (R+A+) and without the adhesion property (R+A−) indicated statistically significant difference (p-value<0.01). Importantly, the time-integrated concentrations (AUC within the spheroids) of the cisplatin surrogate (both encapsulated by liposomes and released, FIG. 4B) demonstrated higher values and more uniform microdistributions in the following order: AUC_{R+A+}>AUC_{R+A}−>AUC_{R-A}+>AUC_{R-A-} (p-value<0.01), where the property of adhesion to the ECM is indicated by “A⁺” or “A⁻” and the interstitial release property by “R⁺” or “R⁻”.

FIG. 5A-FIG. 5D show that the efficacy of liposomal cisplatin in controlling the growth and outgrowth (used as indirect surrogate of tumor recurrence) of TNBC spheroids was strongly correlated with the time-integrated microdistributions of cisplatin surrogates (AUC) shown in FIG. 4B. Accordingly, on spheroids formed by MDA-MB-231 and MDA-MB-436 cell lines (FIG. 5A-FIG. 5D), the inhibition of spheroid outgrowth by liposomal cisplatin followed the exact same order of the two properties' combinations shown above for the AUC of drug microdistributions from FIG. 4B: % Outgrowth Inhibition R+A+>% Outgrowth Inhibition R+A−>% Outgrowth Inhibition R−A+>% Outgrowth Inhibition R−A−. Liposomes not containing cisplatin did not affect spheroid growth and/or outgrowth (FIG. 14). The different sublines which were developed from MDA-MB-231 mouse-passaged tumors did not exhibit different IC₅₀ values to free cisplatin. In their spheroid form, some differences in sensitivity (not to the free cisplatin but) to the different liposomal forms could be attributed to the potential different cell packing densities within different cell-type spheroids which could, in principle, affect the efficacy of liposomes.

Both the adhesion and release properties were activated by the slightly acidic pH in the spheroid interstitium. The latter was confirmed in spheroids of all five cell lines with pH values decreasing from approximately 7.2-7.0 at the spheroid edge to average pH values of 6.2±0.1 towards the core (FIG. 12A-FIG. 12E).

### 3.4.6 Biodistributions of Lipid Nanoparticles: Effect of the Adhesion Property

FIG. 6A shows that the AUCₜᵤₘₒᵣ of I.V. administered liposomes with the adhesion property (containing DSPE-PEG(2000)-DAP; compositions designated “A⁺” in Tables 3-1, 3-2) was significantly greater than the AUCₜᵤₘₒᵣ of liposomes without the adhesion property (compositions designated “A⁻” in Tables 3-1, 3-2) (p-value<0.05). Importantly, the adhesion property did not significantly affect the blood clearance kinetics of liposomes (FIG. 6B), but delayed the uptake and clearance of liposomes from the liver and spleen, the two major off-target uptake sites (FIG. 6C and FIG. 6D), for reasons that are still not understood. The adhesion property did not affect the heart, lung and kidney uptake profiles (FIG. 15).

### 3.4.7 Growth Control of Spontaneous TNBC Metastases In Vivo

The volumes (vs. time) of the spontaneous, metastatic tumors are shown in FIG. 16 for each animal and are grouped per type of administered cisplatin-carrier. Because of the variable timing of metastatic onset, the variable metastatic tumor sizes, and the effectively short observation period due to the aggressiveness of the animal model, the single property that could collectively compare the effect of different therapies was identified to be the rate of tumor growth that is shown in FIG. 7.

The inhibition of the volume growth rates (AV/At, shown in FIG. 7) of the right axillary lymph node metastases (ALN), the dominant spontaneous metastases on this animal model, treated with liposomal cisplatin exhibiting different combinations of the release and adhesion properties, followed the same trend that was observed on the growth control studies of spheroids: ΔV/Δt_{R+A+}≤ΔV/Δt_{R+A-}<ΔV/Δt_{R-A+}<ΔV/Δt_{R-A-} In particular, the ALN tumor growth was significantly slower when animals were treated with liposomal cisplatin bearing at least the release property compared to liposomal cisplatin without any of the two properties (p-values<0.05 from t-test) and also compared to no treatment (supported also by the summary statistics, Table 3-1, and the ANOVA on all groups and on subgroups {NT or R−A−}vs. {any group with at least R+ or A+}, Table 3-2). Growth rates were calculated, to a first approximation without limiting accuracy, from the slopes of linear functions fitting the measured ALN volumes over time (FIG. 16). The change in tumor volume (slope) was calculated for individual mice, and was then averaged for each treatment group.

TABLE 3-1
Summary statistics

| Groups | n | Average | SD |
|---|---|---|---|
| NT | 7 | 17.1 | 4.1 |
| R+A+ | 5 | 11.2 | 3.3 |
| R−A+ | 7 | 15.4 | 3.9 |
| R+A− | 5 | 13.6 | 3.7 |
| R−A− | 6 | 22.3 | 4.7 |

TABLE 3-2
ANOVA for all groups and after subgrouping
{NT or R−A−} vs. {any R+ or A+}

| subgrouping: | F-statistic (d1, d2) | p-value |
|---|---|---|
| all groups possibly | 1.84 (4, 25) | 0.161 |
| different | | |
| {NT or R−N−} vs. | 4.85 (1, 28) | 0.038 |
| {any R+ or A+} | | |

Free cisplatin, although more effective in spheroids, resulted in the shortest animal survival, and 100% of animals were euthanized because of weight loss possibly attributed to acute toxicities (FIG. 17) for injected doses (7.5 mg/Kg) which were, however, equal to the reported MTD. Leite et al., 2012. The end-point justification for the majority of animals treated with liposomal cisplatin was due to ulceration and/or tumor burden.

### 3.5 Discussion

The therapeutic efficacy of delivered agents against established TNBC metastases could be improved by more uniform intratumoral drug distributions, Minchinton and Tannock, 2006; Stras et al., 2016; Zhu et al., 2017, combined, with prolonged exposure of cancer cells to these agents. Together, these factors may cooperatively improve the drug's tumor microdistributions increasing the population of tumor cells exposed to lethal doses for longer time, therefore, potentially delaying the growth of these tumors.

The intratumoral temporal microdistributions of nanoparticle-delivered therapeutics depend on the effective diffusion times of the carriers in the tumor interstitium and on the binding/internalization times of the carriers to cancer cells within the tumors. When the time scales of the above two processes are comparable, then the drug microdistributions within tumors are heterogeneous (for relatively limited blood circulation times of the nanoparticles) and become even more so by the increased tumor pressures, Heldin et al., 2004, which further obstruct interstitial diffusion.

To improve uniformity of drug distributions within established tumors, we previously reported, Stras et al., 2016; Zhu et al., 2017, an approach relying on the release of the free forms of therapeutics within the tumor interstitium from nanoparticles which were designed to not become internalized by cells. For therapeutic agents, such as cisplatin, which diffuse across the cell plasma membrane independent of the interstitial pH, keeping the extent of drug carrier internalization by cells to a minimum is critical in enabling intratumoral uniformity of the microdistributions of therapeutic agents in solid tumors. Otherwise, although counterintuitive, ‘consumption’ of drug carriers by cell endocytosis would decrease their population in the interstitial space and would limit the amount of free drug that would be available to penetrate into solid tumors. To effectively translate this strategy in vivo, however, the intratumoral residence times of such drug-loaded nanoparticles should be adequately long to enable prolonged exposure of cancer cells to the agents being released.

To delay the clearance of nanoparticles from tumors without being internalized by cancer cells, lipid nanoparticles (liposomes) were functionalized with an ‘adhesion switch’ on their surface. The choice of the particular titratable moiety (DAP) as the adhesion switch was based on its property to render cationic the liposomes' PEG-outermost-corona, as opposed to the liposomes' lipid headgroups, when liposomes experienced the slightly acidic pH of the tumor interstitium. Helmlinger et al., 1997; Vaupel et al., 1989. This behavior was supported by the in vivo findings showing that the nanoparticles with the adhesion property exhibited identical blood clearance kinetics but greater AUC in tumors compared to nanoparticles which were simply PEGylated not containing the DAP-moiety. Although we do not have direct evidence of the potential adhesion/adsorption of proteins onto the PEG-DAP corona of these nanoparticles when in vivo and how this process could have affected the electrostatic interactions with the biological milieu, the in vitro studies which were conducted in the presence of serum proteins, demonstrated an explicit effect of electrostatics on cell- and ECM-adhesion.” The location of the cationic charge was key on the design of this adhesion switch:

nanoparticles functionalized with DAP on the free ends of the PEG-chains (in the form of DSPE-PEG(2000)-DAP) exhibited minimal adhesion/adsorption to cancer cells compared to nanoparticles functionalized with DAP directly on their lipid headgroups. In the latter case, the attractive cationic charge is directly located on the liposome membrane surface, therefore, the electrostatic contact results in close apposition of the liposome membrane and the cell plasma membrane. In the former case, the attractive cationic charge is located at the free undulating end of the PEG chains. Therefore, when the electrostatic contact occurs it is between the ends of the PEG chains (on liposomes) and the cell plasma membrane. In this scenario, the PEG-chains may behave as a steric barrier opposing the close apposition of the liposomes' lipid headgroups and the cells' plasma membrane minimizing their attractive interactions. Furthermore, attempts to measure the desorption kinetics of the cell-adsorbed lipid nanoparticles (when the DSPE-PEG(2000)-DAP moiety was fully protonated) resulted in findings that suggested too fast desorption rates (with half-lives shorter than 30 minutes which is the resolution of the measurement method used). These relatively fast desorption kinetics could have also contributed to the limited internalization by cells for which the kinetics of (receptor-mediated) endocytosis of nanoparticles are of the order of 30 min (half-life of cell-bound nanoparticle internalization). Sempkowski et al., 2016.

The zeta potential alone is not an adequate property to characterize the charge location on a particle. However, the charge location plays a key role on the interactions of particles with the biological milieu (cells, the ECM). In particular, as shown on Table 1-3, although both compositions 1 and 2 had a similar zeta potential, for liposomes of composition 1 the cationic charge was directly located on the lipid headgroups and for liposomes of composition 2 the cationic charge was located on the free ends of PEG chains, resulting in different interactions of the two liposome compositions with cells (see FIG. 3A for composition 1, and FIG. 3B for composition 2, or FIG. 3D where liposomes of composition 1 exhibited the slowest clearance from the ECM followed by liposomes of composition 2). A potential explanation of the observed differences is given on the previous paragraph. Additionally, the zeta potential is a collective measurement which does not differentiate between distinguished planes of opposite charges on the same particle. For example, compositions 3 and 4, which also contained the DSPE-PEG-DAP lipid as did composition 2, exhibited a negative value for zeta potential (shown on Table 1-3) as opposed to the positive value of zeta potential measured for composition 2. This was because compositions 3 and 4 also contained phosphatidyl serine which has a negative charge and was located directly on their lipid headgroups (in other words, located at least 3.4 nm far from the PEG-chains on the end of which the cationic DAP was located). Bajagur Kempegowda et al., 2012; de Gennes, 1980. Both compositions 3 and 4 (FIG. 3C), and composition 2 (FIG. 3B), however, exhibited similar (low) uptake by the cancer cells due to, we postulate, the same amount of DSPE-PEG-DAP lipid that projected a similar density of cationic charge on the outermost PEG-corona of the liposomes in all cases.

The development of delivery carriers targeting the tumor extracellular matrix (ECM) using specific ligands has been recently discussed by Raavé et al, 2018. The difference of these designs to the current approach is that the latter exhibits increasing tendency to associate with the ECM as the tumor interstitial pH becomes more acidic. In principle, if the tumor acidity increases at depths farther from the neovasculature, Stras et al., 2016, the latter approach of gradually increasing adhesion with penetration depth may not further reduce the effective penetration depths of the carriers themselves which could be the case of functionalized nanoparticles with significant affinity. Lee et al., 2010. Since intratumoral pH gradients are far from ideal, however, in practice these differences may not be observed.

The adhesion property on the pH-releasing liposomes has the potential to increase the amount of the released therapeutic delivered within the avascular regions of tumors for the following reasons. First, the release process of contents from these liposomes is not instantaneous; the half-life of content release from these liposomes is of the order of 90 minutes for pH-values comparable to the pH of the tumor interstitium (FIG. 23A). Second, pH-releasing liposomes release their contents in an all-or-none mode (FIG. 23B). In other words, at a given acidic pH there is a specific subpopulation of liposomes that release all of their contents. Then, if the pH is further acidified on the same liposome suspension, yet another fraction of the liposome population will release all of their contents, and so on. The adhesion property on these liposomes, which was shown to increase their residence times in tumors, may potentially increase the probability that these liposomes would experience microenvironments with more acidic pH so as to collectively release even more of their therapeutic contents.

TABLE 3-3
Single Exponential Decay Fitting of cisplatin release
f(t) = yₒ + α*exp(−ln2*t/τ_{1/2})

| pH | yₒ | α | τ_{1/2} (mins) |
|---|---|---|---|
| 7.4 | 90.5 ± 4.3 | 8.7 ± 3.9 | 200.5 ± 3.4 |
| 7 | 76.0 ± 4.1 | 24 ± 3.8 | 210.3 ± 10.2 |
| 6.5 | 61.2 ± 3.1 | 39.4 ± 5.8 | 91.3 ± 8.2 |
| 6.0 | 57.3 ± 3.9 | 38.1 ± 2.6 | 84.6 ± 6.4 |

Liposomal encapsulation of cisplatin was previously shown to provide partial relief of the toxicities of the free agent. Leite et al., 2012; Sempkowski et al., 2014. In this study, we demonstrated that in addition to controlling toxicities (relative to free cisplatin), liposomal carriers with fast interstitial drug release and/or slow tumor clearance had the potential to significantly suppress the growth rates of spontaneous TNBC metastases in vivo relative to liposomes that did not exhibit any of these two properties. The animal model used in this study was found to be particularly aggressive (vide infra) to allow us to identify effects of the different property combinations on the duration of animal survival. Although the use of spheroids to capture the tumor interstitial pH gradients, transport limitations and ECM environment is by default limited, the trends in transport-response dependences that were observed in spheroids were also observed in the ability of nanoparticles with different property combinations to control the growth rate of metastases in vivo. In animals, unlike the well-controlled spheroid studies, liposomes with any of the release (R) or the adhesion (A) properties were better in controlling tumor growth than liposomes without any of the two properties and better than the no treatment, however, there was no statistical difference between liposomes with different combinations of R and A. This could be attributed to the potentially faster growth rates of the spontaneous metastatic tumor(s) in vivo relative to the killing kinetics of cisplatin, to the choice of the level of administered dose, and/or to the heterogeneities of the metastatic tumors themselves both in terms of sensitivity to the therapeutic and to the transport of liposomes. Lastly, but not least, in this study, it was not the primary, well-defined inoculated tumors that were monitored, but the spontaneous metastases which varied on their time of appearance and, therefore, their size at the time of therapy initiation. All of the above factors set the potential limitations of the applicability of the current approach.

In vivo, the property of interstitial release played a major role in controlling tumor growth whereas the adhesion of liposomes to the tumors' ECM exhibited a secondary role (although it clearly increased the tumor AUC (FIG. 6A)). It is possible, therefore, that for the aggressively growing metastatic tumors that were studied in the particular animal model, and for the particular therapeutic (cisplatin), the additional amount of cisplatin that was released in the tumor due to the longer residence time of adhering liposomes in the tumors may have not been high enough to result in further tumor growth inhibition. The aggressiveness of the particular animal model was also confirmed by histopathology.

To summarize, the main reason of death for all animals was the uncontrollable tumor burden with the exception of the acute deaths of animals which were treated with free cisplatin. The histopathology evaluation reported that mice carried significant tumor burden especially in the liver and lungs. Tumors (or tumor emboli) were associated with necrosis in several sites, and were mostly evident in the liver (FIG. 18).

Finally, the relevance of nanoparticle-based cancer therapies to human disease has been extensively discussed and debated. Prabhakar et al., 2012. The measurable uptake of lipid nanoparticles by tumors is described by the so called EPR effect. It is well understood that not all human established metastases exhibit such behavior, but when they do exhibit uptake of nanoparticles then this uptake is strongly and favorably correlated with tumor response to nanoparticle therapies, as is clinically proven. Lee et al., 2017. An additional point that is relevant to our approach involves the acidification of the intratumoral pH (as it relates to the two key properties of nanoparticles: release and adhesion) which is also common in human TNBC, Basu et al., 2008, and has been correlated with highly aggressive tumors. Vaupel et al., 1989; Estrella et al., 2013; Vaupel et al., 2004. Selection of patients may be conducted by personalizing nanomedicine, Wang, 2015; Shi et al., 2017, with biomarkers, in this particular case, the intratumoral microdistributions of probe-like carriers and of the tumor acidity.

In summary, without wishing to be bound to any one particular theory, it was thought that growth inhibition of established TNBC metastases could be improved by enhancing the uniformity of drug microdistributions within the tumors and by prolonging the exposure of cancer cells to the delivered therapeutics. Herein it was demonstrated that a way to achieve this is by I.V. administration of lipid nanoparticles loaded with cisplatin exhibiting cisplatin release in the tumor interstitium (for deeper tumor penetration) while nanoparticles are designed to not become internalized by cancer cells but to, instead, adhere to the tumor ECM for enabling longer residence times within the tumors for release of more drug.

### 3.6 Supporting Information

### 3.6.1 Materials and Methods

### 3.6.1.1 Materials

In addition to lipids described in the main text, cholesterol, cis-diamminedichloroplatinum (II) (CDDP), phosphate buffered saline (PBS) tablets, Sephodex G-50 resin, Sepharose 4B resin, and chloroform were purchased from Sigma-Aldrich Chemical (Atlanta, Ga.). Polycarbonate membranes (100 nm pore size) for extrusion, and extruder setups were purchased from Avestin (Ottawa, ON, Canada). EthylenediamineTetraacetic Acid, Disodium Salt Dihydrate (EDTA) and SNARF-4F were purchased from Thermo Fisher Scientific (Waltham, Mass.). Filters used for sterilization, with 200 micron pore diameters, were purchased from VWR (Radnor, Pa.).

Media was purchased from ATCC, fetal bovine serum was purchased from Omega Scientific (Tarzana, Calif.) and penicillin streptomycin was purchased from Fisher Scientific (Waltham, Mass.). Matrigel™ used in the formation of multicellular spheroids was also purchased from Fisher Scientific.

3.6.1.2 Measurement of Interstitial pH Gradients (pHₑ) in Spheroids

To determine the interstitial pH gradients, spheroids, at a size of 300±30 μm, were incubated for 12 hours with SNARF-4F, a membrane impermeant pH indicator (ex: 488 nm, em: 580 nm and 640 nm) whose ratio of intensities in the red and the green channels were shown to be pH dependent. Stras et al., 2016. Upon completion of incubation, spheroids were washed and placed in wells of fresh media for imaging using a Zeiss LSM510 Laser Scanning Confocal Microscope. Ten micrometer thick z-stacks were obtained through the entirety of the spheroid to allow identification of the equatorial optical slice on which an in-house erosion algorithm was used to calculate the average intensities in both the green and the red channel on 5 μm-wide concentric rings from the edge of the optical slice to the core. The fluorescent intensities of ring averaged intensities on equatorial slices of spheroids not incubated with SNARF-4F were subtracted from the above fluorescent images to correct for background intensities. A calibration curve of the ratios of the SNARF-4F intensities (acquired with the same microscope) in the red and green channels in media of known pH values was used to correlate the spheroid radial red/green average ratios to the spheroids' interstitial pH (pHₑ).

### REFERENCES

All publications, patent applications, patents, and other references mentioned in the specification are indicative of the level of those skilled in the art to which the presently disclosed subject matter pertains. All publications, patent applications, patents, and other references are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Anders, C. K.; Zagar, T. M.; Carey, L. A. The management of early-stage and metastatic triple-negative breast cancer: a review. Hematology/oncology clinics of North America 2013, 27, (4), 737-49, viii.
Auguste, D. T.; Armes, S. P.; Brzezinska, K. R.; Deming, T. J.; Kohn, J.; Prud'homme, R. K. pH triggered release of protective poly(ethylene glycol)-b-polycation copolymers from liposomes. Biomaterials 2006, 27, (12), 2599-2608.
Bailey, A. L.; Cullis, P. R. Membrane fusion with cationic liposomes: Effectes of target membrane lipid composition. Biochemistry 1997, 36, 1628-1634.
Bailey, A. L.; Cullis, P. R. Modulation of Membrane Fusion by Asymmetric Transbilayer Distributions of Amino Lipids. Biochemistry 1994, 33, (42), 12573-12580.
Bajagur Kempegowda, G.; Karve, S.; Bandekar, A.; Adhikari, A.; Khaimchayev, T.; Sofou, S. pH-dependent formation of lipid heterogeneities controls surface topography and binding reactivity in functionalized bilayers. Langmuir 2009, 25, 8144-8151.
Bandekar, A.; Karve, S.; M.-Y., C.; Mu, Q.; Rotolo, J.; Sofou, S. Antitumor efficacy following the intracellular and interstitial release of liposomal doxorubicin. Biomaterials 2012, 33, 4345-4352.
Bandekar, A.; Sofou, S. Floret-shaped solid domains on giant fluid lipid vesicles induced by pH. Langmuir 2012, 28, 4113-4122.
Basu, S.; Chen, W.; Tchou, J.; Mavi, A.; Cermik, T.; Czerniecki, B.; Schnall, M.; Alavi, A. Comparison of triple-negative and estrogen receptor-positive/progesterone receptor-positive/HER2-negative breast carcinoma using quantitative fluorine-18 fluorodeoxyglucose/positron emission tomography imaging parameters. Cancer 2008, 112, (5), 995-1000.
Bhagat, M.; Halligan, S.; Sofou, S. Nanoparticles as drug carriers to solid tumors: considerations on tumor penetration and exposure of tumor cells to therapeutic agents. Current Pharm Biotechnology 2012, 13, 1306-1316.
Crown, J.; O'Shaughnessy, J.; Gullo, G. Emerging targeted therapies in triple-negative breast cancer. Annals of Oncology 2012, 23, (suppl 6), vi56-vi65.
Dawood, S. Triple-negative breast cancer: epidemiology and management options. Drugs 2010, 70, (17), 2247-58.
de Gennes, P. G. Conformations of polymers attached to an interface. Macromolecules 1980, 13, 1069-1075.
Dent, R.; Trudeau, M.; Pritchard, K. I.; Hanna, W. M.; Kahn, H. K.; Sawka, C. A.; Lickley, L. A.; Rawlinson, E.; Sun, P.; Narod, S. A. Triple-negative breast cancer: clinical features and patterns of recurrence. Clin Cancer Res 2007, 13, (15 Pt 1), 4429-34.
Estrella, V.; Chen, T.; Lloyd, M.; Wojtkowiak, J.; Cornnell, H. H.; Ibrahim-Hashim, A.; Bailey, K.; Balagurunathan, Y.; Rothberg, J. M.; Sloane, B. F.; Johnson, J.; Gatenby, R. A.; Gillies, R. J. Acidity Generated by the Tumor Microenvironment Drives Local Invasion. Cancer Research 2013, 73, (5), 1524-1535.
Fantini, M.; Santelmo, C.; Drudi, F.; Ridolfi, C.; Barzotti, E.; Gianni, L.; Arcangeli, V.; Affatato, A.; Ravaioli, A. Triple Negative Breast Cancer Treatment: Use of Platinum and Platinum Analogs. Journal of Cancer Therapy 2012, 3, 777-781.
Fournier, C.; Zahnreich, S.; Kraft, D.; Friedrich, T.; Voss, K. O.; Durante, M.; Ritter, S. The Fate of a Normal Human Cell Traversed by a Single Charged Particle. Scientific Reports 2012, 2, 643.
Gelmon, K.; Dent, R.; Mackey, J. R.; Laing, K.; McLeod, D.; Verma, S. Targeting triple-negative breast cancer: optimising therapeutic outcomes. Annals of Oncology 2012, 23, 2223-34.
Heldin, C.-H.; Rubin, K.; Pietras, K.; Ostman, A. High interstitial fluid pressure an obstacle in cancer therapy. Nature Reviews Cancer 2004, 4, 806-813.
Helmlinger, G.; Yuan, F.; Dellian, M.; Jain, R. K. Interstitial pH and pO2 gradients in solid tumors in vivo: high-resolution measurements reveal a lack of correlation. Nature medicine 1997, 3, 177-182.
Humm, J. L. A microdosimetric model of astatine-211 labeled antibodies for radioimmunotherapy. Int J Radiat Oncol Biol Phys 1987, 13, 1767-1773.
Humm, J. L.; Chin, L. M. A model of cell inactivation by alpha-particle internal emitters. Radiat Res 1993, 134, 143-150.
Iorns, E.; Drews-Elger, K.; Ward, T. M.; Dean, S.; Clarke, J.; Berry, D.; Ashry, D. E.; Lippman, M. A New Mouse Model for the Study of Human Breast Cancer Metastasis. PLoS ONE 2012, 7, (10), e47995.
Karve, S.; Alaouie, A.; Zhou, Y.; Rotolo, J.; Sofou, S. The use of pH-triggered leaky heterogeneities on rigid lipid bilayers to improve intracellular trafficking and therapeutic potential of targeted liposomal immunochemotherapy. Biomaterials 2009, 30, 6055-6064.
Karve, S.; Bajagur Kempegowda, G.; Sofou, S. Heterogeneous domains and membrane permeability in phosphatidylcholine-phosphatidic acid rigid vesicles as a function of pH and lipid chain mismatch Langmuir 2008, 24, 5679-5688.
Karve, S.; Bandekar, A.; Ali, M. R.; Sofou, S. The pH-dependent association with cancer cells of tunable functionalized lipid vesicles with encapsulated doxorubicin for high cell-kill selectivity. Biomaterials 2010, 31, 4409-4416.
Kratochwil, C.; Bruchertseifer, F.; Giesel, F. L.; Weis, M.; Verburg, F. A.; Mottaghy, F.; Kopka, K.; Apostolidis, C.; Haberkorn, U.; Morgenstern, A. 225Ac-PSMA-617 for PSMA targeting alpha-radiation therapy of patients with metastatic castration-resistant prostate cancer. Journal of Nuclear Medicine 2016, 57, 1941-1944.
Kratochwil, C.; Bruchertseifer, F.; Rathke, H.; Bronzel, M.; Apostolidis, C.; Weichert, W.; Haberkorn, U.; Giesel, F. L.; Morgenstern, A. Targeted α-Therapy of Metastatic Castration-Resistant Prostate Cancer with 225Ac-PSMA-617: Dosimetry Estimate and Empiric Dose Finding. Journal of Nuclear Medicine 2017, 58, (10), 1624-1631.
Lee, H.; Fonge, H.; Hoang, B.; Reilly, R. M.; Allen, C. The Effects of Particle Size and Molecular Targeting on the Intratumoral and Subcellular Distribution of Polymeric Nanoparticles. Molecular Pharmaceutics 2010, 7, 1195-1208.
Lee, H.; Shields, A. F.; Siegel, B. A.; Miller, K. D.; Krop, I.; Ma, C. X.; LoRusso, P. M.; Munster, P. N.; Campbell, K.; Gaddy, D. F.; Leonard, S. C.; Geretti, E.; Blocker, S. J.; Kirpotin, D. B.; Moyo, V.; Wickham, T. J.; Hendriks, B. S. 64Cu-MM-302 Positron Emission Tomography Quantifies Variability of Enhanced Permeability and Retention of Nanoparticles in Relation to Treatment Response in Patients with Metastatic Breast Cancer. Clinical Cancer Research 2017.
Leite, E. A.; Lana, A. M.; Junior, A. D.; Coelho, L. G.; De Oliveira, M. C. Acute toxicity study of cisplatin loaded long-circulating and pH-sensitive liposomes administered in mice. J Biomed Nanotechnol 2012, 8, 229-139.
Lieleg, O.; Baumgartel, R. M.; Bausch, A. R. Selective Filtering of Particles by the Extracellular Matrix: An Electrostatic Bandpass. Biophysical Journal 2009, 97, (6), 1569-1577.
Lin, J.; Alexander-Katz, A. Cell Membranes Open “Doors” for Cationic Nanoparticles/Biomolecules: Insights into Uptake Kinetics. ACS Nano 2013, 7, (12), 10799-10808.
Liu, D.; He, C.; Wang, A. Z.; Lin, W. Application of liposomal technologies for delivery of platinum analogs in oncology. International journal of nanomedicine 2013, 8, 3309-19.
Lü W.-D.; Zhang, L.; Wu, C.-L.; Liu, Z.-G.; Lei, G.-Y.; Liu, J.; Gao, W.; Hu, Y.-R. Development of an Acellular Tumor Extracellular Matrix as a Three-Dimensional Scaffold for Tumor Engineering. PLOS ONE 2014, 9, (7), e103672.
Macklis, R. M.; Kinsey, B. M.; Kassis, A. I.; Ferrara, J. L.; Archer, R. W.; Hines, J. J.; Coleman, C. N.; Adelstein, S. J.; Burakoff, S. J. Radioimmunotherapy with alpha-particle-emitting immunoconjugates. Science 1988, 240, 1024-1026.
Mayer, E. L.; Burstein, H. J. Chemotherapy for Triple-Negative Breast Cancer: Is More Better? Journal of Clinical Oncology 2016, 34, (28), 3369-3371.
McDevitt, M. R.; Sgouros, G.; Sofou, S. Targeted and Nontargeted α-Particle Therapies. Annual Review of Biomedical Engineering 2018, 20, (1), 73-93.
Minchinton, A. I.; Tannock, I. F. Drug penetration in solid tumours. Nat Rev Cancer 2006, 6, (8), 583-592.
Oakman, C.; Viale, G.; Di Leo, A. Management of triple negative breast cancer. The Breast 2010, 19, (5), 312-321.
Ott, H. C.; Matthiesen, T. S.; Goh, S.-K.; Black, L. D.; Kren, S. M.; Netoff, T. I.; Taylor, D. A. Perfusion-decellularized matrix: using natures platform to engineer a bioartificial heart. Nature Medicine 2008, 14, 213.
Ovcaricek, T.; Frkovic, S.; Matos, E.; Mozina, B.; Borstnar, S. Triple negative breast cancer—prognostic factors and survival. Radiol Oncol 2011, 45, 46-52.
Pacheco-Torres, J.; Mukherjee, N.; Walko, M.; Lopez-Larrubia, P.; Ballesteros, P.; Cerdan, S.; Kocer, A. Image guided drug release from pH-sensitive Ion channel-functionalized stealth liposomes into an in vivo glioblastoma model. Nanomedicine: Nanotechnology, Biology and Medicine 2015, 11, 1345-1354.
Poty, S.; Francesconi, L. C.; McDevitt, M. R.; Morris, M. J.; Lewis, J. S. α-Emitters for Radiotherapy: From Basic Radiochemistry to Clinical Studies—Part 2. Journal of Nuclear Medicine 2018, 59, (7), 1020-1027.
Prabhakar, U.; Blakey, D. C.; Maeda, H.; Jain, R. K.; Sevick-Muraca, E. M.; Zamboni, W.; Farokhzad, O. C.; Barry, S. T.; Gabizon, A.; Grodzinski, P. Challenges and key considerations of the enhanced permeability and retention effect (EPR) for nanomedicine drug delivery in oncology. Cancer Research 2013, 73, 2412-2417.
Raavé, R.; van Kuppevelt, T. H.; Daamen, W. F. Chemotherapeutic drug delivery by tumoral extracellular matrix targeting. Journal of Controlled Release 2018, 274, 1-8.
Rosenbaum, P. R. Conditional permutation tests and the propensity score in observational studies. J Am Stat Assoc 1984, 79, 79:565Y74.
Sathekge, M.; Bruchertseifer, F.; Knoesen, O.; Reyneke, F.; Lawal, I.; Lengana, T.; Davis, C.; Mahapane, J.; Corbett, C.; Vorster, M.; Morgenstern, A. (225)Ac-PSMA-617 in chemotherapy-naive patients with advanced prostate cancer: a pilot study. European journal of nuclear medicine and molecular imaging 2019, 46, (1), 129-138.
Sempkowski, M.; Locke, T.; Stras, S.; Zhu, C.; Sofou, S. Liposome-Based Approaches for Delivery of Mainstream Chemotherapeutics: Preparation Methods, Liposome Designs, Therapeutic Efficacy. Crit Rev Oncology 2014, 19, (3-4), 177-221.
Sempkowski, M.; Zhu, C.; Menzenski, M.; Kevrekidis, Y.; Bruchertseifer, F.; Morgenstern, A.; Sofou, S. Triggered ligand clustering on lipid nanoparticles enables selective targeting and killing of untargetable cancer cells: the case for ‘sticky patches’. Langmuir 2016, 32, 8329-8338.
Sgouros, G. Alpha-particles for targeted therapy. Advanced Drug Delivery Reviews 2008, 60, (12), 1402-1406.
Shi, J.; Kantoff, P. W.; Wooster, R.; Farokhzad, 0. C. Cancer nanomedicine: progress, challenges and opportunities. Nat Rev Cancer 2017, 17, (1), 20-37.
Sofou, S. Radionuclide carriers for targeting of cancer. Int J Nanomed 2008, 3, 181-199.
Sofou, S.; Thomas, J. L.; Lin, H.-Y.; McDevitt, M. R.; Scheinberg, D. A.; Sgouros, G. Engineered liposomes for potential α-particle therapy of metastatic cancer. J Nucl Med 2004, 45, 253-260.
Sokolova, V.; Kozlova, D.; Knuschke, T.; Buer, J.; Westendorf, A. M.; Epple, M. Mechanism of the uptake of cationic and anionic calcium phosphate nanoparticles by cells. Acta Biomaterialia 2013, 9, (7), 7527-7535.
Song, H.; Hedayati, M.; Hobbs, R. F.; Shao, C.; Bruchertseifer, F.; Morgenstern, A.; DeWeese, T. L.; Sgouros, G. Targeting Aberrant DNA Double-Strand Break Repair in Triple-Negative Breast Cancer with Alpha-Particle Emitter Radiolabeled Anti-EGFR Antibody. Molecular Cancer Therapeutics 2013, 12, (10), 2043-2054.
Stefansson, O. A.; Villanueva, A.; Vidal, A.; Marti, L.; Esteller, M. BRCA1 epigenetic inactivation predicts sensitivity to platinum-based chemotherapy in breast and ovarian cancer. Epigenetics 2012, 7, (11), 1225-1229.
Stewart, J. C. Colorimetric determination of phospholipids with ammonium ferrothiocyanate. Anal. Biochem. 1980, 104, 10-14.
Stras, S.; Holeran, T.; Howe, A.; Sofou, S. Interstitial Release of Cisplatin from Triggerable Liposomes Enhances Efficacy Against Triple Negative Breast Cancer Solid Tumor Analogues. Molecular Pharmaceutics 2016, 13, 3224-3233.
Stras, S.; Howe, A.; Prasad, A.; Salerno, D.; Bhatavdekar, O.; Sofou, S. Growth of Metastatic Triple-Negative Breast Cancer Is Inhibited by Deep Tumor-Penetrating and Slow Tumor-Clearing Chemotherapy: The Case of Tumor-Adhering Liposomes with Interstitial Drug Release. Molecular Pharmaceutics 2019.
Stylianopoulos, T.; Poh, M.-Z.; Insin, N.; Bawendi, M. G.; Fukumura, D.; Munn, Lance L.; Jain, R. K. Diffusion of Particles in the Extracellular Matrix: The Effect of Repulsive Electrostatic Interactions. Biophysical Journal 2010, 99, (5), 1342-1349.
Telli, M. Optimizing Chemotherapy in Triple-Negative Breast Cancer: The Role of Platinum ASCO Educational Book 2014, e37-e42.
Thurber, G. M.; Zajic, S. C.; Wittrup, K. D. Theoretic Criteria for Antibody Penetration into Solid Tumors and Micrometastases. J Nucl Med 2007, 48, (6), 995-999.
van Hennik, M. B.; van der Vijgh, W. J.; Klein, I.; Elferink, F.; Vermorken, J. B.; Winograd, B.; Pinedo, H. M. Comparative pharmacokinetics of cisplatin and three analogues in mice and humans. Cancer research 1987, 47, (23), 6297-6301.
Vaupel, P. Tumor microenvironmental physiology and its implications for radiation oncology. Semin Radiat Oncol 2004, 14, 198-206.
Vaupel, P.; Kallinowski, F.; Okunieff, P. Blood Flow, Oxygen and Nutrient Supply, and Metabolic Microenvironment of Human Tumors: A Review. Cancer Res 1989, 49, (23), 6449-6465.
Wang, A. Z. EPR or no EPR? The billion-dollar question. Science Translational Medicine 2015, 7, (294), 294ec112-294ec112.
Zhu, C.; Sempkowski, M.; Holleran, T.; Linz, T.; Bertalan, T.; Josefsson, A.; Bruchertseifer, F.; Morgenstern, A.; Sofou, S. Alpha-particle radiotherapy: For large solid tumors diffusion trumps targeting. Biomaterials 2017, 130, 67-75.
Zhu, X.; Palmer, M. R.; Makrigiorgos, G. M.; Kassis, A. I. Solid-tumor radionuclide therapy dosimetry: New paradigms in view of tumor microenvironment and angiogenesis. Medical Physics 2010, 37, (6), 2974-2984.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A lipid-based nanocarrier of formula (I):
L-P—R₁ (I);
wherein:
L is a phospholipid;
P is a polyethylene glycol linker; and
R₁ is a moiety having a tritratable cationic charge that becomes positively charged under a physiological pH of a tumor interstitium;
wherein: R₁ is conjugated to a free end of the polyethylene glycol linker; the lipid-based nanocarrier adheres to a target cell or the extracellular matrix (ECM) thereof, and wherein internalization of the lipid-based nanocarrier by the target cell is minimized;
and pharmaceutically acceptable salts thereof.

2. The lipid-based nanocarrier of claim 1, wherein the compound of formula (I) has the following structure: 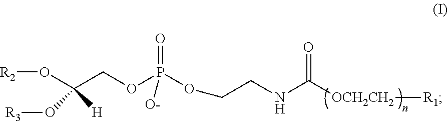
wherein:
n is an integer from 1 to 1000;
R₁ is a moiety having a tritratable cationic charge that becomes positively charged under a physiological pH of a tumor interstitium;
R₂ and R₃ are each independently a fatty acid or fatty acid residue, wherein R₂ and R₃ can be the same or different;
and pharmaceutically acceptable salts thereof.

3. The lipid-based nanocarrier of claim 1 or claim 2, wherein R₁ comprises a moiety having an intrinsic pKa having a range from about 6.0 to about 6.9.

4. The lipid-based nanocarrier of any of claims 1-3, wherein R₁ is dimethyl ammonium propane.

5. The lipid-based nanocarrier of any of claims 1-4, wherein the polyethylene glycol linker is selected from the group consisting of PEG(100), PEG(200), PEG(300), PEG(400), PEG(600), PEG(800), PEG(1000), PEG(1500), PEG(2000), PEG(3000), PEG(3350), PEG(4000), PEG(6000), PEG(8000), PEG(10,000), and PEG(35,000).

6. The lipid-based nanocarrier of claim 5, wherein the polyethylene glycol linker comprises PEG(2000).

7. The lipid-based nanocarrier of any of claims 1-6, wherein R₂ and R₃ are each independently selected from the group consisting of: butyric acid, valeric acid, caproic acid, enanthic acid, caprylic acid, pelargonic acid, capric acid, undecylic acid, lauric acid, tridecylic acid, myristic acid, pentadecylic acid, palmitic acid, margaric acid, stearic acid, nonadecylic acid, arachidic acid, heneicosylic acid, behenic acid, tricosylic acid, lignoceric acid, pentacosylic acid, cerotic acid, heptacosylic acid, montanic acid, nonacosylic acid, melissic acid, henatriacontylic acid, lacceroic acid, psyllic acid, geddic acid, ceroplastic acid, hexatriacontylic acid, heptatriacontanoic acid, octatriacontanoic acid, nonatriacontanoic acid, tetracontanoic acid, crotonic acid, myristoleic acid, palmitoleic acid, sapienic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, dihomo-γ-linolenic acid, eicosatrienoic acid; stearidonic acid, arachidonic acid, eicosatetraenoic acid, adrenic acid, bosseopentaenoic acid, eicosapentaenoic acid, ozubondo acid, sardine acid, tetracosanolpentaenoic acid, docosahexaenoic acid, and herring acid.

8. The lipid-based nanocarrier of claim 1, wherein the compound of formula (I) has the following formula:

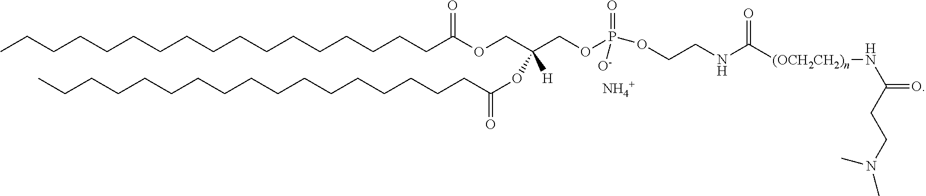

9. The lipid-based nanocarrier of any of claims 1-8, wherein the lipid-based nanocarrier further comprises one or more therapeutic agents.

10. The lipid-based nanocarrier of claim 9, wherein the one or more therapeutic agents comprises a chemotherapeutic agent or a radionuclide.

11. The lipid-based nanocarrier of claim 10, wherein the radionuclide comprises 225-Actinium.

12. The lipid-based nanocarrier of claim 10, wherein the chemotherapeutic agent comprises a platinum-based antineoplastic agent.

13. The lipid-based nanocarrier of claim 12, wherein the platinum-based antineoplastic agent is selected from the group consisting of cisplatin, carboplatin, oxaliplatin, nedaplatin, triplatin tetranitrate, phenanthriplatin, picoplatin, and satraplatin.

14. The lipid-based nanocarrier of claim 13, wherein the platinum-based antineoplastic agent comprises cisplatin.

15. A pharmaceutical formulation comprising a lipid-based nanocarrier of any of claims 1-14 and a pharmaceutically acceptable carrier.

16. A method for treating a disease, disorder, or condition, the method comprising administering a therapeutically effective amount of a lipid-based nanocarrier of any of claims 1-14, or the formulation of claim 15, to a subject in need of treatment thereof.

17. The method of claim 16, wherein the disease, disorder, or condition comprises a cancer.

18. The method of claim 17, wherein the cancer comprises a metastatic cancer.

19. The method of claim 17, wherein the cancer is selected from the group consisting of testicular cancer, ovarian cancer, cervical cancer, breast cancer, bladder cancer, head and neck cancer, esophageal cancer, lung cancer, mesothelioma, brain tumors, and neuroblastoma.

20. The method of claim 19, wherein the cancer is breast cancer.

21. The method of claim 20, wherein the breast cancer is triple negative breast cancer (TNBC).

22. The lipid-based nanocarrier of claim 10, further comprising one or more chelating agents.

23. The lipid-based nanocarrier of claim 22, wherein the one or more chelating agents is selected from the group consisting of DOTAGA (1,4,7,10-tetraazacyclododececane,1-(glutaric acid)-4,7,10-triacetic acid), DOTA (1,4,7,10-tetraazacyclododecane-1,4,7,10-tetraacetic acid), DOTASA (1,4,7,10-tetraazacyclododecane-1-(2-succinic acid)-4,7,10-triacetic acid), CB-DO2A (10-bis(carboxymethyl)-1,4,7,10-tetraazabicyclo[5.5.2]tetradecane), DEPA (7-[2-(Bis-carboxymethylamino)-ethyl]-4,10-bis-carboxymethyl-1,4,7,10-tetraaza-cyclododec-1-yl-acetic acid)), 3p-C-DEPA (2-[(carboxymethyl)][5-(4-nitrophenyl-1-[4,7,10-tris(carboxymethyl)-1,4,7,10-tetraazacyclododecan-1-yl]pentan-2-yl)amino]acetic acid)), TCMC (2-(4-isothiocyanotobenzyl)-1,4,7,10-tetraaza-1,4,7,10-tetra-(2-carbamonyl methyl)-cyclododecane), oxo-DO3A (1-oxa-4,7,10-triazacyclododecane-5-S-(4-isothiocyanatobenzyl)-4,7,10-triacetic acid), p-NH2-Bn-Oxo-DO3A (1-Oxa-4,7,10-tetraazacyclododecane-5-S-(4-aminobenzyl)-4,7,10-triacetic acid), TE2A ((1,8-N,N′-bis-(carboxymethyl)-1,4,8,11-tetraazacyclotetradecane), MM-TE2A, DM-TE2A, CB-TE2A (4,11-bis(carboxymethyl)-1,4,8,11-tetraazabicyclo[6.6.2]hexadecane), CB-TE1A1P (4,8,11-tetraazacyclotetradecane-1-(methanephosphonic acid)-8-(methanecarboxylic acid), CB-TE2P (1,4,8,11-tetraazacyclotetradecane-1,8-bis(methanephosphonic acid), TETA (1,4,8,11-tetraazacyclotetradecane-1,4,8,11-tetraacetic acid), NOTA (1,4,7-triazacyclononane-N,N′,N″-triacetic acid), NODA (1,4,7-triazacyclononane-1,4-diacetate); NODAGA (1,4,7-triazacyclononane,1-glutaric acid-4,7-acetic acid), (NOTAGA) 1,4,7-triazonane-1,4-diyl)diacetic acid DFO (Desferoxamine), NETA ([4-[2-(bis-carboxymethylamino)-ethyl]-7-carboxymethyl-[1,4,7]triazonan-1-yl}-acetic acid), TACN-TM (N,N′,N″, tris(2-mercaptoethyl)-1,4,7-triazacyclononane), Diamsar (1,8-Diamino-3,6,10,13,16,19-hexaazabicyclo(6,6,6)eicosane, 3,6,10,13,16,19-Hexaazabicyclo[6.6.6]eicosane-1,8-diamine), Sarar (1-N-(4-aminobenzyl)-3, 6,10,13,16,19-hexaazabicyclo[6.6.6] eicosane-1,8-diamine), AmBaSar (4-((8-amino-3,6,10,13,16,19-hexaazabicyclo [6.6.6] icosane-1-ylamino) methyl) benzoic acid), and BaBaSar.

24. The lipid-based nanocarrier of claim 22, wherein the one or more chelating agents is selected from the group consisting of:

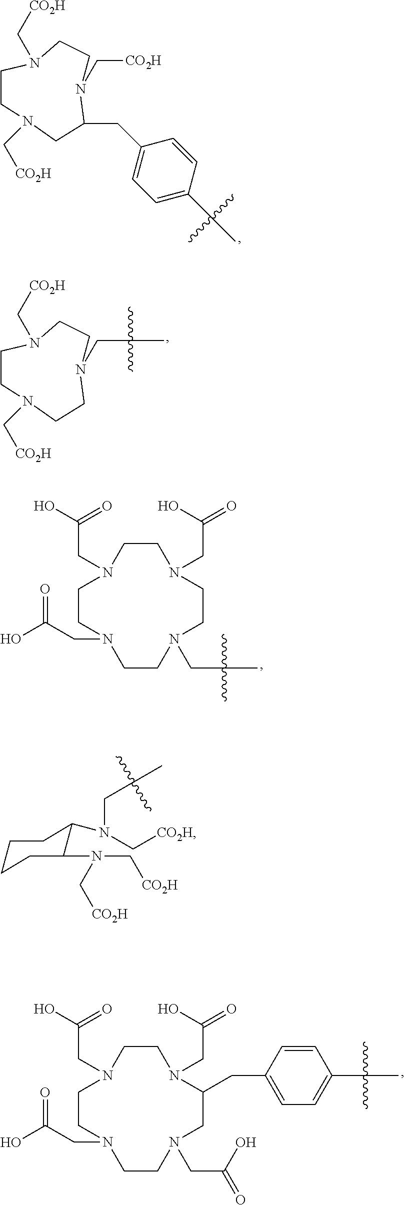 [SMILES: *c1ccc(CC2CN(CC(=O)O)CCN(CC(=O)O)CCN2CC(=O)O)cc1 ; *CN1CCN(CC(=O)O)CCN(CC(=O)O)CC1 ; *CN1CCN(CC(=O)O)CCN(CC(=O)O)CCN(CC(=O)O)CC1 ; *CN(CC(=O)O)C1CCCCC1N(CC(=O)O)CC(=O)O ; *c1ccc(CC2CN(CC(=O)O)CCN(CC(=O)O)CCN(CC(=O)O)CCN2CC(=O)O)cc1]

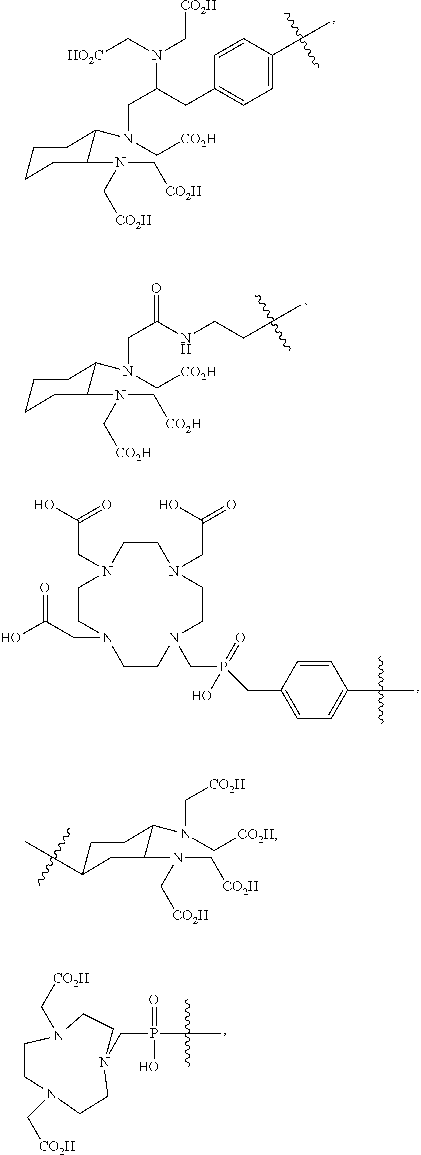 [SMILES: *c1ccc(CC(CN(CC(=O)O)C2CCCCC2N(CC(=O)O)CC(=O)O)N(CC(=O)O)CC(=O)O)cc1 ; *CCNC(=O)CN(CC(=O)O)C1CCCCC1N(CC(=O)O)CC(=O)O ; *c1ccc(CP(=O)(O)CN2CCN(CC(=O)O)CCN(CC(=O)O)CCN(CC(=O)O)CC2)cc1 ; *C1CCC(N(CC(=O)O)CC(=O)O)C(N(CC(=O)O)CC(=O)O)C1 ; *P(=O)(O)CN1CCN(CC(=O)O)CCN(CC(=O)O)CC1]

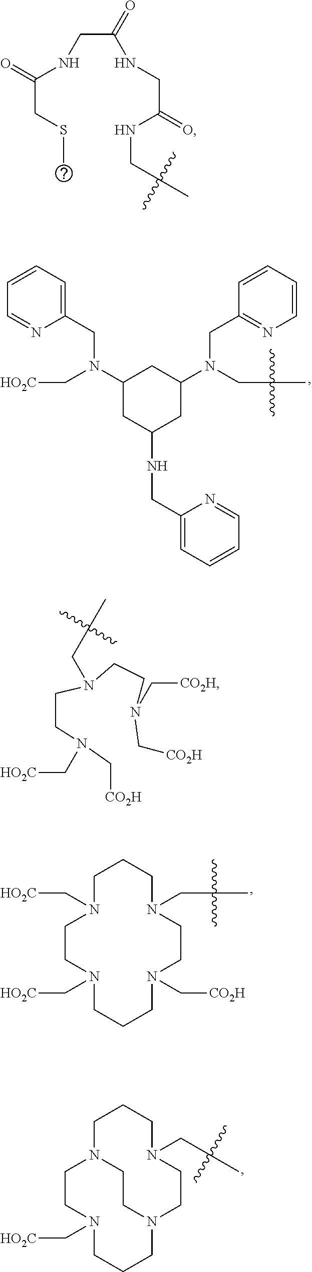 [SMILES: *CNC(=O)CNC(=O)CNC(=O)CSC ; *CN(Cc1ccccn1)C1CC(NCc2ccccn2)CC(N(CC(=O)O)Cc2ccccn2)C1 ; *CN(CCN(CC(=O)O)CC(=O)O)CCN(CC(=O)O)CC(=O)O ; *CN1CCCN(CC(=O)O)CCN(CC(=O)O)CCCN(CC(=O)O)CC1 ; *CN1CCCN2CCN(CCCN(CC(=O)O)CC2)CC1]

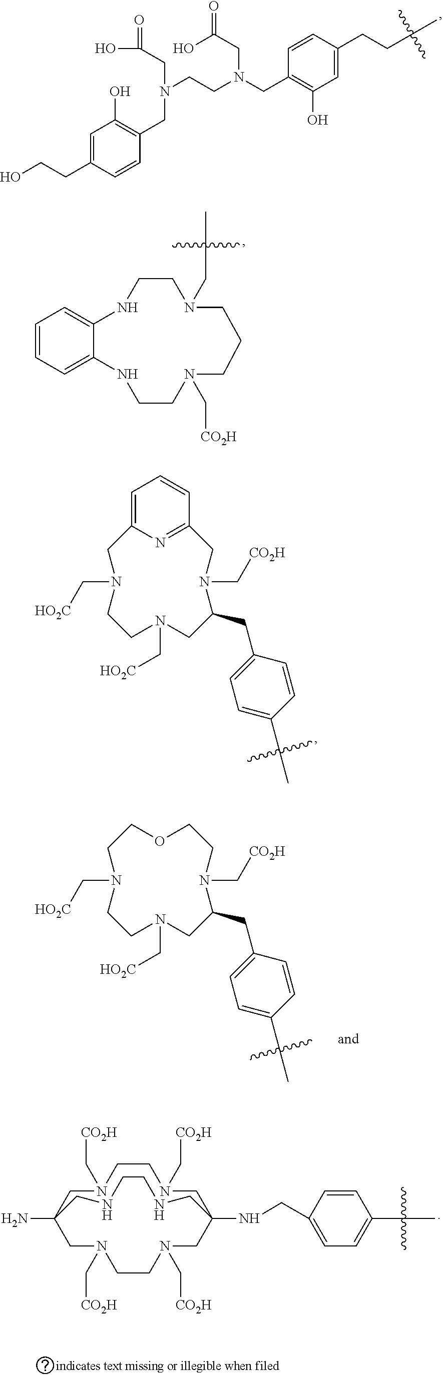 [SMILES: *CCc1ccc(CN(CCN(CC(=O)O)Cc2ccc(CCO)cc2O)CC(=O)O)c(O)c1 ; *CN1CCCN(CC(=O)O)CCNc2ccccc2NCC1 ; *c1ccc(C[C@H]2CN(CC(=O)O)CCN(CC(=O)O)Cc3cccc(n3)CN2CC(=O)O)cc1 ; *c1ccc(C[C@H]2CN(CC(=O)O)CCN(CC(=O)O)CCOCCN2CC(=O)O)cc1 ; *c1ccc(CNC23CNCCNCC(N)(CN(CC(=O)O)CCN(CC(=O)O)C2)CN(CC(=O)O)CCN(CC(=O)O)C3)cc1]

25. The lipid-based nanocarrier of claim 10, wherein the radionuclide comprises a radiometal selected from the group consisting of: ^{94m}Tc, ^{99m}Tc, ¹¹¹In, ⁶⁷Ga, ⁶⁸Ga, ⁸⁶Y, ⁹⁰Y, ¹⁷⁷Lu, ¹⁸⁶Re, ¹⁸⁸Re, ⁶⁰Cu, ⁶¹Cu, ⁶²Cu, ⁶⁴Cu, ⁶⁷Cu, ⁵⁵Co, ⁵⁷Co, ⁴⁷Sc, ²²⁵Ac, ²¹³Bi, ²¹²Bi, ²¹²Pb, ¹⁵³Sm, ¹⁶⁶Ho, ¹⁵²Gd, ⁸²Rb, ⁸⁹Zr, and ¹⁶⁶Dy.
